(19) United States
(12) Patent Application Publication
(10) Pub. No.: US 2008/0255134 A1
(43) Pub. Date: Oct. 16, 2008

(54) Cardiotonic Compounds With Inhibitory Activity Against Beta-Adrenergic Receptors And Phosphodiesterase

(75) Inventors: Malcolm George Taylor, Didcot (GB); Burkhard Klenke, Didcot (GB); Peter D. Suzdak, Towson, MD (US); Reza Mazhari, Towson, MD (US)

Correspondence Address: SEED INTELLECTUAL PROPERTY LAW GROUP PLLC, 701 FIFTH AVE, SUITE 5400, SEATTLE, WA, 98104, US

(73) Assignee: Artesian Therapeutics, Inc., Gaithersburg, MD (US)

(21) Appl. No.: 11/791,894

(22) PCT Filed: Nov. 8, 2005
(86) PCT No.: PCT/US2005/040409

§ 371 Date: Apr. 14, 2008

Related U.S. Application Data
(60) Provisional application No. 60/631,599, filed on Nov. 30, 2004.
(60) Provisional application No. 60/717,756, filed on Sep. 19, 2005.

(51) Int. Cl. A61K 31/501; C07D 401/02; C07D 401/10; A61K 31/44; C07D 401/12; A61P 9/10

(52) U.S. Cl. 514/252.03; 544/238; 544/239; 546/276.7; 546/300; 514/247; 514/339; 514/351

(57) ABSTRACT

The present invention provides compounds of formula (I) possessing inhibitory activity against β adrenergic receptors and phosphodiesterase (PDE), including type 3 phosphodiesterase (PDE-3). The present invention further provides pharmaceutical compositions comprising such compounds, methods of preparing such compounds, and methods of using such compounds for regulating calcium homeostasis, for treating a disease, disorder or condition in which disregulation of calcium homeostasis is implicated and for treating cardiovascular disease, stroke, epilepsy, an ophthalmic disorder or migraine.

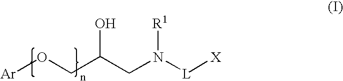

### BACKGROUND OF THE INVENTION

1. Field of the Invention

The present invention is directed to novel compounds possessing both PDE-inhibitory and β-adrenergic receptor agonist activities.

2. Description of the Related Art

Congestive heart failure affects an estimated 4.8 million Americans with over 400,000 new cases diagnosed each year. Despite incremental advances in drug therapy, the prognosis for patients with advanced heart failure remains poor with annual mortality exceeding 40 percent. Although heart transplantation is an effective therapy for patients with advanced heart failure, less than 2,200 heart transplants are performed annually due to a limited supply of donor organs. Recent analyses indicate that further increases in the incidence and prevalence of advanced heart failure are likely, highlighting the pressing need for novel and effective therapeutic strategies.

During heart failure, there is an alteration of calcium homeostasis, including impaired sarcoplasmic reticulum calcium re-uptake, increased basal (diastolic) calcium levels, decreased peak (systolic) calcium and reduced rate of calcium transients, resulting in a decreased force of contraction and a slowing of relaxation. The end results of these abnormalities in calcium homeostasis are depressed contractile function (decreased contractility and cardiac output), impaired ventricular relaxation, and myocyte loss via ischemia and/or apoptosis-related mechanisms. Disregulation of calcium homeostasis has also been implicated in a number of other disease states, including stroke, epilepsy, ophthalmic disorders, and migraine.

Beta-adrenergic blocking agents are common therapy for patients with mild to moderate chronic heart failure (CHF). Some patients on β-blockers may subsequently decompensate, however, and would need acute treatment with a positive inotropic agent. Phosphodiesterase inhibitors (PDEI), such as milrinone or enoximone, retain their full hemodynamic effects in the face of beta-blockade, because the site of PDEI action (cAMP) is downstream of the β-adrenergic receptor, and because β-antagonism reverses receptor pathway desensitization changes, which are detrimental to PDEI response.

### BRIEF SUMMARY OF THE INVENTION

The present invention provides compounds possessing inhibitory activity against β-adrenergic receptors and phosphodiesterase (PDE), including type 3 phosphodiesterase (PDE-3). The present invention further provides pharmaceutical compositions comprising such compounds, methods of preparing such compounds, and methods of using such compounds for regulating calcium homeostasis, for treating a disease, disorder or condition in which disregulation of calcium homeostasis is implicated, and for treating cardiovascular disease, stroke, epilepsy, an ophthalmic disorder or migraine.

### BRIEF DESCRIPTION OF THE SEVERAL VIEWS OF THE DRAWING(S)

FIG. 1 is a bar graph depicting the percent change in sarcomere shortening in isolated ventricular myocytes upon treatment with the indicated test compounds or isoproterenol at 0.1 μM concentration.

FIG. 2 is a graph illustrating the dose-dependent increase in LV contractility associated with the indicated test compounds.

FIG. 3 is a graph illustrating the dose-dependent percent decrease in heart rate associated with the indicated test compounds.

FIG. 4 are graphs illustrating drug administration protocol 1 and protocol 2, as used in in vivo studies on β-adrenergic blockade and PDE inhibition.

FIG. 5 is a bar graph comparing the β-adrenergic blocking effects of Compound 25 and carvedilol.

FIG. 6 is a bar graph comparing the effects of Compound 25 and carvedilol on PDE3 inhibition.

FIG. 7 is a graph plotting the baseline sarcomere length data.

FIG. 8 is a graph plotting the sarcomere length data of Compound 25.

FIG. 9 is a bar graph comparing the effects of isoproterenol, Compound 8c, atenolol, and carvedilol on contractility in ventricular myocytes.

FIG. 10 are graphs comparing the dose-dependent effects of test compounds on left ventricular contractility during isoproterenol challenge (0.5 mg/kg). FIG. 10a compares the effect of Compound 25 to carvedilol, and FIG. 10b compares the effect of Compound 8c to atenolol and carvedilol.

FIG. 11 are graphs comparing the dose-dependent effects of test compounds on heart rate during isoproterenol challenge (0.5 μg/kg). FIG. 11a compares the effect of Compound 25 to carvedilol, and FIG. 11b compares the effect of Compound 8c to atenolol and carvedilol.

FIG. 12 are graphs comparing the dose-dependent effects of test compounds on contractility (protocol 2). FIG. 12a compares the effect of Compound 25 to carvedilol, and FIG. 12b compares the effect of Compound 8c to atenolol and carvedilol.

FIG. 13 is a bar graph comparing the effects of Compound 25 and carvedilol on heart rate, left ventricular contractility, mean arterial pressure (MAP) and relaxation properties (Tau) at ED₅₀ during isoproterenol challenge.

FIG. 14 is a bar graph comparing the effects of Compound 8c, atenolol, and carvedilol on heart rate and left ventricular contractility during isoproterenol challenge.

FIG. 15 is a bar graph comparing the effects of Compound 8c, atenolol, and carvedilol on mean arterial pressure (Protocol 2; mean±SEM).

FIG. 16 is a bar graph illustrating the effect of Compound 25, with and without atropine administration and bilateral vagatomy, on heart rate (Protocol 2).

FIG. 17 is a graph comparing the dose-dependent effects of Compound 25 and carvedilol on left ventricular contractility in naive anesthetized canines (Protocol 2).

FIG. 18 is a bar graph illustrating the comparative effect of isorpoterenol, Compound 8c, carvedilol, and forskolin on cyclic-AMP production as measured by fluorescence in cardiac fibroblasts, presented as [(F−Fₒ)/Fₒ], mean±SEM, N=4.

### DETAILED DESCRIPTION OF THE INVENTION

The present invention is based upon the development of novel dual-pharmacophore small molecule compounds that possess both phosphodiesterase and β-adrenergic receptor inhibitory activity. The compounds of the present invention retain the positive attributes of β-adrenergic receptor antagonism without producing depression of cardiovascular function by simultaneously antagonizing both the β-adrenergic receptor and phosphodiesterase-3. As described herein, compounds of the present invention were found to augment cellular contractility in the absence of isoproterenol, and elicit a potent β-blocking effect antagonizing the effects of isoproterenol, in an in vivo animal model. Thus, these compounds are able to normalize β-adrenergic receptor signaling while maintaining normal myocardial contractility and, therefore, represent a new class of drugs for the treatment of heart failure and hypertension.

In certain embodiments, the compounds of the present invention comprise a phosphodiesterase inhibitor tethered to a β-adrenergic receptor inhibitor by a linker. In one embodiment, the linker is substantially cleaved in vivo, to produce degradant metabolites that are biologically active. In other embodiments, the linker is substantially stable in vivo, i.e., it is not cleaved to a substantial degree. In either embodiment, the compounds of the present invention provide advantageous pharmacokinetics over therapies that involve the concurrent treatment of a patient with separate phosphodiesterase inhibitors and β-adrenergic blockers, in part due to the ability of the dual pharmacophore to deliver both active agents to the same location, tissue, or cell, thereby ensuring that the same cells and tissues adversely affected by treatment with the β-adrenergic blocker are provided with positive inotropic support.

### DEFINITIONS

“Alkyl” refers to a saturated straight or branched chain hydrocarbon radical. Examples include without limitation methyl, ethyl, propyl, iso-propyl, butyl, iso-butyl, tert-butyl, n-pentyl and n-hexyl.

“Alkenyl” refers to an unsaturated straight or branched chain hydrocarbon radical comprising at least one carbon to carbon double bond. Examples include without limitation ethenyl, propenyl, iso-propenyl, butenyl, iso-butenyl, tert-butenyl, n-pentenyl and n-hexenyl.

“Alkynyl” refers to an unsaturated straight or branched chain hydrocarbon radical comprising at least one carbon to carbon triple bond. Examples include without limitation ethynyl, propynyl, iso-propynyl, butynyl, iso-butynyl, tert-butynyl, pentynyl and hexynyl.

“Cycloalkyl” refers to a mono- or poly-cyclic alkyl radical. Examples include without limitation cyclobutyl, cyclopentyl, cyclohexyl, cycloheptyl and cyclooctyl.

“Cycloalkenyl” refers to a mono- or poly-cyclic alkenyl radical. Examples include without limitation cyclopentenyl, cyclohexenyl, cycloheptenyl and cyclooctenyl.

“Cycloalkylene” refers to a divalent cycloalkyl radical.

“Heterocycloalkylene” refers to a divalent saturated mono- or poly-cyclic alkyl radical, in which one or more carbon atoms is/are replaced by one or more heteroatom(s), such as nitrogen, phosphorous, oxygen, sulfur, silicon, germanium, selenium and/or boron. In some embodiments, the heteroatom(s) is/are nitrogen. Nonlimiting examples of heterocycloalkylenes include piperazinyl, morpholinyl, tetrahydropyranyl, tetrahydrofuranyl, piperidinyl and pyrrolidinyl.

“Alkoxy” refers to an alkyl group bonded through an oxygen linkage.

“Alkenoxy” refers to an alkenyl group bonded through an oxygen linkage.

“Alkylthio” refers to a sulfur substituted alkyl radical.

“Aryl” refers to a cyclic aromatic hydrocarbon moiety having one or more closed ring(s). Examples include without limitation phenyl, benzyl, naphthyl, anthracenyl, phenanthracenyl and biphenyl.

“Heteroaryl” refers to a cyclic aromatic moiety having one or more closed rings with one or more heteroatom(s) (for example, sulfur, nitrogen or oxygen) in at least one ring. Examples include without limitation pyrryl, furanyl, thienyl, pyridinyl, oxazolyl, thiazolyl, benzofuranyl, benzothienyl, benzofuranyl and benzothienyl.

“Halo” refers to a fluoro, chloro, bromo or iodo radical.

“Isosteres” refer to elements, functional groups, substituents, molecules or ions having different molecular formulae but exhibiting similar or identical physical properties. For example, tetrazole is an isostere of carboxylic acid because it mimics the properties of carboxylic acid even though they have different molecular formulae. Typically, two isosteric molecules have similar or identical volumes and shapes. Ideally, isosteric molecules should be isomorphic and able to co-crystallize. Other physical properties that isosteric molecules usually share include boiling point, density, viscosity and thermal conductivity. However, certain properties may be different: dipolar moments, polarity, polarization, size and shape since the external orbitals may be hybridized differently. The term “isosteres” encompasses “bioisosteres.”

“Bioisosteres” are isosteres that, in addition to their physical similarities, share some common biological properties. Typically, bioisosteres interact with the same recognition site or produce broadly similar biological effects.

“Substituted phenyl” refers to a phenyl that is substituted with one or more substituent(s). Examples of such substituent(s) include without limitation C₁-C₆ alkyl, C₂-C₆ alkenyl, C₂-C₆ alkynyl, C₁-C₆ alkoxy, C₂-C₆ alkenyloxy, phenoxy, benzyloxy, hydroxy, carboxy, hydroperoxy, carbamido, carbamoyl, carbamyl, carbonyl, carbozoyl, amino, hydroxyamino, formamido, formyl, guanyl, cyano, cyanoamino, isocyano, isocyanato, diazo, azido, hydrazino, triazano, nitrilo, nitro, nitroso, isonitroso, nitrosamino, imino, nitrosimino, oxo, C₁-C₆ alkylthio, sulfamino, sulfamoyl, sulfeno, sulfhydryl, sulfinyl, sulfo, sulfonyl, thiocarboxy, thiocyano, isothiocyano, thioformamido, halo, haloalkyl, chlorosyl, chloryl, perchloryl, trifluoromethyl, trifluoromethoxy, iodosyl, iodyl, phosphino, phosphinyl, phospho, phosphono, arsino, selanyl, disilanyl, siloxy, silyl, silylene and carbocyclic and heterocyclic moieties.

“Effective amount” refers to the amount required to produce a desired effect, for example, regulating calcium homeostasis, treating a disease, condition in which disregulation of calcium homeostasis is implicated, treating cardiovascular disease, stroke, epilepsy, an ophthalmic disorder or migraine, or inhibiting a β-adrenergic receptor and/or PDE, including PDE-3.

“Metabolite” refers to a substance produced by metabolism or by a metabolic process.

“Pharmaceutically acceptable carrier” refers to a pharmaceutically acceptable material, composition or vehicle, such as a liquid or solid filler, diluent, excipient or solvent encapsulating material, involved in carrying or transporting the subject compound from one organ, or portion of the body, to another organ or portion of the body. Each carrier is “acceptable” in the sense of being compatible with the other ingredients of the formulation and suitable for use with the patient. Examples of materials that can serve as a pharmaceutically acceptable carrier include without limitation: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

“Pharmaceutically acceptable equivalent” includes, without limitation, pharmaceutically acceptable salts, hydrates, solvates, metabolites, prodrugs and isosteres. Many pharmaceutically acceptable equivalents are expected to have the same or similar in vitro or in vivo activity as the compounds of the invention.

“Pharmaceutically acceptable salt” refers to an acid or base salt of the inventive compounds, which salt possesses the desired pharmacological activity and is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

“Prodrug” refers to a derivative of the inventive compounds that undergoes biotransformation, such as metabolism, before exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds using conventional methods, such as that described in BURGER'S MEDICINAL CHEMISTRY AND DRUG CHEMISTRY, Fifth Ed., Vol. 1, pp. 172-178, 949-982 (1995).

“Isomers” refer to compounds having the same number and kind of atoms, and hence the same molecular weight, but differing with respect to the arrangement or configuration of the atoms.

“Stereoisomers” refer to isomers that differ only in the arrangement of the atoms in space.

“Diastereoisomers” refer to stereoisomers that are not mirror images of each other. Diastereoisomers occur in compounds having two or more asymmetric carbon atoms; thus, such compounds have 2ⁿ optical isomers, where n is the number of asymmetric carbon atoms.

“Enantiomers” refers to stereoisomers that are non-superimposable mirror images of one another.

“Enantiomer-enriched” refers to a mixture in which one enantiomer predominates.

“Racemic” refers to a mixture containing equal parts of individual enantiomers.

“Non-racemic” refers to a mixture containing unequal parts of individual enantiomers.

“Animal” refers to a living organism having sensation and the power of voluntary movement, and which requires for its existence oxygen and organic food. Examples include, without limitation, members of the human, equine, porcine, bovine, murine, canine and feline species. In the case of a human, an “animal” may also be referred to as a “patient.”

“Mammal” refers to a warm-blooded vertebrate animal.

“Calcium homeostasis” refers to the internal equilibrium of calcium in a cell.

“Cardiovascular disease” refers to a disease of the heart, blood vessels or circulation.

“Heart failure” refers to the pathophysiologic state in which an abnormality of cardiac function is responsible for the failure of the heart to pump blood at a rate commensurate with the requirements of the metabolizing tissues.

“Congestive heart failure” refers to heart failure that results in the development of congestion and edema in the metabolizing tissues.

“Hypertension” refers to elevation of systemic blood pressure.

“SA/AV node disturbance” refers to an abnormal or irregular conduction and/or rhythm associated with the sinoatrial (SA) node and/or the atrioventricular (AV) node.

“Arrhythmia” refers to abnormal heart rhythm. In arrhythmia, the heartbeats may be too slow, too fast, too irregular or too early. Examples of arrhythmia include, without limitation, bradycardia, fibrillation (atrial or ventricular) and premature contraction.

“Hypertrophic subaortic stenosis” refers to enlargement of the heart muscle due to pressure overload in the left ventricle resulting from partial blockage of the aorta.

“Angina” refers to chest pain associated with partial or complete occlusion of one or more coronary arteries in the heart.

“Treating” refers to: (i) preventing a disease, disorder or condition from occurring in an animal that may be predisposed to the disease, disorder and/or condition but has not yet been diagnosed as having it; (ii) inhibiting a disease, disorder or condition, i.e., arresting its development; and/or (iii) relieving a disease, disorder or condition, i.e., causing regression of the disease, disorder and/or condition.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

### Compounds

The present invention provides a compound of formula I:

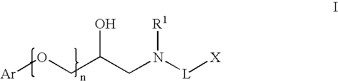

or a pharmaceutically acceptable equivalent, an isomer or a mixture of isomers thereof, wherein:

n is 0 or 1;

Ar is an aryl or heteroaryl radical, which aryl or heteroaryl radical is optionally substituted with 1 to 3 substituent(s) selected from R², R³ and R⁴;

R¹ is hydrogen, C₁-C₈ alkyl, C₂-C₈ alkenyl, C₂-C₈ alkynyl, C₃-C₈ cycloalkyl or C₃-C₈ cycloalkenyl;

R², R³ and R⁴ are independently cyano, nitro, halo, trifluoromethyl, trifluoromethoxy, acylaminoalkyl, NHR⁵, —NHSO₂R¹, —NHCONHR¹, C₁-C₄ alkoxy, C₁-C₄ alkylthio, C₁-C₈ alkyl, C₂-C₈ alkenyl or C₂-C₈ alkynyl, wherein one or more —CH₂— group(s) of the alkyl, alkenyl or alkynyl is/are optionally replaced with —O—, —S—, —SO₂— and/or —NR⁵—, and the alkyl, alkenyl or alkynyl is optionally substituted with one or more oxo(s), carbonyl oxygen(s) and/or hydroxyl(s);

L is C₁-C₁₂ alkylene, C₂-C₁₂ alkenylene or C₂-C₁₂ alkynylene, wherein one or more —CH₂— group(s) of the alkylene, alkenylene or alkynylene is/are optionally replaced with —O—, —S—, —SO₂—, —NR⁵—, C₃-C₈ cycloalkylene and/or C₃-C₈ heterocycloalkylene, and the alkylene, alkenylene and alkynylene are unsubstituted or substituted with one or more oxo(s), carbonyl oxygen(s) and/or hydroxyl(s);

R⁵ is hydrogen, a lone pair of electrons, C₁-C₈ alkyl, C₂-C₈ alkenyl or C₃-C₈ alkynyl, which alkyl, alkenyl or alkynyl is optionally substituted with phenyl or substituted phenyl;

X is a moiety of formula A, B, C, D, E, F, G, H, I, J, K, L, M, N, O, P, Q, R, S, T, U, V, W, or Y,

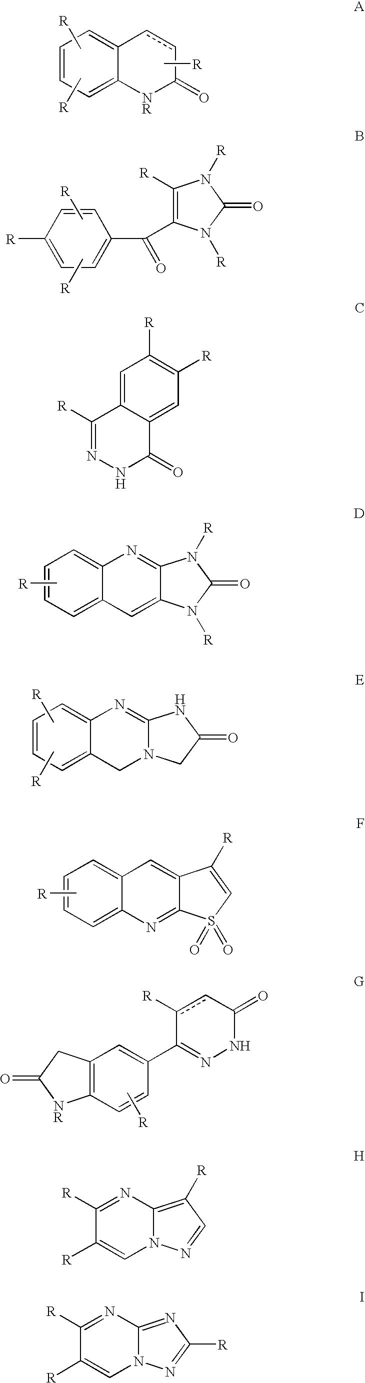

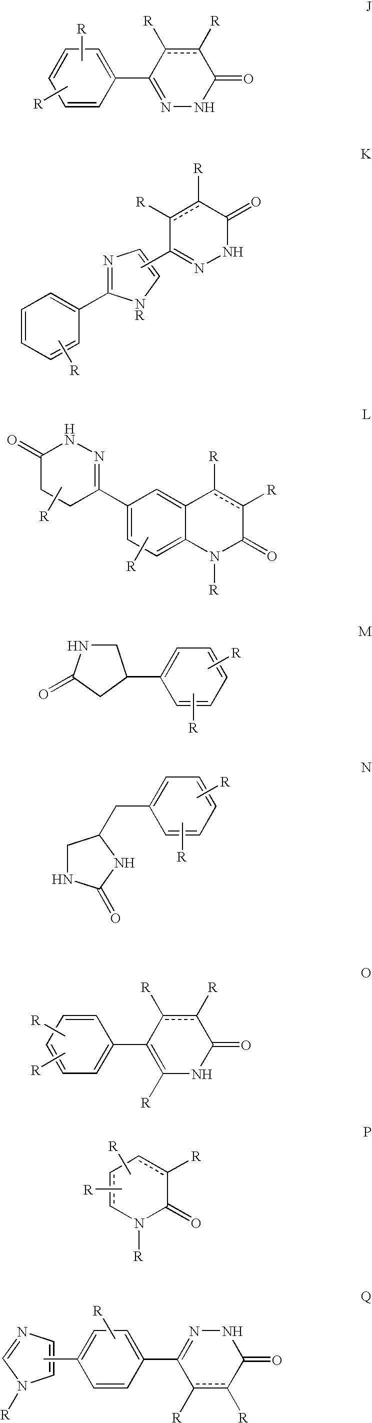

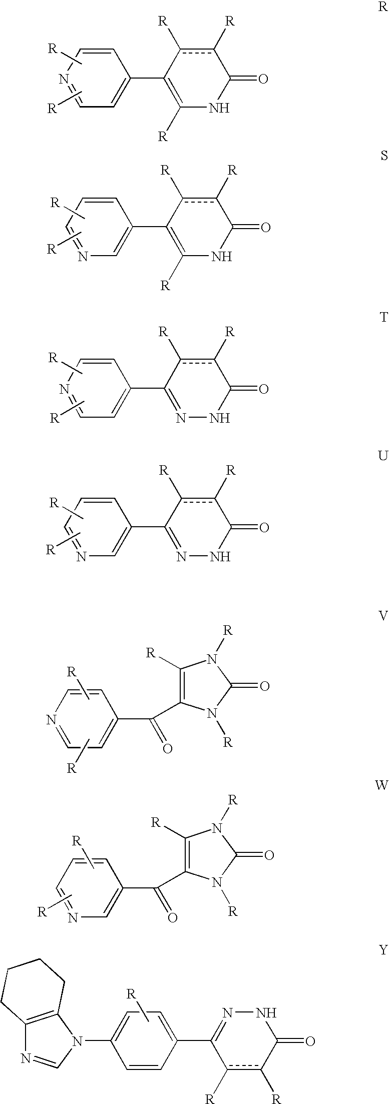

with X bonded to L through any one R; and

each R is independently a direct bond, hydrogen, halo, nitro, cyano, trifluoromethyl, trifluoromethoxy, amino, NR⁵R⁶, C₁-C₄ alkoxy, C₁-C₄ alkylthio, COOR⁷, C₁-C₁₂ alkyl, C₂-C₁₂ alkenyl or C₂-C₁₂ alkynyl, wherein one or more —CH₂— group(s) of the alkyl, alkenyl or alkynyl is/are optionally replaced with —O—, —S—, —SO₂— and/or —NR¹, and the alkyl, alkenyl or alkynyl is optionally substituted with one or more oxo(s), carbonyl oxygen(s) and/or hydroxyl(s).

Every variable substituent is defined independently at each occurrence. Thus, the definition of a variable substituent in one part of a formula is independent of its definition(s) elsewhere in that formula and of its definition(s) in other formulas.

In formula I, moieties A, G, J-L, O-U and Y contain dashed lines in their respective structures. These dashed lines indicate that saturation is optional.

In some embodiments, formula I's Ar is phenyl, benzyl, naphthyl or biphenyl. In other embodiments, Ar is phenyl which is unsubstituted or substituted with 1 to 3 substituent(s) selected from R², R³ and R⁴, wherein R², R³ and R⁴ are independently cyano, halo, trifluoromethyl, trifluoromethoxy, C₁-C₄ alkoxy, C₁-C₈ alkyl or C₂-C₈ alkenyl, wherein one or more —CH₂— group(s) of the alkyl or alkenyl is/are optionally replaced with —O—, and the alkyl or alkenyl is optionally substituted with oxo.

In some embodiments, formula (I)'s Ar is chosen from groups Ar₁, Ar₂, Ar₃, Ar₄, Ar₅, Ar₆ and Ar₇:

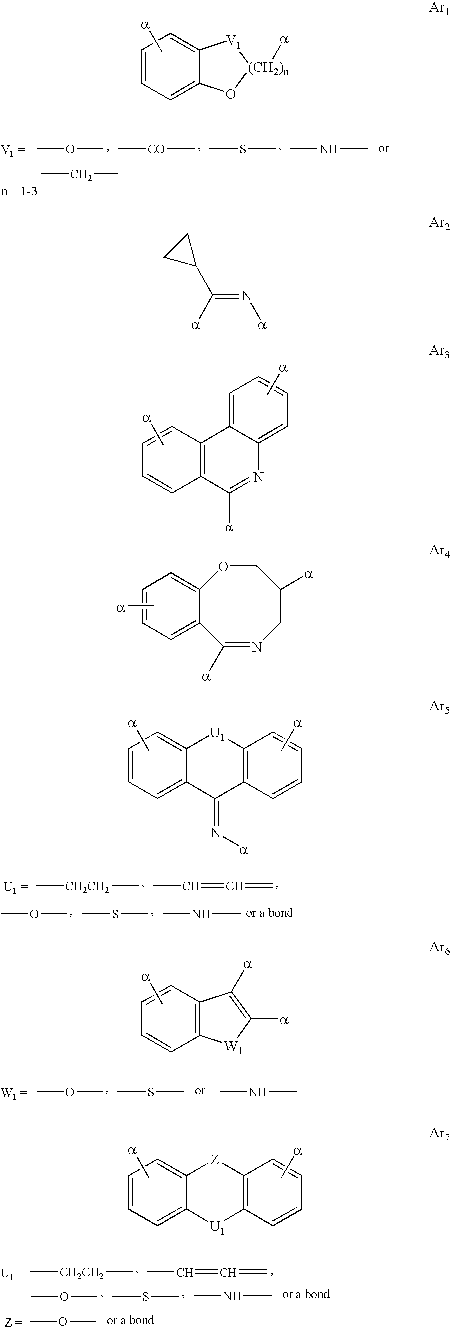

wherein α indicates the position position where Ar may bond. In further embodiments, Ar is phenyl or Ar₇, wherein Z is a bond. In yet further embodiments, U₁ in Ar₇ is NH.

In some embodiments, when formula I's X is a moiety of formula A, B, C, D, E, F, G, H, I, J, K, L, M, N, O, P, Q, R, S, T, U, V, W, or Y, then either (1) Ar is group Ar₇, wherein Z is a bond, or (2) L is C₁-C₁₂ alkylene, C₂-C₁₂ alkenylene or C₂-C₁₂ alkynylene, wherein one or more —CH₂— group(s) of the alkylene, alkenylene and alkynylene is/are replaced with C₃-C₈ cycloalkylene and/or C₃-C₈ heterocycloalkylene. In other embodiments, formula I's X is a moiety of formula J. In yet other embodiments, each R in the moiety of formula J is independently a direct bond, hydrogen or halo. In even other embodiments, X is

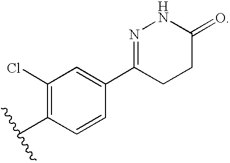

In some embodiments, formula I's L is C₁-C₁₂ alkylene, C₂-C₁₂ alkenylene or C₂-C₁₂ alkynylene, wherein one or more —CH₂— group(s) of the alkylene, alkenylene or alkynylene is/are replaced with C₃-C₈ cycloalkylene and/or C₃-C₈ heterocycloalkylene. In other embodiments, the C₃-C₈ heterocycloalkylene is piperidinylene. In yet other embodiments, the piperidinylene is piperidin-1,4-ylene or piperidin-1,3-ylene. In further embodiments, one or more —CH₂— group(s) of the alkylene, alkenylene or alkynylene is/are further replaced with —O—, and the alkylene, alkenylene or alkynylene is substituted with one or more oxo(s). In even further embodiments, L is

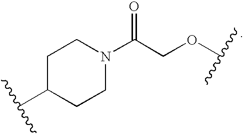

In some embodiments, formula I's R¹ is hydrogen or C₁-C₈ alkyl. In other embodiments, R¹ is hydrogen or C₁-C₄ alkyl. In yet other embodiments, R¹ is hydrogen.

In some embodiments, formula I's n is 1.

In some embodiments, the compound of the present invention is chosen from pharmaceutically acceptable salts of compounds of formula I.

In some embodiments, the compound of the present invention is chosen from hydrates of compounds of formula I.

In some embodiments, the compound of the present invention is chosen from solvates of compounds of formula I.

In some embodiments, the compound of the present invention is chosen from metabolites of compounds of formula I.

In some embodiments, the compound of the present invention is chosen from prodrugs of compounds of formula I.

In some embodiments, the compound of the present invention is chosen from isosteres of compounds of formula I.

In some embodiments, the compound of the present invention is chosen from those of formula I as defined above, pharmaceutically acceptable equivalents and isomers or mixtures of isomers thereof, wherein:

n is 1;

Ar is group Ar⁷, wherein Z is a bond and U is —NH—;

R¹ is hydrogen;

L is as defined above; and

X is as defined above. In further embodiments, X is a moiety of formula J. In yet further embodiments, each R in the moiety of formula J is independently a direct bond, hydrogen or halo. In yet further embodiments, X is

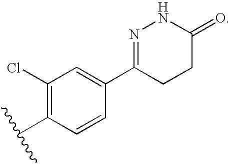

In yet further embodiments, L is C₁-C₁₂ alkylene, C₂-C₁₂ alkenylene or C₂-C₁₂ alkynylene, wherein one or more —CH₂— group(s) of the alkylene, alkenylene or alkynylene is/are optionally replaced with —O—, —S—, —SO₂— and/or —NR⁵—, and the alkylene, alkenylene and alkynylene are unsubstituted or substituted with one or more oxo(s), carbonyl oxygen(s) and/or hydroxyl(s). In yet further embodiments, L is C₁-C₈ alkylene wherein one or more —CH₂— group(s) of the alkylene is/are replaced with —O—. In yet further embodiments, L is —(CH₂)₂O—, —(CH₂)₃O— or —(CH₂)₄O—. In even further embodiments, the compound of the present invention is a racemic mixture.

In some embodiments, the compound of the present invention is chosen from those of formula I as defined above, pharmaceutically acceptable equivalents and isomers or mixtures of isomers thereof, wherein:

n is 1;

Ar is as defined above;

R¹ is hydrogen;

L is C₁-C₁₂ alkylene, C₂-C₁₂ alkenylene or C₂-C₁₂ alkynylene, wherein one or more —CH₂— group(s) of the alkylene, alkenylene or alkynylene is/are replaced with C₃-C₈ cycloalkylene and/or C₃-C₈ heterocycloalkylene; and

X is as defined above. In further embodiments, X is a moiety of formula J. In yet further embodiments, each R in the moiety of formula J is independently a direct bond hydrogen or halo. In yet further embodiments, X is

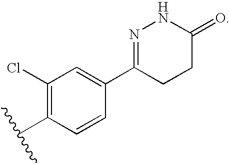

In yet further embodiments, L is C₁-C₁₂ alkylene, wherein one or more —CH₂— group(s) of the alkylene is/are replaced with C₃-C₈ cycloalkylene and/or C₃-C₈ heterocycloalkylene. In yet other embodiments, the C₃-C₈ heterocycloalkylene is piperidinylene. In yet further embodiments, the piperidinylene is piperidin-1,4-ylene or piperidin-1,3-ylene. In yet further embodiments, one or more —CH₂— group(s) of the alkylene, alkenylene or alkynylene is/are further replaced with —O—, and the alkylene, alkenylene or alkynylene is substituted with one or more oxo(s). In yet further embodiments, L is

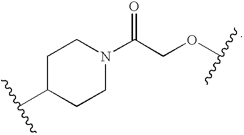

In yet further embodiments, Ar is phenyl which is unsubstituted or substituted with 1 to 3 substituent(s) selected from R², R³ and R⁴. In yet further embodiments, Ar is phenyl which is unsubstituted or substituted with 1 substitution selected from R². In yet further embodiments, R² is cyano, halo, trifluoromethyl, trifluoromethoxy, C₁-C₄ alkoxy, C₁-C₈ alkyl or C₂-C₈ alkenyl, wherein one or more —CH₂— group(s) of the alkyl or alkenyl is/are optionally replaced with —O—, and the alkyl or alkenyl is optionally substituted with oxo. In even further embodiments, the compound of the present invention is a non-racemic mixture.

Nonlimiting examples of compounds of the present invention include:

6-(3-chloro-4-{2-[4-(2-hydroxy-3-phenoxy-propylamino)-piperidin-1-yl]-2-oxo-ethoxy}-phenyl)-4,5-dihydro-2H-pyridazin-3-one (8a);

6-[3-Chloro-4-(2-{4-[3-(2-fluoro-phenoxy)-2-hydroxy-propylamino]-piperidin-1-yl}-2-oxo-ethoxy)-phenyl]-4,5-dihydro-2H-pyridazin-3-one(8b);

6-[3-Chloro-4-(2-{4-[3-(2-chloro-phenoxy)-2-hydroxy-propylamino]-piperidin-1-yl}-2-oxo-ethoxy)-phenyl]-4,5-dihydro-2H-pyridazin-3-one (8c);

6-[4-(2-{4-[3-(2-bromo-phenoxy)-2-hydroxy-propylamino]-piperidin-1-yl}-2-oxo-ethoxy)-3-chloro-phenyl]-4,5-dihydro-2H-pyridazin-3-one (8d);

2-[3-(1-{2-[2-chloro-4-(6-oxo-1,4,5,6-tetrahydro-pyridazin-3-yl)-phenoxy]-acetyl}-piperidin-4-ylamino)-2-hydroxy-propoxy]-benzonitrile (8e);

6-(3-chloro-4-{2-[4-(2-hydroxy-3-o-tolyloxy-propylamino)-piperidin-1-yl]-2-oxo-ethoxy}-phenyl)-4,5-dihydro-2H-pyridazin-3-one (8f);

6-[3-chloro-4-(2-{4-[2-hydroxy-3-(2-trifluoromethyl-phenoxy)-propylamino]-piperidin-1-yl}-2-oxo-ethoxy)-phenyl]-4,5-dihydro-2H-pyridazin-3-one (8g);

6-[3-chloro-4-(2-{4-[2-hydroxy-3-(2-methoxy-phenoxy)-propylamino]-piperidin-1-yl}-2-oxo-ethoxy)-phenyl]-4,5-dihydro-2H-pyridazin-3-one (8h);

6-[3-chloro-4-(2-{4-[2-hydroxy-3-(2-trifluoromethoxy-phenoxy)-propylamino]-piperidin-1-yl}-2-oxo-ethoxy)-phenyl]-4,5-dihydro-2H-pyridazin-3-one (8i);

6-[4-(2-{4-[3-(2-allyl-phenoxy)-2-hydroxy-propylamino]-piperidin-1-yl}-2-oxo-ethoxy)-3-chloro-phenyl]-4,5-dihydro-2H-pyridazin-3-one (8j);

6-[4-(2-{4-[3-(2-acetyl-phenoxy)-2-hydroxy-propylamino]-piperidin-1-yl}-2-oxo-ethoxy)-3-chloro-phenyl]-4,5-dihydro-2H-pyridazin-3-one (8k);

6-[4-(2-{4-[3-(3-bromo-phenoxy)-2-hydroxy-propylamino]-piperidin-1-yl}-2-oxo-ethoxy)-3-chloro-phenyl]-4,5-dihydro-2H-pyridazin-3-one (8l);

3-[3-(1-{2-[2-chloro-4-(6-oxo-1,4,5,6-tetrahydro-pyridazin-3-yl)-phenoxy]-acetyl}-piperidin-4-ylamino)-2-hydroxy-propoxy]-benzonitrile (8m);

6-[4-(2-{4-[3-(4-bromo-phenoxy)-2-hydroxy-propylamino]-piperidin-1-yl}-2-oxo-ethoxy)-3-chloro-phenyl]-4,5-dihydro-2H-pyridazin-3-one (8n);

4-[3-(1-{2-[2-chloro-4-(6-oxo-1,4,5,6-tetrahydro-pyridazin-3-yl)-phenoxy]-acetyl}-piperidin-4-ylamino)-2-hydroxy-propoxy]-benzonitrile (8o);

6-(3-chloro-4-{2-[4-(2-hydroxy-3-p-tolyloxy-propylamino)-piperidin-1-yl]-2-oxo-ethoxy}-phenyl)-4,5-dihydro-2H-pyridazin-3-one (8p);

6-[3-chloro-4-(2-{4-[2-hydroxy-3-(4-methoxy-phenoxy)-propylamino]-piperidin-1-yl}-2-oxo-ethoxy)-phenyl]-4,5-dihydro-2H-pyridazin-3-one (8q);

6-{3-chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (8r);

6-(4-{3-[3-(9H-carbazol-4-yloxy)-2-hydroxy-propylamino]-propoxy}-3-chloro-phenyl)-4,5-dihydro-2H-pyridazin-3-one (29);

6-(4-{2-[3-(9H-carbazol-4-yloxy)-2-hydroxy-propylamino]-ethoxy}-3-chloro-phenyl)-4,5-dihydro-2H-pyridazin-3-one (22);

6-(4-{2-[3-(9H-carbazol-4-yloxy)-2-hydroxy-propylamino]-ethoxy}-3-chloro-phenyl)-4,5-dihydro-2H-pyridazin-3-one (25);

6-[4-(2-{4-[3-(9H-carbazol-4-yloxy)-2-hydroxy-propylamino]-piperidin-1-yl}-2-oxo-ethoxy)-3-chloro-phenyl]-4,5-dihydro-2H-pyridazin-3-one (17, 68);

6-{3-chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (13);

2′-(2-{4-[3-(2-cyano-phenoxy)-2-hydroxy-propylamino]-piperidin-1-yl}-ethoxy)-2-methyl-6-oxo-1,6-dihydro-[3,4′]bipyridinyl-5-carbonitrile (137);

6-[4-(2-{4-[(S)-3-(9H-Carbazol-2-yloxy)-2-hydroxy-propylamino]-piperidin-1-yl}-2-oxo-ethoxy)-3-chloro-phenyl]-4,5-dihydro-2H-pyridazin-3-one (33);

6-(4-{2-[3-(9H-carbazol-2-yloxy)-2-hydroxy-propylamino]-ethoxy}-3-chloro-phenyl)-4,5-dihydro-2H-pyridazin-3-one (34);

6-(4-{3-[(S)-3-(9H-Carbazol-2-yloxy)-2-hydroxy-propylamino]-propoxy}-3-chloro-phenyl)-4,5-dihydro-2H-pyridazin-3-one (35);

6-[3-Chloro-4-(3-{(S)-2-hydroxy-propylamino]-propoxy}-phenyl)-4,5-dihydro-2H-pyridazin-3-one (36);

2-[2-Chloro-4-(6-oxo-1,4,5,6-tetrahydro-pyridazin-3-yl)-phenoxy]-N-(2-{(S)-2-hydroxy-3-[4-(2-methoxy-ethyl)-phenoxy]-propylamino}-2-methyl-propyl)-acetamide (40);

6-[3-Chloro-4-(3-{(S)-2-hydroxy-propylamino]-ethoxy}-phenyl)-4,5-dihydro-2H-pyridazin-3-one (41);

6-{3-Chloro-4-[2-(3-{(S)-2-hydroxy-3-[4-(2-methoxy-ethyl)-phenoxy]-propylamino}-pyrrolidin-1-yl)-2-oxo-ethoxy]-phenyl}-4,5-dihydro-2H-pyridazin-3-one (42);

6-[3-Chloro-4-(3-{(S)-2-hydroxy-propylamino]-butoxy}-phenyl)-4,5-dihydro-2H-pyridazin-3-one (44);

6-[3-Chloro-4-(3-{(S)-2-hydroxy-propylamino]-pentoxy}-phenyl)-4,5-dihydro-2H-pyridazin-3-one (46);

N-{2-[3-(9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (49);

6-{3-Chloro-4-[2-(3-{(S)-2-hydroxy-3-[4-(2-methoxy-ethyl)-phenoxy]-propylamino}-azetidin-1-yl)-2-oxo-ethoxy]-phenyl}-4,5-dihydro-2H-pyridazin-3-one (48);

6-(3-Chloro-4-{2-hydroxy-3-(1H-indol-4-yloxy)-propylamino]-ethoxy}-phenyl)-4,5-dihydro-2H-pyridazin-3-one (52);

6-(3-Chloro-4-{2-hydroxy-3-(3-propylamino-phenoxy)-propylamino]-ethoxy}-phenyl)-4,5-dihydro-2H-pyridazin-3-one (55);

6-(4-{3-[3-(9H-Carbazol-4-yloxy)-2-hydroxy-butylamino]-propoxy}-3-chloro-phenyl)-4,5-dihydro-2H-pyridazin-3-one (56);

6-(4-{3-[3-(9H-Carbazol-4-yloxy)-2-hydroxy-pentylamino]-propoxy}-3-chloro-phenyl)-4,5-dihydro-2H-pyridazin-3-one (57);

6-(4-{3-[3-(9H-Carbazol-4-yloxy)-2-hydroxy-pentylamino]-propoxy}-3-chloro-phenyl)-4,5-dihydro-2H-pyridazin-3-one (61);

6-[6-(2-{4-[3-(9H-Carbazol-4-yloxy)-2-hydroxy-propylamino]-piperidin-1-yl]-4,5-dihydro-2H-pyridazin-3-one (62);

6-[4-(2-{4-[3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (63);

6-(4-{3-[3-(9H-Carbazol-4-yloxy)-2-hydroxy-propylamino]-propoxy}-3-chloro-phenyl)-4,5-dihydro-2H-pyridazin-3-one (64);

6-(4-{3-[3-(9H-Carbazol-4-yloxy)-2-hydroxy-propylamino]-propoxy}-3-chloro-phenyl)-4,5-dihydro-2H-pyridazin-3-one (69);

6-(4-{3-[3-(9H-Carbazol-4-yloxy)-2-hydroxy-propylamino]-propoxy}-3-chloro-phenyl)-4,5-dihydro-2H-pyridazin-3-one (70);

N-{2-[3-(3-Bromo-9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (72a);

N-{2-[3-(1-Bromo-9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (72b);

2-(2-Chloro-4-(6-oxo-1,4,5,6-tetrahydro-pyridazin-3-yl)-phenoxy]-N-{2-[2-hydroxy-3-(9-methyl-9H-carbazol-yloxy)-propylamino]-2-methyl-propyl}-2-methyl-propyl}-acetamide (74);

6-[4-(2-{3-[3-(9H-Carbazol-4-yloxy)-2-hydroxy-propylamino]-azetidin-1-yl}-2-oxo-ethoxy)-3-chloro-phenyl]-4,5-dihydro-2H-pyridazin-3-one (75);

9-(2-methoxy-ethyl)-4-oxiranylmethoxy-9H-carbazole (80);

2-[2-chloro-4-(6-oxo-1,4,5,6-tetrahydro-pyridazin-3-yl)-phenoxy]-N-(2-{2-hydroxy-3-[9-(2-methoxy-ethyl)-9H-carbazol-4-yloxy]-propylamino}-2-methyl-propyl)-acetamide (81);

Benzoic acid 2-(4-oxiranylmethoxy-carbazol-9-yl)-ethyl ester (84);

2-[2-Chloro-4-(6-oxo-1,4,5,6-tetrahydro-pyridazin-3-yl)-phenoxy]-N-(2-{2-hydroxy-3-[9-(2-hydroxy-ethyl)-9H-carbazol-4-yloxy]-propylamino}-2-methyl-propyl)-acetamide (85);

5-[4-(3-amino-propoxy)-phenyl]-6-methyl-2-oxo-1,2-dihydro-pyridine-3-carbonitrile (90a);

5-(4-{3-[3-(9H-carbazol-4-yloxy)-2-hydroxy-propylamino]-propoxy}-phenyl)-6-methyl-2-oxo-1,2-dihydro-pyridine-3-carbonitrile (91);

5-[4-(4-amino-butoxy)-3-chloro-phenyl]-6-methyl-2-oxo-1,2-dihydro-pyridine-3-carbonitrile (90b);

5-[4-(5-amino-pentyloxy)-3-chloro-phenyl]-6-methyl-2-oxo-1,2-dihydro-pyridine-3-carbonitrile (90c);

6-[4-(3-{(S)-2-hydroxy-propylamino]-butoxy}-phenyl)-4,5-dihydro-2H-pyridazin-3-one (92);

5-(4-{5-[3-(9H-carbazol-4-yloxy)-2-hydroxy-propylamino]-pentyloxy}-3-chloro-phenyl)-6-methyl-2-oxo-1,2-dihydro-pyridine-3-carbonitrile (93);

[4-(5-cyano-2-methyl-6-oxo-1,6-dihydro-pyridin-3-yl)-phenoxy]-acetic acid (97);

5-[4-(2-{4-[3-(9H-carbazol-4-yloxy)-2-hydroxy-propylamino]-piperidin-1-yl}-2-oxo-ethoxy)-phenyl]-6-methyl-2-oxo-1,2-dihydro-pyridine-3-carbonitrile (98);

N-{2-[3-(9H-carbazol-4-yloxy)-2-hydroxy-propylamino]-2-methyl-propyl}-2-[2-chloro-4-(5-cyano-2-methyl-6-oxo-1,6-dihydro-pyridin-3-yl)-phenoxy]-acetamide (99); and

6-[4-(2-{3-[3-(9H-Carbazol-4-yloxy)-2-hydroxy-propylamino]-pyrrolidin-1-yl}-2-oxo-ethoxy)-3-chloro-phenyl]-4,5-dihydro-2H-pyridazin-3-one (139).

Since the compounds of the present invention may possess one or more asymmetric carbon center(s), they may be capable of existing in the form of optical isomers as well as in the form of racemic or non-racemic mixtures of optical isomers. The optical isomers can be obtained by resolution of the racemic mixtures according to conventional processes. One such process entails formation of diastereoisomeric salts by treatment with an optically active acid or base, then separation of the mixture of diastereoisomers by crystallization, followed by liberation of the optically active bases from the salts. Examples of appropriate acids are tartaric, diacetyltartaric, dibenzoyltartaric, ditoluoyltartaric and camphorsulfonic acid.

A different process for separating optical isomers involves the use of a chiral chromatography column optimally chosen to maximize the separation of the enantiomers. Still another available process involves synthesis of covalent diastereoisomeric molecules, for example, esters, amides, acetals and ketals, by reacting the inventive compounds with an optically active acid in an activated form, an optically active diol or an optically active isocyanate. The synthesized diastereoisomers can be separated by conventional means such as chromatography, distillation, crystallization or sublimation, and then hydrolyzed to deliver the enantiomerically pure compound. In some cases hydrolysis to the “parent” optically active drug is not necessary prior to dosing the patient, since the compound can behave as a prodrug. The optically active compounds of the present invention likewise can be obtained by utilizing optically active starting materials.

It is understood that the compounds of the present invention encompass individual optical isomers as well as racemic and non-racemic mixtures. In some non-racemic mixtures, the R configuration may be enriched while in other non-racemic mixtures, the S configuration may be enriched.

In one embodiment, a compound of the present invention has a phosphodiesterase-3 inhibition IC₅₀ value of less than 1 μM, while in other embodiments, a compound of the present invention has a phosphodiesterase-3 inhibition IC₅₀ value of less than 500 nM or less than 100 nM.

In one embodiment, a compound of the present invention has a non-specific beta-adrenergic blockade IC₅₀ value of less than 1 μM, while in other embodiments, a compound of the present invention has a non-specific beta-adrenergic blockade IC₅₀ value of less than 500 nM or less than 100 nM.

### Methods of Use

The present invention further provides a method for regulating calcium homeostasis, comprising administering an effective amount of a compound of the present invention to an animal in need of such regulation.

The present invention further provides a method for treating a disease, disorder or condition in which disregulation of calcium homeostasis is implicated, comprising administering an effective amount of a compound of the present invention to an animal in need of such treatment.

The present invention further provides a method for treating a cardiovascular disease, stroke, epilepsy, an ophthalmic disorder or migraine, comprising administering an effective amount of a compound of the present invention to an animal in need of such treatment.

In some embodiments of the inventive method, the cardiovascular disease is heart failure, hypertension, SA/AV node disturbance, arrhythmia, hypertrophic subaortic stenosis or angina. In other embodiments of the inventive method, the heart failure is chronic heart failure or congestive heart failure.

The present invention further provides a method for inhibiting a β-adrenergic receptor and/or PDE, including PDE-3, comprising administering an effective amount of a compound of the present invention to an animal in need of such treatment.

The compound of the present invention may be administered by any means known to an ordinarily skilled artisan. For example, the compound of the present invention may be administered orally, parenterally, by inhalation spray, topically, rectally, nasally, buccally, vaginally, or via an implanted reservoir. The term “parenteral” as used herein includes subcutaneous, intravenous, intramuscular, intraperitoneal, intrathecal, intraventricular, intrasternal, intracranial, and intraosseous injection and infusion techniques. The exact administration protocol will vary depending upon various factors including the age, body weight, general health, sex and diet of the patient; the determination of specific administration procedures would be routine to an ordinarily skilled artisan.

The compound of the present invention may be administered by a single dose, multiple discrete doses or continuous infusion. Pump means, particularly subcutaneous pump means, are useful for continuous infusion.

Dose levels on the order of about 0.001 mg/kg/d to about 10,000 mg/kg/d of the compound of the present invention are useful for the inventive methods, with preferred levels being about 0.1 mg/kg/d to about 1,000 mg/kg/d, and more preferred levels being about 1 mg/kg/d to about 100 mg/kg/d. The specific dose level for any particular patient will vary depending upon various factors, including the activity and the possible toxicity of the specific compound employed; the age, body weight, general health, sex and diet of the patient; the time of administration; the rate of excretion; the drug combination; the severity of the congestive heart failure; and the form of administration. Typically, in vitro dosage-effect results provide useful guidance on the proper doses for patient administration. Studies in animal models are also helpful. The considerations for determining the proper dose levels are well known in the art and within the skill of a physician.

Any administration regimen well known to an ordinarily skilled artisan for regulating the timing and sequence of drug delivery can be used and repeated as necessary to effect treatment in the inventive method. The regimen may include pretreatment and/or co-administration with additional therapeutic agents.

The compound of the present invention can be administered alone or in combination with one or more additional therapeutic agent(s) for simultaneous, separate, or sequential use. The additional agent(s) may be any therapeutic agent(s), including without limitation one or more compound(s) of the present invention. The compound of the present invention can be co-administered with one or more therapeutic agent(s) either (i) together in a single formulation, or (ii) separately in individual formulations designed for optimal release rates of their respective active agent.

### Pharmaceutical Compositions

This invention further provides a pharmaceutical composition comprising a compound of the present invention. In one embodiment, the pharmaceutical composition comprises:

(i) an effective amount of a compound of the present invention; and

(ii) a pharmaceutically acceptable carrier.

The inventive pharmaceutical composition may comprise one or more additional pharmaceutically acceptable ingredient(s), including without limitation one or more wetting agent(s), buffering agent(s), suspending agent(s), lubricating agent(s), emulsifier(s), disintegrant(s), absorbent(s), preservative(s), surfactant(s), colorant(s), flavorant(s), sweetener(s) and additional therapeutic agent(s).

The inventive pharmaceutical composition may be formulated for administration in solid or liquid form, including those adapted for the following: (1) oral administration, for example, drenches (for example, aqueous or non-aqueous solutions or suspensions), tablets (for example, those targeted for buccal, sublingual and systemic absorption), boluses, powders, granules, pastes for application to the tongue, hard gelatin capsules, soft gelatin capsules, mouth sprays, emulsions and microemulsions; (2) parenteral administration, for example, by subcutaneous, intramuscular, intravenous or epidural injection as, for example, a sterile solution or suspension, or a sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

### EXAMPLES

### Example 1

Compounds 8a-8r were synthesized according to Scheme 1.

Scheme 1

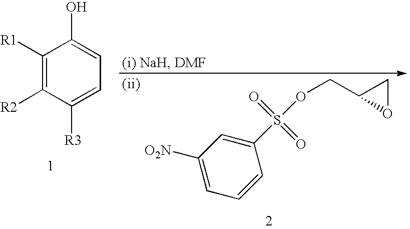

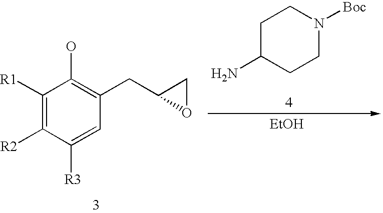

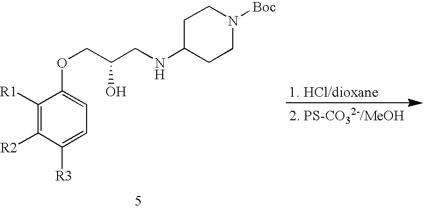

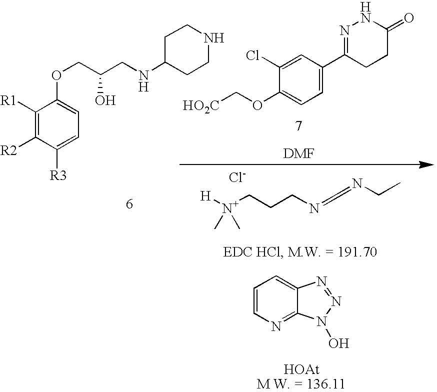

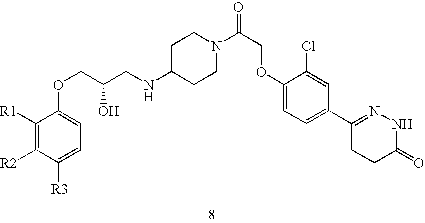

| Compound | R1 | R2 | R3 | M.W. of 8 |
|---|---|---|---|---|
| a | —H | —H | —H | 515.01 |
| b | —F | —H | —H | 533.00 |
| c | —Cl | —H | —H | 549.46 |
| d | —Br | —H | —H | 593.91 |
| e | —CN | —H | —H | 540.02 |
| f | -Me | —H | —H | 529.04 |
| g | —CF₃ | —H | —H | 583.01 |
| h | —OMe | —H | —H | 545.04 |
| i | —OCF₃ | —H | —H | 599.01 |
| j | -allyl | —H | —H | 555.08 |
| k | —COMe | —H | —H | 557.05 |
| l | —H | —Br | —H | 593.91 |
| m | —H | —CN | —H | 540.02 |
| n | —H | —H | —Br | 593.91 |
| o | —H | —H | —CN | 540.02 |
| p | —H | —H | -Me | 529.04 |
| q | —H | —H | —OMe | 545.04 |
| r | —H | —H | —CH₂CH₂OMe | 573.09 |

### Synthesis of (S)-2-Phenoxymethyl-oxirane (3a)

To a stirred suspension of sodium hydride (60% dispersion in mineral oil, 126 mg, 3.2 mmol) in N,N-dimethylformamide (4 mL) under N₂ at 0° C. was added portionwise a solution of phenol (1a, 282 mg, 3.0 mmol) in N,N-dimethylformamide (1 mL) and the reaction mixture was stirred at ambient temperature for 20 min. A solution of (2S)-glycidyl m-nitrobenzenesulfonate (2, 722 mg, 2.78 mmol) in N,N-dimethylformamide (2 mL) was then added at 0° C. The reaction mixture was stirred at ambient temperature for 16 h, poured onto a mixture of ice-water (15 mL) and saturated aqueous ammonium chloride solution (15 mL) and extracted with tert-butylmethyl ether (3×15 mL). The combined organic layers were washed with aqueous 1N sodium hydroxide solution (2×30 mL), 50% aqueous saturated brine (2×30 mL), and saturated brine (30 ml), dried (Na₂SO₄) and concentrated under reduced pressure to give (S)-2-phenoxymethyl-oxirane (3a) as a light yellow viscous oil (440 mg, 95% yield, >95% pure by LC-MS and ¹H-NMR).

### Synthesis of 4-((S)-2-hydroxy-3-phenoxy-propylamino)-piperidine-1-carboxylic acid tert-butyl ester (5a)

To a stirred solution of 2-phenoxymethyl-oxirane (3a, 440 mg, 2.65 mmol) in ethanol (15 mL) at ambient temperature was added 4-amino-piperidine-1-carboxylic acid tert-butyl ester (4, 1.5 g, 7.49 mmol). The reaction mixture was heated to 90° C., stirred at this temperature for 4 h and then concentrated under reduced pressure. The light yellow oily residue was purified by flash column chromatography over silica gel eluting with dichloro-methane/methanol (19:1). Fractions with R_{f}=0.28 (dichloromethane/methanol 9:1) were combined and concentrated under reduced pressure to give 4-(2-hydroxy-3-phenoxy-propylamino)-piperidine-1-carboxylic acid tert-butyl ester (5a) as a colorless, highly viscous oil (757 mg, 72% yield, 98% pure by LC-MS and ¹H-NMR).

### Synthesis of (S)-1-Phenoxy-3-(piperidin-4-ylamino)-propan-2-ol (6a)

To a stirred solution of 4-(2-hydroxy-3-phenoxy-propylamino)-piperidine-1-carboxylic acid tert-butyl ester (5a, 701 mg, 2.0 mmol) in methanol (5 mL) was added a solution of 4N hydrogen chloride in 1,4-dioxane (20 mL). The reaction mixture was stirred at ambient temperature for 2 h and then concentrated under reduced pressure. The residue was re-dissolved in methanol (40 mL) and Ambersep900-carbonate (13.3 g, 20 mmol) was added. The mixture was shaken at ambient temperature for 2 h, filtered and the filtrate concentrated under reduced pressure to give 1-phenoxy-3-(piperidin-4-ylamino)-propan-2-ol (6a) as a colorless highly viscous oil (454 mg, 92% yield, >90% pure by LC-MS and ¹H-NMR).

### Synthesis of 6-(3-Chloro-4-[2-[4-((S)-2-hydroxy-3-phenoxy-propylamino)-piperidin-1-yl]-2-oxo-ethoxy)-phenyl)-4,5-dihydro-2H-pyridazin-3-one (8a)

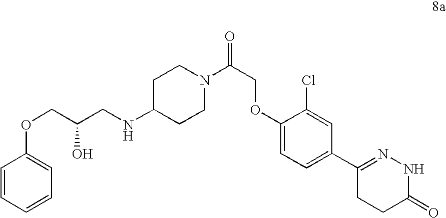

To a stirred solution of [2-chloro-4-(6-oxo-1,4,5,6-tetrahydro-pyridazin-3-yl)-phenoxy]-acetic acid (7, 375 mg, 1.3 mmol), 1-(3-dimethylaminopropyl)-3-ethyl carbodiimide hydrochloride (EDC.HCl, 562 mg, 2.9 mmol) and 7-hydroxyazabenzotriazole (HOAt, 225 mg, 1.6 mmol) in N,N-dimethylformamide (2 mL) at ambient temperature under nitrogen atmosphere was added a solution of 1-phenoxy-3-(piperidin-4-ylamino)-propan-2-ol (6a, 410 mg, 1.5 mmol) in N,N-dimethylformamide (3 mL). The reaction mixture was stirred at ambient temperature for 3 h then poured into 50% saturated brine (30 mL), adjusted to pH 10-11 using aqueous 2N sodium hydroxide and extracted with ethyl acetate (4×30 mL). The combined organic layers were washed with 50% saturated brine (4×70 mL), saturated brine (100 ml), dried (Na₂SO₄) and concentrated under reduced pressure. The residue was purified by flash column chromatography over silica gel (10 g) eluting with dichloromethane/methanol (9:1). Fractions with R_{f}=0.25 were combined and concentrated under reduced pressure to give 6-(3-chloro-4-{2-[4-(2-hydroxy-3-phenoxy-propylamino)-piperidin-1-yl]-2-oxo-ethoxy}-phenyl)-4,5-dihydro-2H-pyridazin-3-one (8a) as a colorless powder (390 mg, 57% yield, 98% pure by LC-MS and ¹H-NMR).

### Synthesis of 6-[3-Chloro-4-(2-[4-[(S)-3-(2-fluoro-phenoxy)-2-hydroxy-propyl-amino]-piperidin-1-yl}-2-oxo-ethoxy)-phenyl]-4,5-dihydro-2H-pyridazin-3-one (8b)

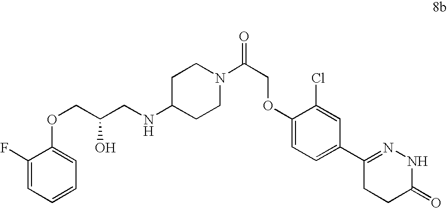

The same procedure as described for the synthesis of 8a was followed starting from 2-fluoro phenol (1b, 172 mg, 1.53 mmol). 6-[3-Chloro-4-(2-{4-[3-(2-fluoro-phenoxy)-2-hydroxy-propylamino]-piperidin-1-yl}-2-oxo-ethoxy)-phenyl]-4,5-dihydro-2H-pyridazin-3-one (8b) was isolated as a colorless gum (177 mg, 21% overall yield, 96% pure by LC-MS and ¹H-NMR).

### Synthesis of 6-[3-Chloro-4-(2-{4-[(S)-3-(2-chloro-phenoxy)-2-hydroxy-propylamino]-piperidin-1-yl}-2-oxo-ethoxy)-phenyl]-4,5-dihydro-2H-pyridazin-3-one (8c)

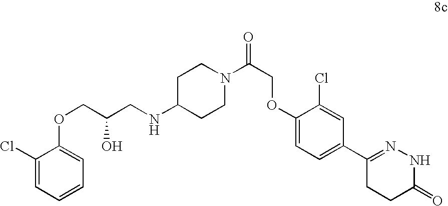

The same procedure as described for the synthesis of 8a was followed starting from 2-chloro phenol (1c, 262 mg, 2.04 mmol). 6-[3-Chloro-4-(2-{4-[3-(2-chloro-phenoxy)-2-hydroxy-propylamino]-piperidin-1-yl}-2-oxo-ethoxy)-phenyl]-4,5-dihydro-2H-pyridazin-3-one (8c) was isolated as a pale yellow powder (199 mg, 18% overall yield, 97% pure by LC-MS and ¹H-NMR). 99% pure by 10 min. LCMS (UV @215 nm: retention time=3.91 min., peak area=99%, TOF-ES⁺ with 25 eV cone voltage: m/z=549.16 (100%) & 551.14 (75%)). ¹H NMR: (CDCl₃, TMS internal standard, δ in ppm): 8.80 (1H, s), 7.80 (1H, s), 7.53 (1H, dd, J¹=8.56 Hz, J²=2.20 Hz), 7.35 (1H, d, J=7.83 Hz), 7.21 (1H, m), 7.03 (1H, d, J=8.56 Hz), 6.92 (2H, m), 4.82 (2H, d, J=6.60 Hz), 4.38 (1H, d, J=13.45 Hz), 4.12-4.00 (4H, m), 3.17 (1H, t, J=11.13 Hz), 2.98-2.69 (5H, m), 2.59 (2H, t, J=8.31 Hz), 2.03-1.88 (2H, m), 1.41-1.19 (2H, m).

### Synthesis of 6-[4-(2-{4-[(S)-3-(2-Bromo-phenoxy)-2-hydroxy-propylamino]-piperidin-1-yl}-2-oxo-ethoxy)-3-chloro-phenyl]-4,5-dihydro-2H-pyridazin-3-one (8d)

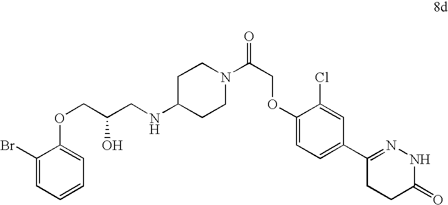

The same procedure as described for the synthesis of 8a was followed starting from 2-bromo phenol (1d, 355 μL, 1.38 mmol). 6-[4-(2-{4-[3-(2-Bromo-phenoxy)-2-hydroxy-propylamino]-piperidin-1-yl}-2-oxo-ethoxy)-3-chloro-phenyl]-4,5-dihydro-2H-pyridazin-3-one (8d) was isolated as a pale yellow powder (338 mg, 41% overall yield, 99% pure by LC-MS and ¹H-NMR). 99% pure by 10 min. LCMS (UV @215 nm: retention time=4.44 min., peak area=99%, TOF-ES⁺ with 25 eV cone voltage: m/z=593.25 (70%), 595.22 (100%) & 597.22 (30%)). ¹H NMR: (CDCl₃, TMS internal standard, δ in ppm): 8.72 (1H, s), 7.80 (1H, d, J=1.47 Hz), 7.53 (2H, m), 7.26 (1H, m), 7.03 (1H, d, J=8.80 Hz), 6.93-6.83 (2H, m), 4.82 (2H, m), 4.38 (1H, d, J=13.45 Hz), 4.06 (4H, m), 3.17 (1H, m), 2.99-2.70 (6H, m), 2.60 (2H, t, J=8.19 Hz), 1.96 (2H, m), 1.40-1.20 (2H, m).

### Synthesis of 2-[3-(1-{2-[(S)-2-Chloro-4-(6-oxo-1,4,5,6-tetrahydro-pyridazin-3-yl)-phenoxy]-acetyl}-piperidin-4-ylamino)-2-hydroxy-propoxy]-benzonitrile (8e)

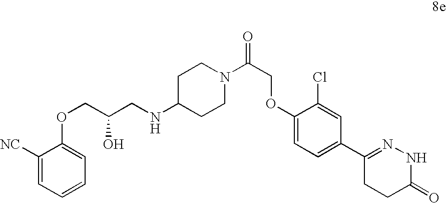

The same procedure as described for the synthesis of 8a was followed starting from 2-cyano phenol (1e, 449 mg, 3.77 mmol). 2-[3-(1-{2-[2-Chloro-4-(6-oxo-1,4,5,6-tetrahydro-pyridazin-3-yl)-phenoxy]-acetyl}-piperidin-4-ylamino)-2-hydroxy-propoxy]-benzonitrile (8e) was isolated as a pale yellow powder (211 mg, 10% overall yield, 98% pure by LC-MS and ¹H-NMR).

### Synthesis of 6-(3-Chloro-4-{2-[(S)-4-(2-hydroxy-3-o-tolyloxy-propylamino)-piperidin-1-yl]-2-oxo-ethoxyl-phenyl)-4,5-dihydro-2H-pyridazin-3-one (8f)

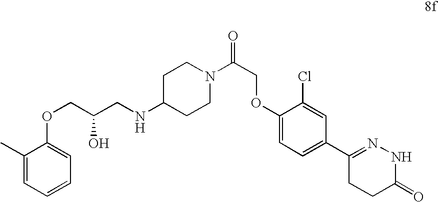

The same procedure as described for the synthesis of 8a was followed starting from 2-methyl phenol (1f, 216 mg, 2.0 mmol). 6-(3-Chloro-4-{2-[4-(2-hydroxy-3-o-tolyloxy-propylamino)-piperidin-1-yl]-2-oxo-ethoxy}-phenyl)-4,5-dihydro-2H-pyridazin-3-one (8f) was isolated as a pale yellow powder (262 mg, 25% overall yield, 98% pure by LC-MS and ¹H-NMR). 99% pure by 10 min. LCMS (UV @215 nm: retention time=3.95 min., peak area=99%, TOF-ES⁺ with 25 eV cone voltage: m/z=529.22 (100%) & 531.20 (35%)). ¹H NMR: (CDCl₃, TMS internal standard, δ in ppm): 8.89 (1H, s), 7.80 (1H, s), 7.53 (1H, dd, J¹=8.56 Hz, J²=2.20 Hz), 7.15 (2H, m), 7.03 (1H, d, J=8.80 Hz), 6.91-6.78 (2H, m), 4.82 (2H, m), 4.39 (1H, d, J=13.45 Hz), 4.11-3.94 (4H, m), 3.17 (1H, t, J=11.74 Hz), 2.98-2.70 (6H, m), 2.59 (2H, m), 2.22 (3H, s), 1.96 (2H, m), 1.40-1.18 (2H, m).

### Synthesis of 6-[3-Chloro-4-(2-{4-[(S)-2-hydroxy-3-(2-trifluoromethyl-phenoxy)-propylamino]-piperidin-1-yl}-2-oxo-ethoxy)-phenyl]-4,5-dihydro-2H-pyridazin-3-one (8g)

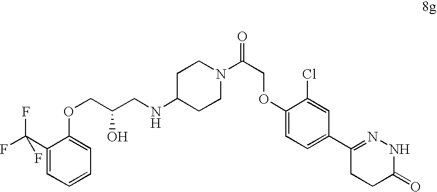

The same procedure as described for the synthesis of 8a was followed starting from 2-trifluoromethyl phenol (1 g, 331 mg, 2.04 mmol). 6-[3-Chloro-4-(2-{4-[2-hydroxy-3-(2-trifluoromethyl-phenoxy)-propylamino]-piperidin-1-yl}-2-oxo-ethoxy)-phenyl]-4,5-dihydro-2H-pyridazin-3-one (8g) was isolated as a pale yellow powder (256 mg, 22% overall yield, 98% pure by LC-MS and ¹H-NMR). 99% pure by 10 min. LCMS (UV @215 nm: retention time=4.15 min., peak area=99%, TOF-ES⁺ with 25 eV cone voltage: m/z=583.19 (100%) & 585.18 (30%)). ¹H NMR: (CDCl₃, TMS internal standard, δ in ppm): 8.78 (1H, s), 7.80 (1H, d, J=2.20 Hz), 7.59-7.46 (3H, m), 7.07-6.96 (3H, m), 4.82 (2H, d, J=5.87 Hz), 4.38 (1H, d, J=13.45 Hz), 4.13-4.00 (4H, m), 3.17 (1H, t, J=11.25 Hz), 2.98-2.78 (5H, m), 2.73 (1H, m), 2.60 (2H, m), 1.95 (2H, m), 1.39-1.17 (2H, m).

### Synthesis of 6-[3-Chloro-4-(2-{4-[(S)-2-hydroxy-3-(2-methoxy-phenoxy)-propylamino]-piperidin-1-yl}-2-oxo-ethoxy)-phenyl-4,5-dihydro-2H-pyridazin-3-one (8h)

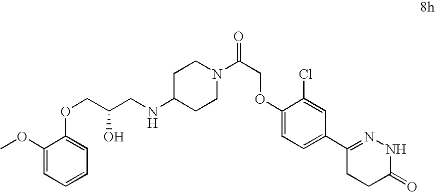

The same procedure as described for the synthesis of 8a was followed starting from 2-methoxy phenol (1h, 253 mg, 2.04 mmol). 6-[3-Chloro-4-(2-{4-[2-hydroxy-3-(2-methoxy-phenoxy)-propylamino]-piperidin-1-yl}-2-oxo-ethoxy)-phenyl]-4,5-dihydro-2H-pyridazin-3-one (8h) was isolated as a pale yellow powder (202 mg, 18% overall yield, 99% pure by LC-MS and ¹H-NMR). 99% pure by 10 min. LCMS (UV @215 nm: retention time=3.72 min., peak area=99%, TOF-ES⁺ with 25 eV cone voltage: m/z=545.24 (100%) & 547.19 (40%)). ¹H NMR: (CDCl₃, TMS internal standard, δ in ppm): 8.74 (1H, s), 7.80 (1H, d, J=2.20 Hz), 7.53 (1H, dd, J¹=8.56 Hz, J²=2.20 Hz), 7.03 (1H, d, J=8.56 Hz), 6.98-6.86 (4H, m), 4.82 (2H, m), 4.36 (1H, d, J=13.45 Hz), 4.10-3.96 (4H, m), 3.84 (3H, s), 3.18 (1H, t, J=11.49 Hz), 2.96-2.67 (6H, m), 2.60 (2H, t, J=8.19 Hz), 1.94 (2H, m), 1.40-1.19 (2H, m).

### Synthesis of 6-[3-Chloro-4-(2-{4-[(S)-2-hydroxy-3-(2-trifluoromethoxy-phenoxy)-propylamino]-piperidin-1-yl}-2-oxo-ethoxy)-phenyl]-4,5-dihydro-2H-pyridazin-3-one (8i)

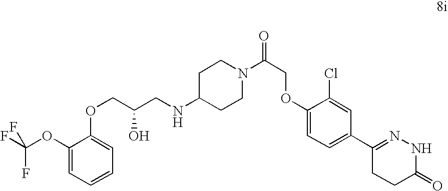

The same procedure as described for the synthesis of 8a was followed starting from 2-trifluoromethoxy phenol (1i, 267 mg, 1.50 mmol). 6-[3-Chloro-4-(2-{4-[2-hydroxy-3-(2-trifluoromethoxy-phenoxy)-propylamino]-piperidin-1-yl}-2-oxo-ethoxy)-phenyl]-4,5-dihydro-2H-pyridazin-3-one (8i) was isolated as a pale yellow powder (203 mg, 22% overall yield, 98% pure by LC-MS and ¹H-NMR).

### Synthesis of 6-[4-(2-{4-[(S)-3-(2-Allyl-phenoxy)-2-hydroxy-propylamino]-piperidin-1-yl}-2-oxo-ethoxy)-3-chloro-phenyl]-4,5-dihydro-2H-pyridazin-3-one (8j)

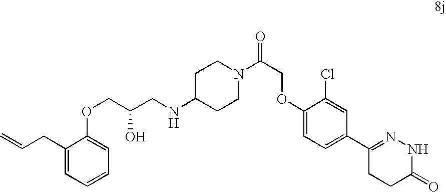

The same procedure as described for the synthesis of 8a was followed starting from 2-allyl phenol (1j, 274 mg, 2.04 mmol). 6-[4-(2-{4-[3-(2-Allyl-phenoxy)-2-hydroxy-propylamino]-piperidin-1-yl}-2-oxo-ethoxy)-3-chloro-phenyl]-4,5-dihydro-2H-pyridazin-3-one (8j) was isolated as a pale yellow powder (167 mg, 15% overall yield, 99% pure by LC-MS and ¹H-NMR). 99% pure by 10 min. LCMS (UV @215 nm: retention time=4.20 min., peak area=99%, TOF-ES⁺ with 25 eV cone voltage: m/z=555.24 (100%) & 557.21 (35%)). ¹H NMR: (CDCl₃, TMS internal standard, δ in ppm): 8.92 (1H, s), 7.80 (1H, d, J=2.20 Hz), 7.53 (1H, dd, J¹=8.80 Hz, J²=2.20 Hz), 7.22-7.14 (2H, m), 7.03 (1H, d, J=8.80 Hz), 6.92 (1H, t, J=7.34 Hz), 6.84 (1H, d, J=7.83 Hz), 6.03-5.91 (1H, m), 5.02 (2H, m), 4.82 (2H, m), 4.38 (1H, d, J=13.20 Hz), 4.07 (2H, m), 3.98 (2H, m), 3.38 (2H, d, J=5.14 Hz), 3.17 (1H, t, J=11.49 Hz), 2.96-2.69 (6H, m), 2.59 (2H, t, J=8.31 Hz), 1.95 (2H, m), 1.39-1.19 (2H, m).

### Synthesis of 6-[4-(2-{4-[(S)-3-(2-Acetyl-phenoxy)-2-hydroxy-propylamino]-piperidin-1-yl}-2-oxo-ethoxy)-3-chloro-phenyl]-4,5-dihydro-2H-pyridazin-3-one (8k)

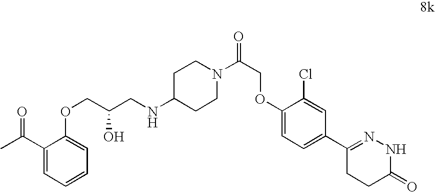

The same procedure as described for the synthesis of 8a was followed starting from 2-hydroxy acetophenone (1k, 408 mg, 3.00 mmol). 6-[4-(2-{4-[3-(2-Acetyl-phenoxy)-2-hydroxy-propylamino]-piperidin-1-yl}-2-oxo-ethoxy)-3-chloro-phenyl]-4,5-dihydro-2H-pyridazin-3-one (8k) was isolated as a light yellow powder (183 mg, 11% overall yield, 99% pure by LC-MS and ¹H-NMR).

### Synthesis of 6-[4-(2-{4-[(S)-3-(3-Bromo-phenoxy)-2-hydroxy-propylamino]-piperidin-1-yl}-2-oxo-ethoxy)-3-chloro-phenyl]-4,5-dihydro-2H-pyridazin-3-one (8l)

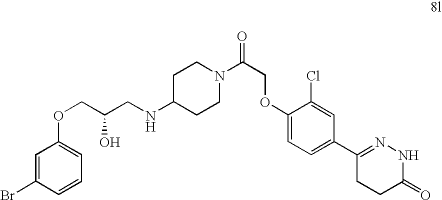

The same procedure as described for the synthesis of 8a was followed starting from 3-bromo phenol (1l, 530 mg, 3.06 mmol). 6-[4-(2-{4-[3-(3-Bromo-phenoxy)-2-hydroxy-propylamino]-piperidin-1-yl}-2-oxo-ethoxy)-3-chloro-phenyl]-4,5-dihydro-2H-pyridazin-3-one (8l) was isolated as a light yellow powder (149 mg, 8% overall yield, 96% pure by LC-MS and ¹H-NMR). 96% pure by 10 min. LCMS (UV @215 nm: retention time=4.62 min., peak area=96%, TOF-ES⁺ with 25 eV cone voltage: m/z=593.25 (70%), 595.24 (100%) & 597.25 (25%)). ¹H NMR: (CDCl₃, TMS internal standard, δ in ppm): 8.71 (1H, s), 7.80 (1H, d, J=2.20 Hz), 7.53 (1H, dd, J¹=8.80 Hz, J²=2.20 Hz), 7.19-7.02 (4H, m), 6.84 (1H, m), 4.82 (2H, m), 4.39 (1H, d, J=13.20 Hz), 4.12-3.93 (4H, m), 3.17 (1H, t, J=11.25 Hz), 2.96-2.69 (6H, m), 2.60 (2H, t, J=8.31 Hz), 1.95 (2H, m), 1.41-1.18 (2H, m).

### Synthesis of 3-[3-(1-{2-[(S)-2-Chloro-4-(6-oxo-1,4,5,6-tetrahydro-pyridazin-3-yl)-phenoxy]-acetyl}-piperidin-4-ylamino)-2-hydroxy-propoxy]-benzonitrile (8m)

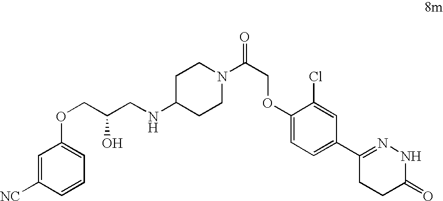

The same procedure as described for the synthesis of 8a was followed starting from 3-cyano phenol (1m, 243 mg, 2.04 mmol). 3-[3-(1-{2-[2-Chloro-4-(6-oxo-1,4,5,6-tetrahydro-pyridazin-3-yl)-phenoxy]-acetyl}-piperidin-4-ylamino)-2-hydroxy-propoxy]-benzonitrile (8m) was isolated as a light yellow powder (201 mg, 18% overall yield, 99% pure by LC-MS and ¹H-NMR). 99% pure by 10 min. LCMS (UV @215 nm: retention time=3.65 min., peak area=99%, TOF-ES⁺ with 25 eV cone voltage: m/z=540.26 (30%) & 542.29 (15%)). ¹H NMR: (CDCl₃, TMS internal standard, δ in ppm): 8.69 (1H, s), 7.81 (1H, d, J=2.20 Hz), 7.55 (3H, m), 7.03 (3H, m), 4.83 (2H, m), 4.40 (1H, d, J=13.26 Hz), 4.17-4.00 (4H, m), 3.18 (1H, m), 2.94 (3H, t, J=8.19 Hz), 2.88-2.69 (3H, m), 2.60 (2H, t, J=8.23 Hz), 2.07-1.88 (2H, m), 1.41-1.19 (2H, m).

### Synthesis of 6-[4-(2-{4-[(S)-3-(4-Bromo-phenoxy)-2-hydroxy-propylamino]-piperidin-1-yl}-2-oxo-ethoxy)-3-chloro-phenyl]-4,5-dihydro-2H-pyridazin-3-one (8n)

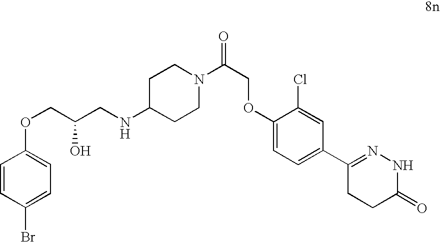

The same procedure as described for the synthesis of 8a was followed starting from 4-bromo phenol (1n, 262 mg, 1.51 mmol). 6-[4-(2-{4-[3-(4-Bromo-phenoxy)-2-hydroxy-propylamino]-piperidin-1-yl}-2-oxo-ethoxy)-3-chloro-phenyl]-4,5-dihydro-2H-pyridazin-3-one (8n) was isolated as a light yellow powder (130 mg, 14% overall yield, 99% pure by LC-MS and ¹H-NMR).

### Synthesis of 4-[3-(1-{2-[(S)-2-Chloro-4-(6-oxo-1,4,5,6-tetrahydro-pyridazin-3-yl)-phenoxy]-acetyl}-piperidin-4-ylamino)-2-hydroxy-propoxyl-benzonitrile (8o)

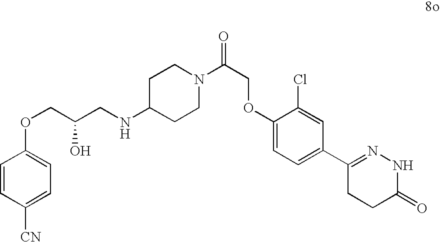

The same procedure as described for the synthesis of 8a was followed starting from 4-cyano phenol (1o, 188 mg, 1.58 mmol). 4-[3-(1-{2-[2-Chloro-4-(6-oxo-1,4,5,6-tetrahydro-pyridazin-3-yl)-phenoxy]-acetyl}-piperidin-4-ylamino)-2-hydroxy-propoxy]-benzonitrile (8o) was isolated as a light yellow powder (74 mg, 9% overall yield, 98% pure by LC-MS and ¹H-NMR).

### Synthesis of 6-(3-Chloro-4-{2-[(S)-4-(2-hydroxy-3-p-tolyloxy-propylamino)-piperidin-1-yl]-2-oxo-ethoxyl-phenyl)-4,5-dihydro-2H-pyridazin-3-one (8p)

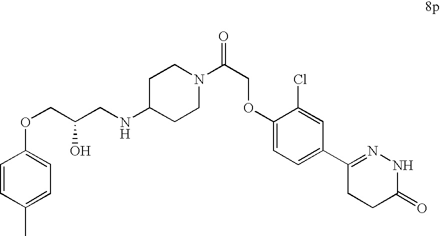

The same procedure as described for the synthesis of 8a was followed starting from 4-methyl phenol (1p, 164 mg, 1.52 mmol). 6-(3-Chloro-4-{2-[4-(2-hydroxy-3-p-tolyloxy-propylamino)-piperidin-1-yl]-2-oxo-ethoxy}-phenyl)-4,5-dihydro-2H-pyridazin-3-one (8p) was isolated as a light yellow powder (168 mg, 21% overall yield, 99% pure by LC-MS and ¹H-NMR).

### Synthesis of 6-[3-Chloro-4-(2-{4-[(S)-2-hydroxy-3-(4-methoxy-phenoxy)-propylamino]-piperidin-1-yl}-2-oxo-ethoxy)-phenyl]-4,5-dihydro-2H-pyridazin-3-one (8q)

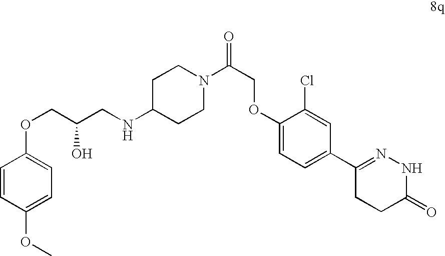

The same procedure as described for the synthesis of 8a was followed starting from 4-methoxy phenol (1q, 188 mg, 1.51 mmol). 6-[3-Chloro-4-(2-{4-[2-hydroxy-3-(4-methoxy-phenoxy)-propylamino]-piperidin-1-yl}-2-oxo-ethoxy)-phenyl]-4,5-dihydro-2H-pyridazin-3-one (8q) was isolated as a light yellow powder (95 mg, 12% overall yield, 99% pure by LC-MS and ¹H-NMR).

### Synthesis of 6-{3-Chloro-4-[(S)-2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (8r)

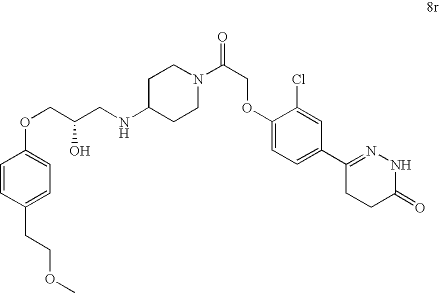

The same procedure as described for the synthesis of 8a was followed starting from 4-(2′-methoxy)ethyl phenol (1r, 465 mg, 3.05 mmol). 6-{3-Chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (8r) was isolated as a light yellow powder (319 mg, 18% overall yield, 98% pure by LC-MS and ¹H-NMR).

### Example 2

6-{3-Chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (13) was synthesized according to Scheme 2.

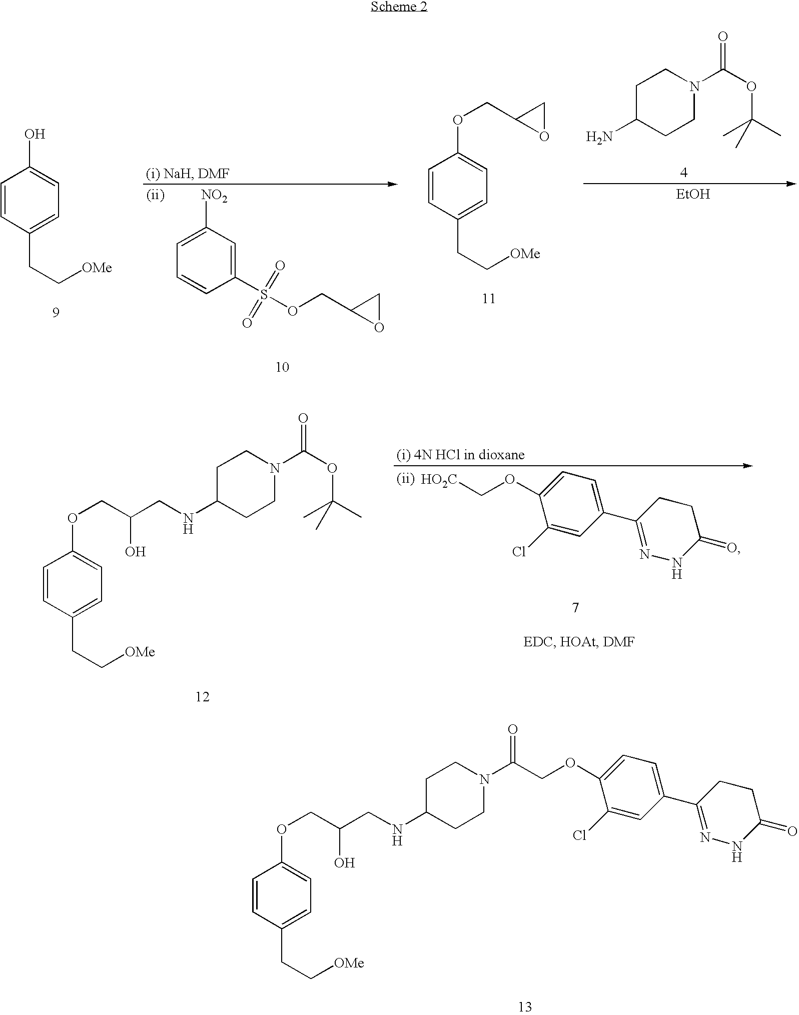

### Synthesis of 6-{3-Chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (13)

Compound 13 was synthesized via the procedure described for 8r but using racemic 3-nitro-benzenesulfonic acid oxiranylmethyl ester (10) instead of the enantiomerically pure material (2) in the first reaction step. A colorless powder (320 mg, 78% yield over last 2 steps), 99% pure by LC-MS and ¹H-nmr was obtained. 10 min LC-MS (UV @215 nm: retention time=3.90 min., peak area=100%, TOF-ES⁺ with 25 eV cone voltage: m/z=573 (100%) & 575 (50%)). ¹H NMR: (CDCl₃, TMS internal standard, δ in ppm): 8.66 (1H, s), 7.80 (1 H, d, J=2.20 Hz), 7.53 (1H, dd, J¹=8.80 Hz, J²=2.20 Hz), 7.13 (2H, d, J=8.56 Hz), 7.03 (1H, d, J=8.56 Hz), 6.83 (2H, d, J=8.56 Hz), 4.82 (2H, m), 4.38 (1H, d, J=13.45 Hz), 4.11-3.98 (2H, m), 3.95 (2H, m), 3.56 (2H, t, J=6.97 Hz), 3.35 (3H, s), 3.17 (1H, m), 2.96-2.85 (3H, m), 2.85-2.70 (4H, m), 2.60 (2H, m), 1.95 (2H, m), 1.38-1.18 (2H, m).

### Example 3

SYNTHESIS OF 6-[4-(2-{4-[(S)-3-(9H-CARBAZOL-4-YLOXY)-2-HYDROXY-PROPYLAMINO]-PIPERIDIN-1-YL}-2-OXO-ETHOXY)-3-CHLORO-PHENYL]-4,5-DIHYDRO-2H-PYRIDAZIN-3-ONE (17)

6-[4-(2-{4-[(S)-3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (17) was synthesized according to Scheme 3.

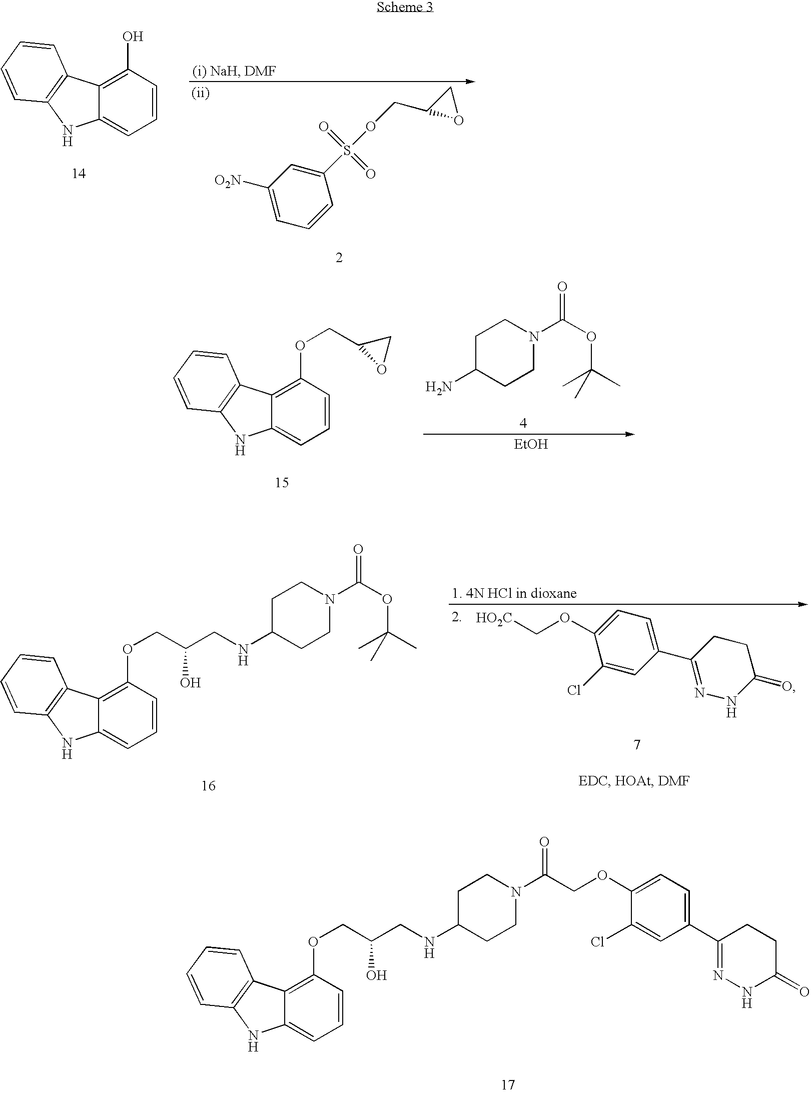

### Synthesis of 4-((S)-Oxiranylmethoxy)-9H-carbazole (15)

To a stirred solution of 9H-carbazol-4-ol (14, 750 mg, 4.09 mmol) and (2S)-glycidyl m-nitrobenzenesulfonate (2, 1061 mg, 4.09 mmol) in butan-2-one (40 mL) was added potassium carbonate (622 mg, 4.50 mmol). The mixture was stirred under reflux for 18 h and the solvent was then removed under reduced pressure. The residue was re-dissolved in dichloromethane (150 mL) and the solution was washed with aqueous sodium hydroxide (1N, 2×50 mL), water (50 mL) and saturated brine (50 mL). The organic layer was dried over sodium sulphate and evaporated to dryness. 4-((S)-Oxiranylmethoxy)-9H-carbazole (15) was isolated as light brown solid (920 mg, 94% yield, 96% pure by LC-MS and ¹H-nmr).

### Synthesis of 4-[(S)-3-(9H-Carbazol-4-yloxy)-2-hydroxy-propylamino]-piperidine-1-carboxylic acid tert-butyl ester (16)

To a stirred suspension of 4-((S)-oxiranylmethoxy-9H-carbazole (15, 300 mg, 1.25 mmol) in ethanol (15 mL) was added 4-amino-piperidine-1-carboxylic acid tert-butyl ester (4, 502 mg, 2.81 mmol). The mixture was stirred for 4 h under reflux and the solvent was then removed under reduced pressure. The residue was purified by flash column chromatography on a Biotage® system (40-M column) eluting with a gradient of 1-5% methanol in dichloromethane. 4-[3-(9H-Carbazol-4-yloxy)-2-hydroxy-propylamino]-piperidine-1-carboxylic acid tert-butyl ester (16) was isolated as a brown gum (312 mg, 57% yield, 92% pure by LC-MS and ¹H-nmr).

### Synthesis of 6-[4-(2-{4-[(S)-3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (17)

To a stirred solution of 4-[(S)-3-(9H-carbazol-4-yloxy)-2-hydroxy-propylamino]-piperidine-1-carboxylic acid tert-butyl ester (16, 312 mg, 0.709 mmol) in methanol (5 mL) was added hydrogen chloride in 1,4-dioxane (4N, 2.5 mL, 10.0 mmol). The mixture was stirred at ambient temperature for 16 h and the solvent was then removed under reduced pressure. The residue was dissolved in methanol (30 mL) and Ambersep® 900 (carbonate form, 2.5 g, 3.75 mmol) was added. The mixture was shaken at ambient temperature for 3 h and then filtered. The filter residue was rinsed with methanol (3×15 mL) and the combined filtrates were evaporated to dryness. The residue was re-dissolved in N,N-dimethylformamide (4 mL). Separately, a suspension of (3-dimethylamino-propyl)-ethyl-carbodiimide hydrochloride (EDC.HCl, 270 mg, 1.41 mmol), [1,2,3]triazolo[4,5-b]pyridin-3-ol (HOAt, 96 mg, 0.70 mmol) and [2-chloro-4-(6-oxo-1,4,5,6-tetrahydro-pyridazin-3-yl)-phenoxy]-acetic acid (7, 199 mg, 0.70 mmol) in N,N-dimethylformamide (4 mL) was stirred at ambient temperature for 30 minutes until a clear solution. Both solutions were combined and the mixture was stirred for 16 h at ambient temperature then diluted with ethyl acetate (30 mL) and water (60 mL). The mixture was adjusted to pH=12 with 2N aqueous sodium hydroxide solution. The aqueous phase was extracted with ethyl acetate (3×30 mL). The combined organic layers were washed with saturated brine (4×100 mL), dried over sodium sulphate and evaporated to dryness. The residue was purified by flash column chromatography on silica gel (10 g) eluting with dichloromethane/methanol 10:1 to give 6-[4-(2-{4-[(S)-3-(9H-carbazol-4-yloxy)-2-hydroxy-propylamino]-piperidin-1-yl}-2-oxo-ethoxy)-3-chloro-phenyl]-4,5-dihydro-2H-pyridazin-3-one (17) as colorless powder (220 mg, 51% yield, 99% pure by LC-MS and ¹H-nmr). 2.5 min LC-MS (UV @215 nm: retention time=1.12 min., peak area=100%, TOF-ES⁺ with 25 eV cone voltage: m/z=604 (100%) & 606 (50%)). ¹H NMR: ([D₆]-DMSO, δ in ppm): 11.23 (1H, s), 10.88 (1H, s), 8.20 (1H, d, J=7.78 Hz), 7.76 (1H, d, J=2.29 Hz), 7.60 (1H, dd, J¹=8.74 Hz, J²=2.24 Hz), 7.43 (1H, d, J=8.05 Hz), 7.34-7.26 (2H, m), 7.12 (1H, m), 7.05 (1H, d, J=7.96 Hz), 7.02 (1H, d, J=8.87 Hz), 6.67 (1H, d, J=7.87 Hz), 5.11 (1H, m), 4.99 (2H, m), 4.15 (2H, m), 4.05 (2H, m), 3.74 (1H, m), 3.07 (1H, m), 2.89 (3H, m), 2.83-2.65 (3H, br m), 2.41 (2H, t, J=8.28 Hz), 1.82 (2H, m), 1.30 (1H, m), 1.13 (1H, m).

### Example 4

6-(4-{2-[3-(9H-carbazol-4-yloxy)-2-hydroxy-propylamino]-ethoxy}-3-chloro-phenyl)-4,5-dihydro-2H-pyridazin-3-one (22) was synthesized according to Scheme 4.

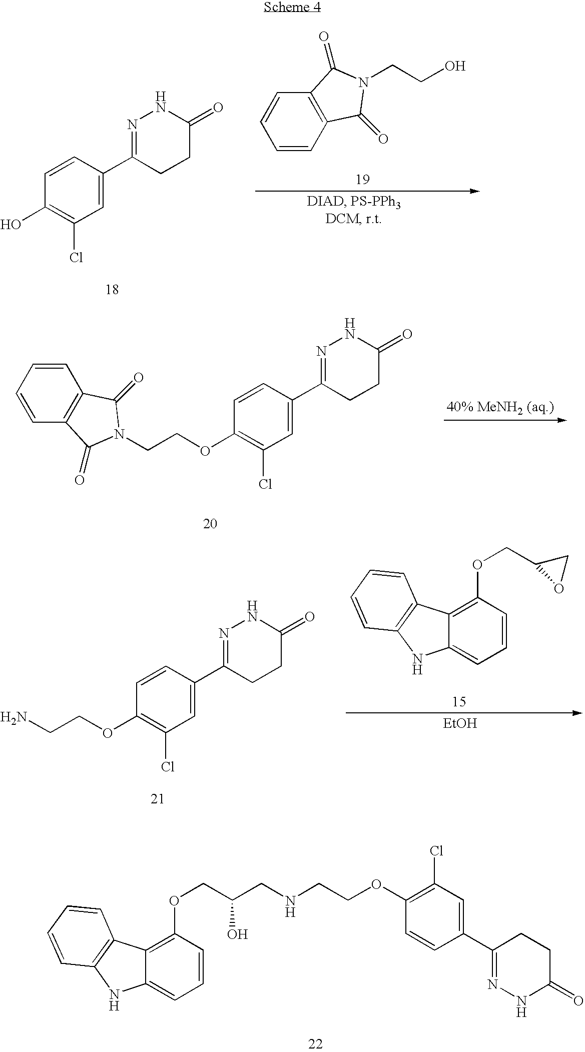

### Synthesis of 2-{2-[2-Chloro-4-(6-oxo-1,4,5,6-tetrahydro-pyridazin-3-yl)-phenoxy]-ethyl}-isoindole-1,3-dione (20)

A suspension of 6-(3-chloro-4-hydroxy-phenyl)-4,5-dihydro-2H-pyridazin-3-one (18, 500 mg, 2.22 mmol), 2-(2-hydroxy-ethyl)-isoindole-1,3-dione (19, 424 mg, 2.22 mmol) and polymer-supported triphenylphosphine (2.22 g, 3.33 mmol) in dichloromethane (30 mL) was vigorously stirred for 10 min at ambient temperature. To the suspension was added diisopropyl azodicarboxylate (530 μL, 2.67 mmol) in one portion. The mixture was stirred for 18 h at ambient temperature and then filtered. The filter residue was rinsed with dichloromethane (2×15 mL) and tetrahydrofuran (2×15 mL). The combined filtrates were evaporated to dryness. The residue was absorbed onto silica gel (1 g) from dichloromethane, dry-loaded onto a silica gel column (25 g) and purified by flash chromatography eluting with dichloromethane/ethyl acetate 2:1 to give 2-{2-[2-chloro-4-(6-oxo-1,4,5,6-tetrahydro-pyridazin-3-yl)-phenoxy]-ethyl}-isoindole-1,3-dione (20) as a colorless powder (375 mg, 42% yield, 97% pure by LC-MS and ¹H-nmr).

### Synthesis of 6-[4-(2-Amino-ethoxy)-3-chloro-phenyl]-4,5-dihydro-2H-pyridazin-3-one (21)

A suspension of 2-{2-[2-chloro-4-(6-oxo-1,4,5,6-tetrahydro-pyridazin-3-yl)-phenoxy]-ethyl}-isoindole-1,3-dione (20, 375 mg, 0.942 mmol) in aqueous methylamine (40 wt %, 5 mL) was stirred for 15 h at ambient temperature. The mixture was separated between dichloromethane (50 mL) and water (50 mL). The aqueous phase was extracted with dichloromethane (50 mL). The combined organic layers were washed with aqueous sodium hydroxide solution (1N, 50 mL) and saturated brine (50 mL), dried over sodium sulphate and evaporated to dryness to give 6-[4-(2-amino-ethoxy)-3-chloro-phenyl]-4,5-dihydro-2H-pyridazin-3-one (21) as colorless powder (225 mg, 89% yield, >90% pure by LC-MS and ¹H-nmr).

### Synthesis of 6-(4-{2-[3-(9H-carbazol-4-yloxy)-2-hydroxy-propylamino]-ethoxy}-3-chloro-phenyl)-4,5-dihydro-2H-pyridazin-3-one (22)

A suspension of 6-[4-(2-amino-ethoxy)-3-chloro-phenyl]-4,5-dihydro-2H-pyridazin-3-one (21, 225 mg, 0.840 mmol) and 4-((S)-oxiranylmethoxy-9H-carbazole (15, 134 mg, 0.560 mmol) in ethanol (40 mL) was stirred for 8 h under reflux. The solvent was removed under reduced pressure and the residue absorbed onto silica gel (500 mg) from dichloromethane/methanol 100:5. The material was dry-loaded onto a silica gel column (10 g) and purified by flash chromatography eluting with dichloromethane/methanol 20:1 to give 6-(4-{2-[(S)-3-(9H-carbazol-4-yloxy)-2-hydroxy-propylamino]-ethoxy}-3-chloro-phenyl)-4,5-dihydro-2H-pyridazin-3-one (22) as colorless powder (130 mg, 46% yield, 99% pure by LC-MS and ¹H-nmr). 2.5 min LC-MS (UV @215 nm: retention time=1.10 min., peak area=100%, TOF-ES⁺ with 25 eV cone voltage: m/z=507 (100%) & 509 (50%)). ¹H NMR: ([D₆]-DMSO, δ in ppm): 11.23 (1H, s), 10.88 (1H, s), 8.20 (1H, d, J=7.78 Hz), 7.75 (1H, d, J=2.20 Hz), 7.61 (1H, dd, J¹=8.69 Hz, J²=2.20 Hz), 7.42 (1H, d, J=8.05 Hz), 7.34-7.24 (2H, m), 7.16 (1H, d, J=8.78 Hz), 7.10 (1H, m), 7.05 (1H, d, J=7.96 Hz), 6.66 (1H, d, J=7.96 Hz), 5.16 (1H, br m), 4.14 (5H, br m), 3.01-2.92 (3H, m), 2.92-2.82 (3H, m), 2.41 (2H, t, J=8.23 Hz), 2.50-2.10 (1H, br).

### Example 5

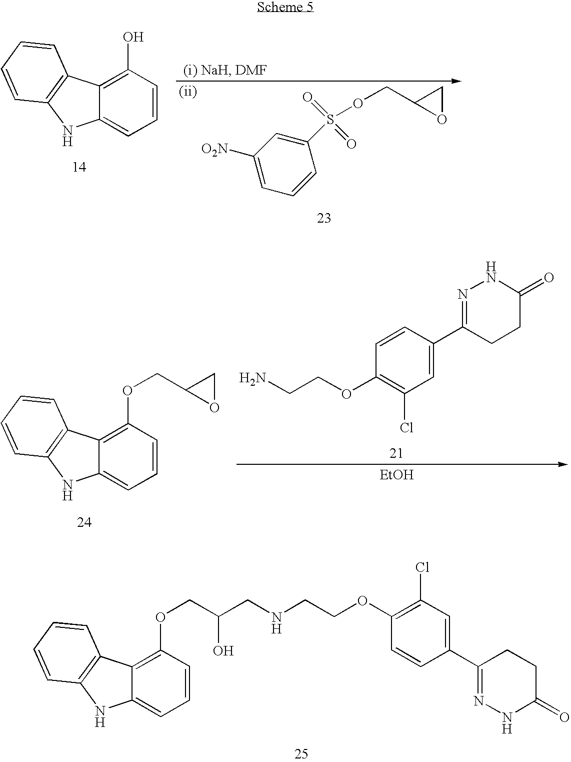

### Synthesis of 6-(4-{2-[3-(9H-Carbazol-4-yloxy)-2-hydroxy-propylamino]-ethoxy}-3-chloro-phenyl)-4,5-dihydro-2H-pyridazin-3-one (25)

Compound 25 was synthesized via the procedure described for 22 using racemic 3-nitro-benzenesulfonic acid oxiranylmethyl ester (23) instead of the enantiomerically pure material (2), as shown in Scheme 5. A colorless powder (136 mg, 48% yield, 99% pure by LC-MS and ¹H-nmr) was obtained. 2.5 min LC-MS (UV @215 nm: retention time=1.09 min., peak area=100%, TOF-ES⁺ with 25 eV cone voltage: m/z=507 (100%) & 509 (40%)). ¹H NMR: ([D₆]-DMSO, δ in ppm): 11.23 (1H, s), 10.88 (1H, s), 8.20 (1H, d, J=7.83 Hz), 7.75 (1H, d, J=1.96 Hz), 7.61 (1H, dd, J¹=8.80 Hz, J²=2.20 Hz), 7.42 (1H, d, J=8.07 Hz), 7.34-7.24 (2H, m), 7.15 (1H, d, J=8.80 Hz), 7.10 (1H, t, J=7.46 Hz), 7.05 (1H, d, J=8.07 Hz), 6.66 (1H, d, J=7.83 Hz), 5.16 (1H, br d, J=4.65 Hz), 4.15 (5H, br m), 3.01-2.92 (3H, m), 2.92-2.82 (3H, m), 2.41 (2H, t, J=8.31 Hz), 2.06 (1H, br s).

### Example 6

6-(4-{3-[(S)-3-(9H-Carbazol-4-yloxy)-2-hydroxy-propylamino]-propoxy}-3-chloro-phenyl)-4,5-dihydro-2H-pyridazin-3-one (29) was synthesized according to Scheme 6.

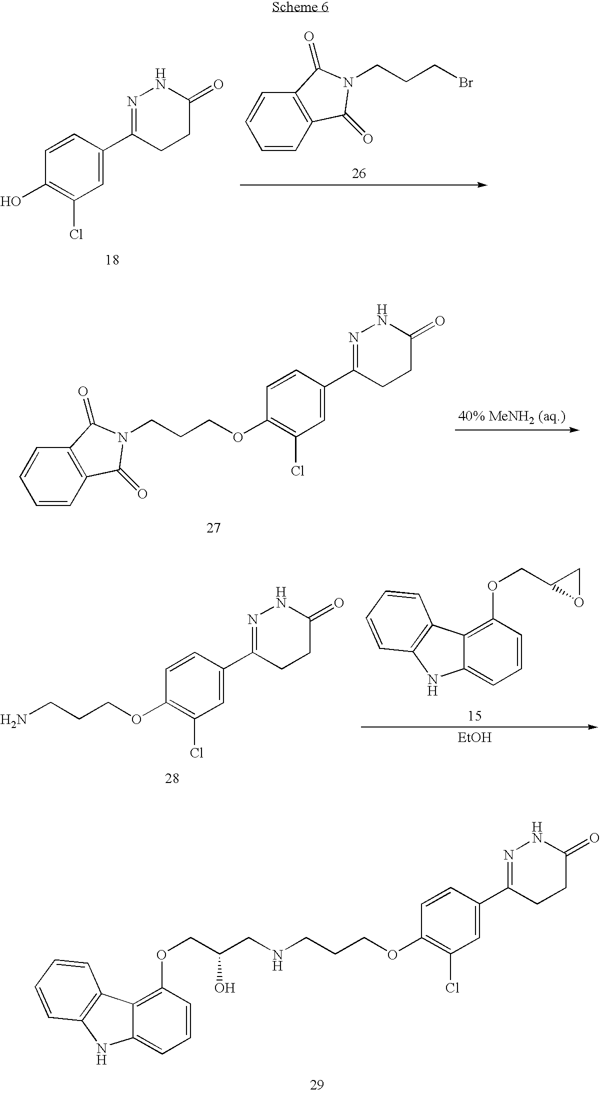

### Synthesis of 2-{3-[2-Chloro-4-(6-oxo-1,4,5,6-tetrahydro-pyridazin-3-yl)-phenoxy]-propyl}-isoindole-1,3-dione (27)

To a stirred solution of 6-(3-chloro-4-hydroxy-phenyl)-4,5-dihydro-2H-pyridazin-3-one (18, 1.00 g, 4.45 mmol) and 2-(3-bromo-propyl)-isoindole-1,3-dione (26, 1.19 g, 4.45 mmol) in N,N-dimethylformamide (10 mL) was added potassium carbonate (677 mg, 4.90 mg). The mixture was stirred for 18 h at ambient temperature and diluted with water (60 mL) to obtain a precipitate. The suspension was stirred for 20 min at ambient temperature. The precipitate was filtered off, rinsed with water (20 mL) and dried i. vac. to constant weight. 2-{3-[2-Chloro-4-(6-oxo-1,4,5,6-tetrahydro-pyridazin-3-yl)-phenoxy]-propyl}-isoindole-1,3-dione (27) was isolated as colorless powder (1.85 g, 92% yield, 96% pure by LC-MS and ¹H-nmr).

### Synthesis of 6-[4-(3-Amino-propoxy)-3-chloro-phenyl]-4,5-dihydro-2H-pyridazin-3-one (28)

A suspension of 2-{3-[2-Chloro-4-(6-oxo-1,4,5,6-tetrahydro-pyridazin-3-yl)-phenoxy]-propyl}-isoindole-1,3-dione (27, 1.85 g, 4.45 mmol) in aqueous methylamine (40 wt %, 60 mL) was stirred for 16 h at ambient temperature. The mixture was separated between dichloromethane (200 mL) and water (200 mL). The aqueous phase was extracted with dichloromethane (2×100 mL). The combined organic layers were washed with aqueous sodium hydroxide solution (1N, 200 mL) and saturated brine (200 mL), dried over sodium sulphate and evaporated to dryness. 6-[4-(3-Amino-propoxy)-3-chloro-phenyl]-4,5-dihydro-2H-pyridazin-3-one (28) was obtained as off-white solid (1.24 g, 98% yield, >90% pure by LC-MS and ¹H-nmr).

### Synthesis of 6-(4-{3-[(S)-3-(9H-Carbazol-4-yloxy)-2-hydroxy-propylamino]-propoxy}-3-chloro-phenyl)-4,5-dihydro-2H-pyridazin-3-one (29)

To a stirred suspension of 4-oxiranylmethoxy-9H-carbazole (15, 226 mg, 0.95 mmol) in ethanol (80 mL) was added 6-[4-(3-Amino-propoxy)-3-chloro-phenyl]-4,5-dihydro-2H-pyridazin-3-one (28, 400 mg, 1.42 mmol). The mixture was stirred for 3 h under reflux and the solvent was then removed under reduced pressure. The residue was purified by flash column chromatography on silica gel (20 g) eluting with dichloromethane/methanol 100:5 to give 6-(4-{3-[3-(9H-carbazol-4-yloxy)-2-hydroxy-propylamino]-propoxy}-3-chloro-phenyl)-4,5-dihydro-2H-pyridazin-3-one (29) as a light brown powder (340 mg, 65% yield, 97% pure by LC-MS and ¹H-nmr). 2.5 min LC-MS (UV @215 nm: retention time=1.12 min., peak area=100%, TOF-ES⁺ with 25 eV cone voltage: m/z=521 (100%) & 523 (50%)). ¹H NMR: ([D₆]-DMSO, δ in ppm): 11.23 (1H, s), 10.87 (1H, s), 8.20 (1H, d, J=7.78 Hz), 7.75 (1H, d, J=2.20 Hz), 7.60 (1H, dd, J¹=8.69 Hz, J²=2.20 Hz), 7.43 (1H, d, J=8.05 Hz), 7.34-7.23 (2H, m), 7.11 (2H, m), 7.05 (1H, d, J=8.05 Hz), 6.65 (1H, d, J=7.96 Hz), 5.11 (1H, br s), 4.13 (5H, m), 2.88 (3H, m), 2.77 (3H, m), 2.40 (2H, t, J=8.05 Hz), 1.90 (2H, m).

### Example 7

### 6-[4-(2-{4-[(S)-3-(9H-Carbazol-2-yloxy)-2-hydroxy-propylamino]-piperidin-1-yl}-2-oxo-ethoxy)-3-chloro-phenyl]-4,5-dihydro-2H-pyridazin-3-one (33) was synthesized according to Scheme 7.

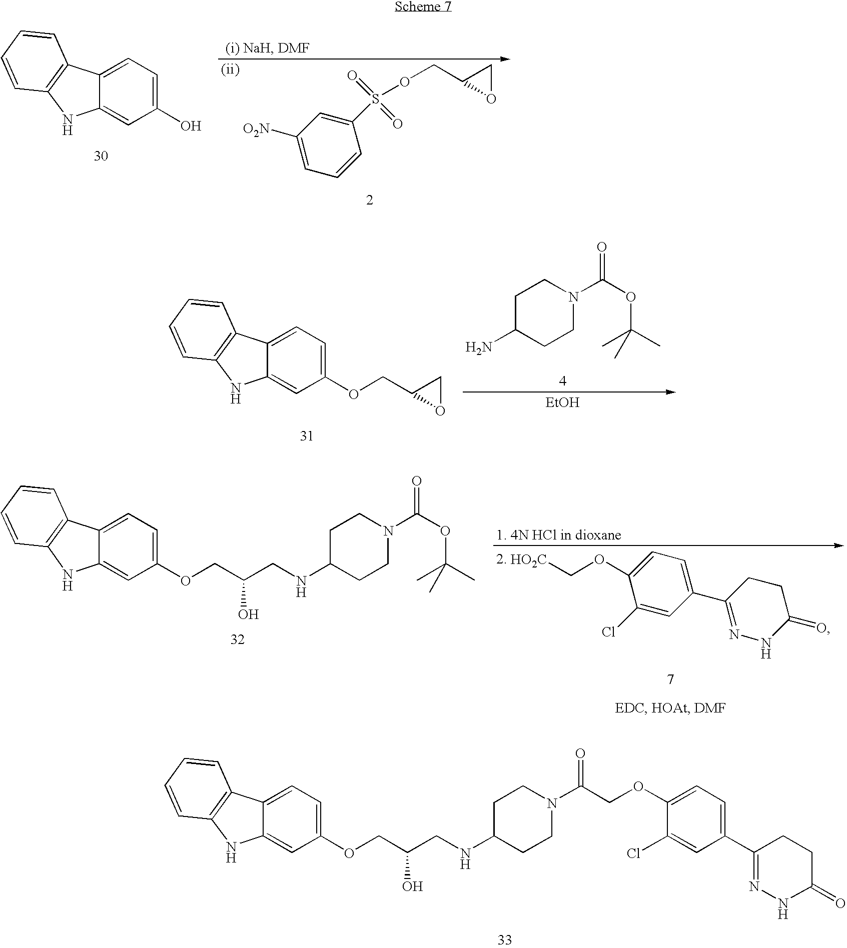

### Synthesis of 6-[4-(2-{4-[(S)-3-(9H-Carbazol-2-yloxy)-2-hydroxy-propylamino]-piperidin-1-yl}-2-oxo-ethoxy)-3-chloro-phenyl]-4,5-dihydro-2H-pyridazin-3-one (33)

Compound 33 was synthesized via the procedure described for Compound 17 using 9H-carbazol-2-ol (30) instead of 9H-carbazol-4-ol (14). A colorless powder (216 mg, 44% yield for final step, 96% pure by LC-MS and ¹H-nmr) was obtained. 2.5 min LC-MS (UV @215 nm: retention time=1.22 min., peak area=96%, TOF-ES⁺ with 25 eV cone voltage: m/z=604.43 (100%) & 606.45 (40%)). ¹H NMR: ([D₆]-DMSO, δ in ppm): 11.32 (1H, s), 11.13 (1H, s), 8.20 (2H, t, J=9.0 Hz), 8.08 (1H, d, J=3.2 Hz), 7.86 (1H, dd, J¹=9.2 Hz, J²=2.8 Hz), 7.65 (1H, d, J=8.4 Hz), 7.54-7.48 (1H, m), 7.36-7.26 (2H, m), 7.20 (1H, d, J=2.0 Hz), 7.01 (1H, dd, J¹=8.4 Hz, J²=2.4 Hz), 5.34-5.19 (3H, m), 4.35 (1H, m), 4.28 (1H, m), 4.23-4.11 (2H, m), 4.01 (1H, br m), 3.40 (1H, d, J=5.2 Hz), 3.38-3.26 (1H, br m), 3.14 (2H, t, J=8.4 Hz), 3.00 (2H, br m), 2.90 (2H, br m), 2.65 (2H, t, J=8.4 Hz), 2.10 (2H, br m), 1.55 (1H, br m), 1.37 (1H, br m).

### Example 8

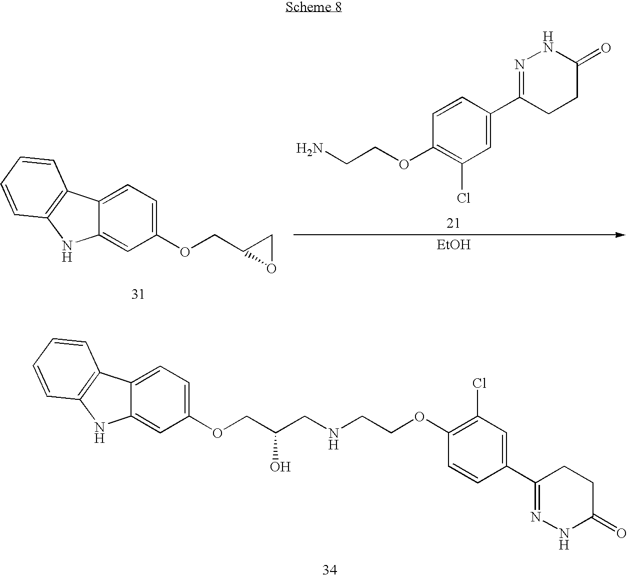

### Synthesis of 6-(4-{2-[3-(9H-carbazol-2-yloxy)-2-hydroxy-propylamino]-ethoxy}-3-chloro-phenyl)-4,5-dihydro-2H-pyridazin-3-one (34)

Compound 34 was synthesized via the procedure described for 22 using 2-((S)-oxiranylmethoxy-9H-carbazole (31) instead of 4-((S)-oxiranylmethoxy-9H-carbazole (15), as shown in Scheme 8. A colorless powder (97 mg, 38% yield for final step, 97% pure by LC-MS and ¹H-nmr) was obtained. 2.5 min LC-MS (UV @215 nm: retention time=1.19 min., peak area=97%, TOF-ES⁺ with 25 eV cone voltage: m/z=507.37 (100%) & 509.39 (40%)). ¹H NMR: ([D₆]-DMSO, δ in ppm): 10.92 (1H, s), 10.74 (1H, s), 7.82 (1H, d, J=7.6 Hz), 7.79 (1H, d, J=8.8 Hz), 7.62 (1H, d, J=2.2 Hz), 7.47 (1H, dd, J¹=9.2 Hz, J²=2.2 Hz), 7.26 (1H, d, J=8.0 Hz), 7.12 (1H, t, J=8.0 Hz), 7.04 (1H, d, J=9.2 Hz), 6.95 (1H, t, J=7.6 Hz), 6.81 (1H, d, J=2.4 Hz), 6.61 (1H, dd, J¹=8.4 Hz, J²=2.0 Hz), 4.92 (1H, d, J=4.8 Hz), 4.02 (2H, t, J=6.0 Hz), 3.91-3.77 (3H, br m), 2.82 (2H, t, J=5.6 Hz), 2.76-2.64 (3H, br m), 2.57 (1H, br m), 2.26 (2H, t, J=9.2 Hz).

### Example 9

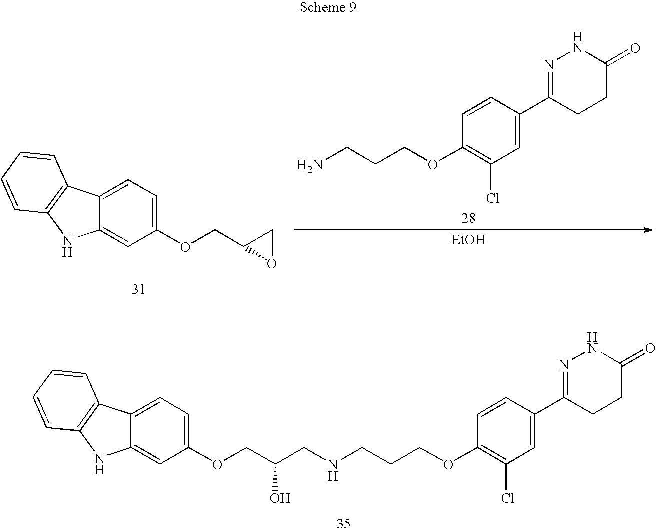

### Synthesis of 6-(4-{3-[(S)-3-(9H-Carbazol-2-yloxy)-2-hydroxy-propylamino]-propoxy)-3-chloro-phenyl)-4,5-dihydro-2H-pyridazin-3-one (35)

Compound 35 was synthesized via the procedure described for 29 using 2-oxiranylmethoxy-9H-carbazole (31) instead of 4-oxiranylmethoxy-9H-carbazole (15) according to Scheme 9. A colorless powder (180 mg, 37% yield for final step, 95% pure by LC-MS and ¹H-nmr) was obtained. 2.5 min LC-MS (UV @215 nm: retention time=1.20 min., peak area=97%, TOF-ES⁺ with 25 eV cone voltage: m/z=521.41 (100%) & 523.37 (40%)). ¹H NMR: ([D₆]-DMSO, δ in ppm): 11.14 (1H, s), 10.95 (1H, s), 8.04 (1H, d, J=8.0 Hz), 8.00 (1H, d, J=8.8 Hz), 7.83 (1H, d, J=2.2 Hz), 7.62 (1H, dd, J¹=8.4 Hz, J²=2.4 Hz), 7.48 (1H, d, J=8.4 Hz), 7.34 (1H, t, J=7.6 Hz), 7.20-7.14 (2H, br m), 7.02 (1H, d, J=2.2 Hz), 6.83 (1H, dd, J¹=8.8 Hz, J²=2.2 Hz), 5.05 (1H, s), 4.22 (2H, t, J=6.4 Hz), 4.10 (1H, m), 4.05-3.97 (2H, br m), 2.91 (2H, t, J=8.4 Hz), 2.80 (3H, br m), 2.71 (1H, br m), 2.46 (2H, t, J=8.8 Hz), 1.97 (2H, m).

### Example 10

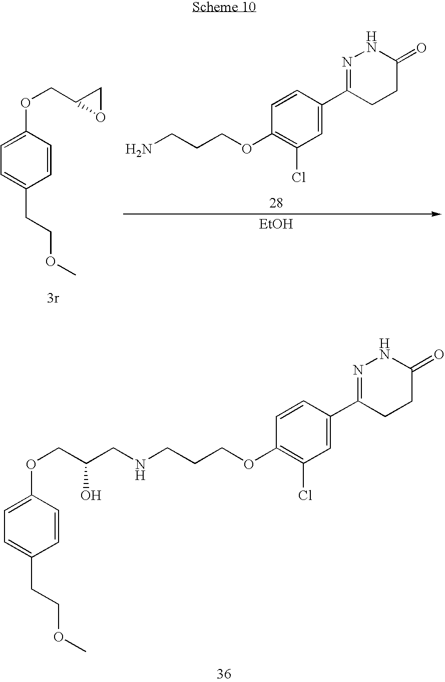

### Synthesis of 6-[3-Chloro-4-(3-{(S)-2-hydroxy-propylamino]-propoxy}-phenyl)-4,5-dihydro-2H-pyridazin-3-one (36)

Compound 36 was synthesized via the procedure described for 29 but using (S)-2-[4-(2-methoxyethyl)-phenoxymethyl]-oxirane (3r) instead of 15 in the last step. A colorless powder (160 mg, 67% yield for the last step), 100% pure by LC-MS and ¹H-nmr was obtained. 2.5 min LC-MS (UV @215 nm: retention time=1.22 min., peak area=100%, TOF-ES⁺ with 25 eV cone voltage: m/z=490.23 (100%) & 492.25 (40%)). ¹H NMR: (CDCl₃, TMS internal standard, δ in ppm): 8.59 (1H, s), 7.77 (1H, d, J=2.4 Hz), 7.56 (1H, dd, J¹=9.2 Hz, J²=2.8 Hz), 7.13 (2H, d, J=8.8 Hz), 6.94 (1H, d, J=8.8 Hz), 6.84 (2H, d, J=8.8 Hz), 4.17 (2H, t, J=6.4 Hz), 4.06 (1H, m), 3.96 (2H, m), 3.56 (2H, t, J=7.2 Hz), 2.97-2.88 (5H, m), 2.85-2.79 (3H, m), 2.60 (2H, m), 2.06 (3H, m).

### Example 11

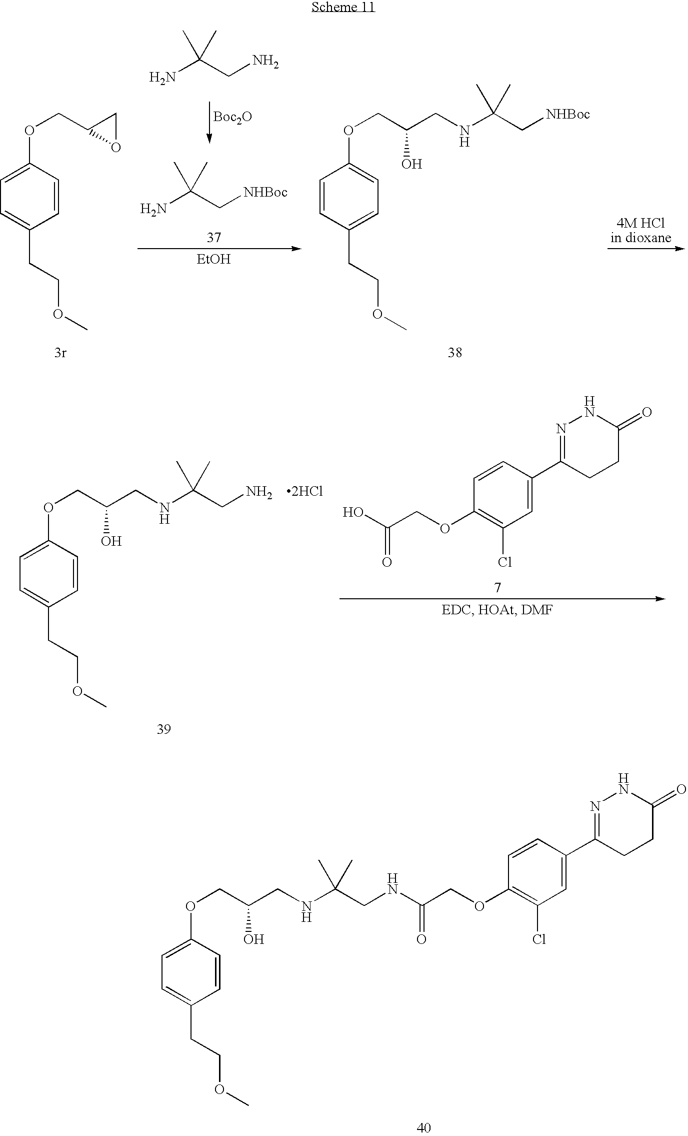

### Synthesis of (2-Amino-2-methyl-propyl)-carbamic acid tert-butyl ester (37)

To a stirred solution of 2-methyl-propane-1,2-diamine (1.00 g, 11.34 mmol) in dichloromethane (15 mL) at −55° C. was added a solution of di-tert-butyl-dicarbonate (2.48 g, 11.36 mmol) in dichloromethane (15 mL) maintaining the temperature below −40° C. The reaction mixture was stirred at −50° C. to −40° C. for 2 h, allowed to warm to ambient temperature over 2.5 h and then stirred at ambient temperature for 1 h. The solution was then extracted with aqueous citric acid solution (10 wt %, 50 mL). The aqueous phase (pH 2-3) was made strongly alkaline (pH 14) with aqueous sodium hydroxide solution (50 wt %, 5 mL) and extracted with dichloromethane (5×25 mL). The combined organic layers were dried (MgSO₄) and concentrated under reduced pressure to give (2-amino-2-methyl-propyl)-carbamic acid tert-butyl ester (37) as a colorless oil which solidified on standing (1.66 g, 78% yield, >95% pure by ¹H-NMR, LC-MS showed no UV response but displayed the expected mass ion with m/z=189).

### Synthesis of 2-[2-Chloro-4-(6-oxo-1,4,5,6-tetrahydro-pyridazin-3-yl)-phenoxy]-N-(2-{(S)-2-hydroxy-3-[4-(2-methoxy-ethyl)-phenoxy]-propylamino]-2-methyl-propyl)-acetamide (40)

The final 3 steps for the synthesis of 40 were carried out in an analogous manner to that described for compounds 8a-r. A colorless powder (150 mg, 67% yield over last 3 steps), 100% pure by LC-MS and ¹H-nmr was obtained. 2.5 min LC-MS (UV @215 nm: retention time=1.17 min., peak area=100%, TOF-ES⁺ with 25 eV cone voltage: m/z=561.30 (100%) & 563.30 (40%)). ¹H NMR: (CDCl₃, TMS internal standard, δ in ppm): 8.60 (1H, s), 7.81 (1H, d, J=2.4 Hz), 7.57 (1H, dd, J¹=8.8 Hz, J²=2.4 Hz), 7.28 (1H, m), 7.08 (2H, d, J=9.2 Hz), 6.89 (1H, d, J=9.2 Hz), 6.81 (2H, d, J=8.8 Hz), 4.54 (2H, s), 3.95 (3H, m), 3.56 (2H, t, J=6.8 Hz), 3.37-3.21 (5H, m), 2.92 (2H, m), 2.80 (3H, m), 2.70 (1H, m), 2.60 (2H, m).

### Example 12

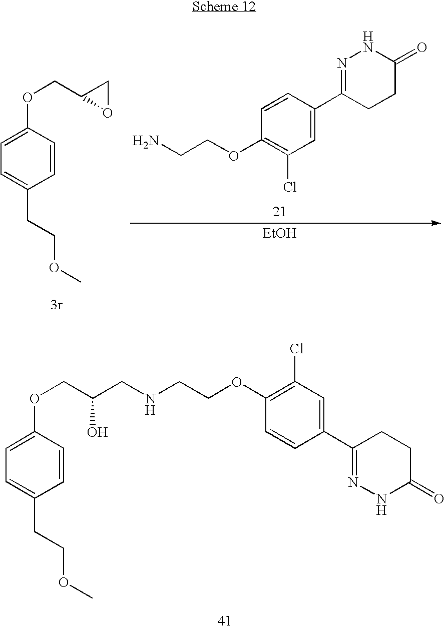

### Synthesis of 6-[3-Chloro-4-(3-{(S)-2-hydroxy-propylamino]-ethoxy}-phenyl)-4,5-dihydro-2H-pyridazin-3-one (41)

Compound 41 was synthesized via the procedure described for 29 but using 2-(4-[2-methoxyethyl]-phenoxymethyl-oxirane (3r) instead of 15 in the last step. A colorless powder (100 mg, 56% yield over last step), 95% pure by LC-MS and ¹H-nmr was obtained. 2.5 min LC-MS (UV @215 nm: retention time=1.67 min., peak area=95%, TOF-ES⁺ with 25 eV cone voltage: m/z=476.26 (100%) & 478.27 (40%)). ¹H NMR: (CDCl₃, TMS internal standard, δ in ppm): 8.73 (1 H, s), 7.76 (1H, d, J=2.4 Hz), 7.55 (1H, dd, J¹=8.4 Hz, J²=2.0 Hz), 6.95 (1H, d, J=9.2 Hz), 6.85 (2H, m), 4.19 (2H, m), 4.11-4.05 (1H, m), 3.99 (2H, d, J=5.2 Hz), 3.56 (2H, t, J=7.2 Hz), 3.14 (2H, t, J=5.2 Hz), 3.02-2.79 (5H, m), 2.60 (2H, m).

### Example 13

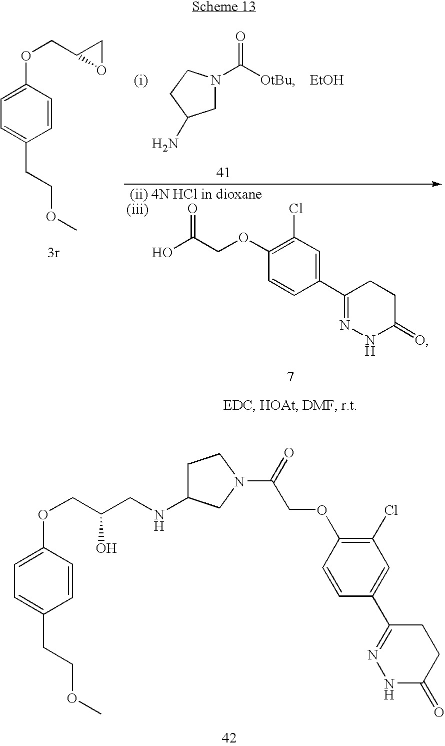

### Synthesis of 6-{3-Chloro-4-[2-(3-{(S)-2-hydroxy-3-[4-(2-methoxy-ethyl)-phenoxy]-propylamino}-pyrrolidin-1-yl)-2-oxo-ethoxy]-phenyl}-4,5-dihydro-2H-pyridazin-3-one (42)

Compound 42 was synthesized via the procedure described for 29 but using (±)-3-amino-1-boc-pyrrolidine (41) instead of 4. A colorless powder (39 mg, 12% yield over last step), 95% pure by LC-MS and ¹H-nmr was obtained. 2.5 min LC-MS (UV @215 nm: retention time=1.65 min., peak area=100%, TOF-ES⁺ with 25 eV cone voltage: m/z=559.29 (100%) & 561.28 (40%)). ¹H NMR: (CDCl₃, TMS internal standard, δ in ppm): 8.52 (1H, s), 7.80 (1H, m), 7.53 (1H, dd, J¹=9.2 Hz, J²=2.5 Hz), 7.14 (1H, d, J=8.8 Hz), 7.00 (1H, m), 6.83 (2H, m), 4.76 (2H, d, J=9.2 Hz), 4.06-3.91 (3H, m), 3.82-3.52 (5H, m), 3.49-3.32 (5H, m), 2.96-2.71 (6H, m), 2.59 (2H, m), 2.20-2.00 (1 H, m), 1.93-1.63 (1H, m).

### Example 14

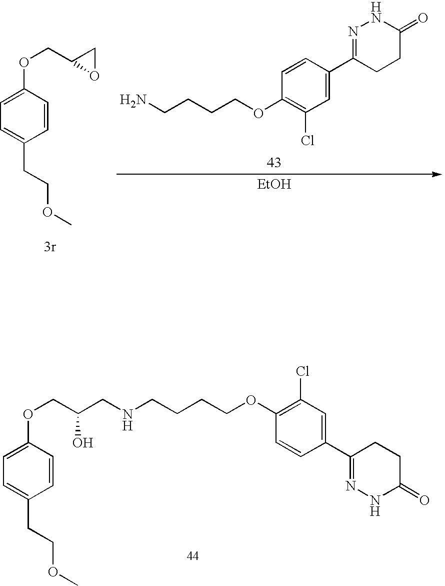

### Synthesis of 6-[3-Chloro-4-(3-{(S)-2-hydroxy-propylamino]-butoxy}-phenyl)-4,5-dihydro-2H-pyridazin-3-one (44)

Compound 44 was synthesized via the procedure described for 41 but using 6-[4-(2-amino-butoxy)-3-chloro-phenyl]-4,5-dihydro-2H-pyridazin-3-one (43) instead of 6-[4-(2-amino-ethoxy)-3-chloro-phenyl]-4,5-dihydro-2H-pyridazin-3-one (21) in the last step. 43 was synthesized in an analogous manner to that described for 21. A colorless powder (99 mg, 49% yield over last step), 100% pure by LC-MS and ¹H-nmr was obtained. 2.5 min LC-MS (UV @215 nm: retention time=1.74 min., peak area=100%, TOF-ES⁺ with 25 eV cone voltage: m/z=504.28 (100%) & 506.30 (40%)). ¹H NMR: (CDCl₃, TMS internal standard, δ in ppm): 8.61 (1H, s), 7.77 (1H, d, J=2.4 Hz), 7.55 (1H, dd, J¹=9.2 Hz, J²=2.8 Hz), 7.13 (2H, m), 6.92 (1H, d, J=9.2 Hz), 6.87-6.82 (2H, m), 4.12-4.03 (3H, m), 3.96 (2H, d, J=5.2 Hz), 3.56 (2H, t, J=7.6 Hz), 3.35 (3H, s), 2.97-2.72 (8H, m), 2.60 (2H, m), 1.93 (2H, m), 1.75 (2H, m).

### Example 15

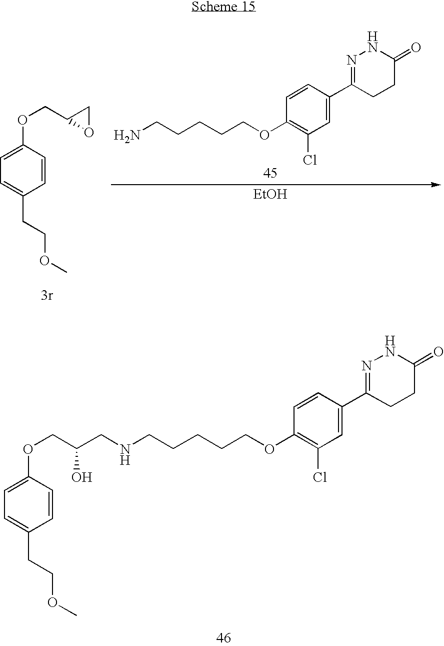

### Synthesis of 6-[3-Chloro-4-(3-{(S)-2-hydroxy-propylamino]-pentoxy}-phenyl)-4,5-dihydro-2H-pyridazin-3-one (46)

Compound 46 was synthesized via the procedure described for 41 but using 6-[4-(2-amino-pentoxy)-3-chloro-phenyl]-4,5-dihydro-2H-pyridazin-3-one (45) instead of 6-[4-(2-amino-ethoxy)-3-chloro-phenyl]-4,5-dihydro-2H-pyridazin-3-one (21) in the last step. 45 was synthesized in an analogous manner to that described for 21. A colorless powder (120 mg, 61% yield over last step), 100% pure by LC-MS and ¹H-nmr was obtained. 2.5 min LC-MS (UV @215 nm: retention time=1.78 min., peak area=100%, TOF-ES⁺ with 25 eV cone voltage: m/z=518.33 (100%) & 520.28 (40%)). ¹H NMR: (CDCl₃, TMS internal standard, δ in ppm): 8.56 (1H, s), 7.77 (1H, d, J=2.4 Hz), 7.55 (1H, dd, J¹=8.8 Hz, J²=2.5 Hz), 7.13 (2H, m), 6.92 (1H, d, J=9.2 Hz), 6.87-6.82 (2H, m), 4.06 (3H, m), 3.96 (2H, d, J=5.2 Hz), 3.56 (2H, t, J=7.6 Hz), 3.35 (3H, s), 2.97-2.65 (8H, m), 2.60 (2H, m), 1.88 (2H, m), 1.59 (4H, m).

### Example 16

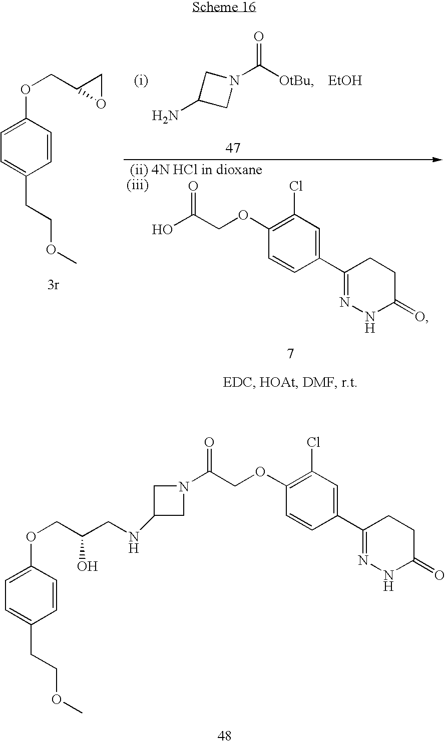

### Synthesis of 6-{3-Chloro-4-[2-(3-{(S)-2-hydroxy-3-[4-(2-methoxy-ethyl)-phenoxy]-propylamino}-azetidin-1-yl)-2-oxo-ethoxy]-phenyl}-4,5-dihydro-2H-pyridazin-3-one (48)

Compound 48 was synthesized via the procedure described for 29 but using 3-amino-1-N-boc-azetidine (47) instead of 4. A colorless powder (40 mg, 15% yield over last step), 95% pure by LC-MS and ¹H-nmr was obtained. 2.5 min LC-MS (UV @215 nm: retention time=1.14 min., peak area=100%, TOF-ES⁺ with 25 eV cone voltage: m/z=545.33 (100%) & 547.31 (40%)). ¹H NMR: (D6-DMSO, δ in ppm): 10.97 (1H, s), 7.86 (1H, d, J=2.0 Hz), 7.71 (1H, dd, J¹=9.2 Hz, J²=2.4 Hz), 7.18 (2H, m), 7.13 (1H, d, J=9.2 Hz), 6.90 (2H, m), 5.08 (1H, m), 4.83 (2H, s), 4.42 (1H, m), 4.09 (1H, m), 4.05-3.92 (2H, m), 3.88 (2H, m), 3.54 (3H, m), 3.28 (3H, s), 2.98 (2H, t, J=8.0 Hz), 2.78 (2H, t, J=11.2 Hz), 2.68 (1H, m), 2.59 (1H, m), 2.49 (2H, t, J=8.8 Hz).

### Example 17

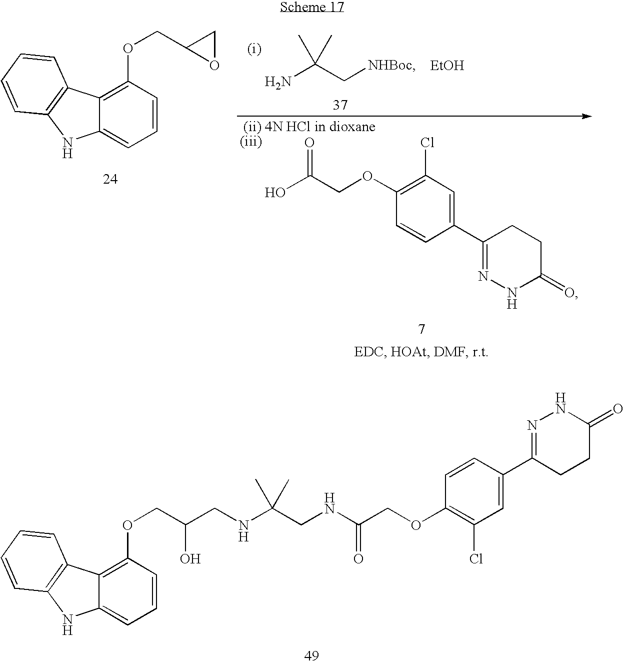

### Synthesis of N-{2-[3-(9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (49)

Compound 49 was synthesized via the procedure described for 40 but using 4-oxiranylmethoxy-9H-carbazole (24) instead of 3r. A colorless powder (49 mg, 24% yield over last step), 100% pure by LC-MS and ¹H-nmr was obtained. 2.5 min LC-MS (UV @215 nm: retention time=1.22 min., peak area=100%, TOF-ES⁺ with 25 eV cone voltage: m/z=592.31 (100%) & 594.32 (40%)). ¹H NMR: (CDCl₃, TMS internal standard, δ in ppm): 8.58 (1H, s), 8.15 (2H, m), 7.62 (1H, d, J=2.4 Hz,), 7.40 (1H, dd, J¹=8.8 Hz, J²=2.8 Hz), 7.31 (2H, m), 7.22 (1H, m), 7.15 (2H, m), 6.94 (1H, d, J=7.6 Hz), 6.71 (1H, d, J=8.8 Hz), 6.54 (2H, d, J=8.4 Hz), 4.42 (2H, s), 4.21-4.06 (3H, m), 3.29 (1H, m), 3.19 (1H, m), 2.84 (2H, m), 2.69 (2H, m), 2.42 (2H, m), 1.08 (6H, s).

### Example 18

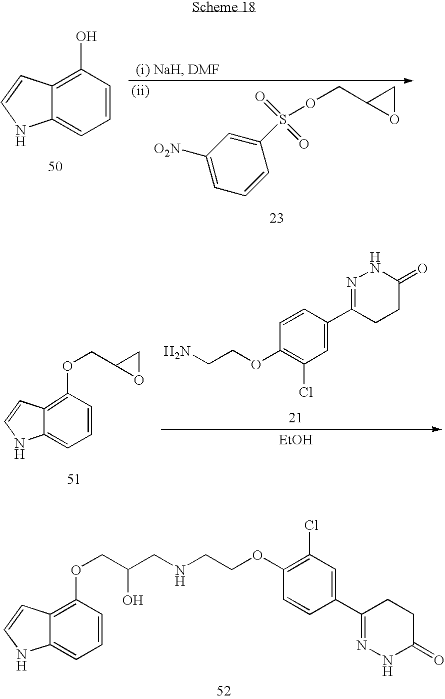

### Synthesis of 6-(3-Chloro-4-{2-hydroxy-3-(1H-indol-4-yloxy)-propylamino]-ethoxy}-phenyl)-4,5-dihydro-2H-pyridazin-3-one (52)

Compound 52 was synthesized via the procedure described for 25 using 4-hydroxyindole (50) instead of 9H-carbazol-4-ol (14). A colorless powder (50 mg, 17% yield over the last two steps, 97% pure by LC-MS and ¹H-nmr) was obtained. 2.5 min LC-MS (UV @215 nm: retention time=1.62 min., peak area=97%, TOF-ES⁺ with 25 eV cone voltage: m/z=457.17 (100%) & 459.20 (40%)). ¹H NMR: ([D₆]-DMSO, δ in ppm): 10.98 (1H, s), 10.83 (1H, s), 7.71 (1H, d, J=2.4 Hz), 7.58 (1H, d, J¹=8.8 Hz, J²=2.4 Hz), 7.13 (2H, m), 6.90 (2H, m), 6.41-6.35 (2H, m), 4.98 (1H, d, J=4.4 Hz), 4.10 (2H, m), 4.00-3.87 (3H, m), 2.91 (1H, t, J=6.0 Hz), 2.88-2.75 (3H, m), 2.67 (1H, m), 2.36 (2H, t, J=8.8 Hz).

### Example 19

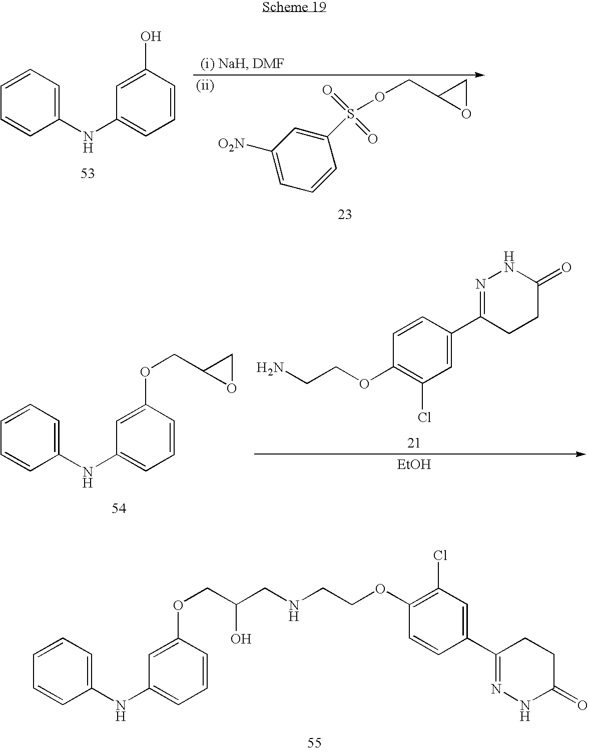

### Synthesis of 6-(3-Chloro-4-{2-hydroxy-3-(3-propylamino-phenoxy)-propylamino]-ethoxy}-phenyl)-4,5-dihydro-2H-pyridazin-3-one (55)

Compound 55 was synthesized via the procedure described for 25 using 3-hydroxydiphenylamine (53) instead of 9H-carbazol-4-ol (14). A colorless powder (50 mg, 16% yield, 95% pure by LC-MS and ¹H-nmr) was obtained. 2.5 min LC-MS (UV @215 nm: retention time=1.83 min., peak area=95%, TOF-ES⁺ with 25 eV cone voltage: m/z=509.20 (100%) & 511.21 (40%)). ¹H NMR: ([D₆]-DMSO, δ in ppm): 11.07 (1H, s), 8.31 (1H, s), 7.95 (1H, d, J=2.4 Hz), 7.83 (1H, d, J¹=9.2 Hz, J²=2.4 Hz), 7.39 (3H, q, J=7.6 Hz), 7.26 (3H, m), 7.00 (1H, t, J=8.0 Hz), 6.79 (1H, m), 6.56 (1H, dd, J¹=8.4 Hz, J²=2.4 Hz), 5.20 (1H, d, J=3.6 Hz), 4.32 (1H, t, J=5.6 Hz), 4.10-3.97 (3H, m), 3.10 (4H, m), 2.94 (1H, m), 2.84 (1H, m), 2.59 (2H, t, J=8.8 Hz).

### Example 20

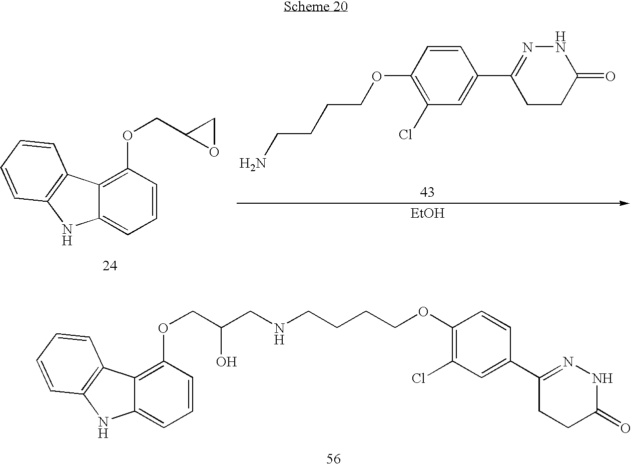

### Synthesis of 6-(4-{3-[3-(9H-Carbazol-4-yloxy)-2-hydroxy-butylamino]-propoxy}-3-chloro-phenyl)-4,5-dihydro-2H-pyridazin-3-one (56)

Compound 56 was synthesized via the procedure described for 25 but using 6-[4-(2-amino-butoxy)-3-chloro-phenyl]-4,5-dihydro-2H-pyridazin-3-one (43) instead of 6-[4-(2-amino-ethoxy)-3-chloro-phenyl]-4,5-dihydro-2H-pyridazin-3-one (21) in the last step. A colorless powder (126 mg, 69% yield for the final step, 95% pure by LC-MS and ¹H-nmr) was obtained. 2.5 min LC-MS (UV @215 nm: retention time=1.80 min., peak area=95%, TOF-ES⁺ with 25 eV cone voltage: m/z=535.28 (100%) & 537.29 (40%)). ¹H NMR: ([D₆]-DMSO, δ in ppm): 11.25 (1H, s), 10.90 (1H, s), 8.22 (1H, d, J=8.0 Hz), 7.77 (1H, d, J=2.4 Hz), 7.63 (1H, dd, J¹=8.8 Hz, J²=2.4 Hz), 7.44 (1H, d, J=8.0 Hz), 7.30 (2H, m), 7.16-7.04 (3H, m), 6.68 (1H, d, J=8.4 Hz), 5.16 (1H, br s), 4.21-4.03 (5H, m), 2.89 (3H, m), 2.78 (1H, m), 2.67 (2H, m), 2.42 (2H, t, J=8.8 Hz), 1.79 (2H, m), 1.61 (2H, m).

### Example 21

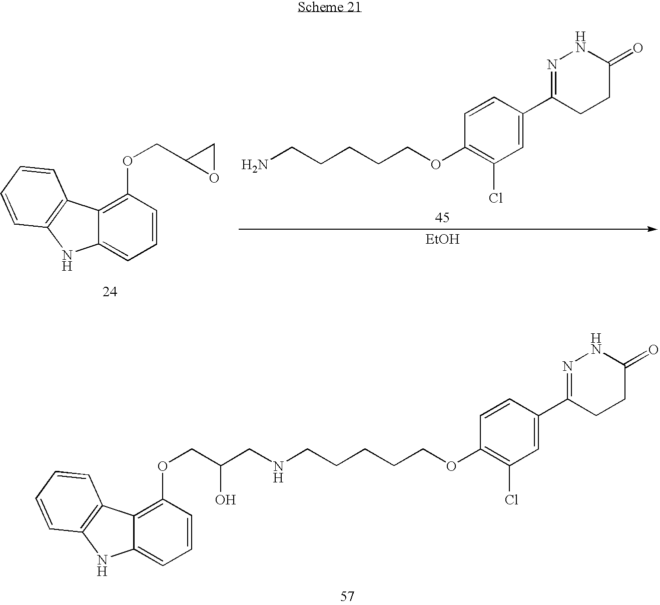

### Synthesis of 6-(4-{3-[3-(9H-Carbazol-4-yloxy)-2-hydroxy-pentylamino]-propoxy}-3-chloro-phenyl)-4,5-dihydro-2H-pyridazin-3-one (57)

Compound 57 was synthesized via the procedure described for 25 but using 6-[4-(2-amino-pentoxy)-3-chloro-phenyl]-4,5-dihydro-2H-pyridazin-3-one (45) instead of 6-[4-(2-amino-ethoxy)-3-chloro-phenyl]-4,5-dihydro-2H-pyridazin-3-one (21) in the last step. A colorless powder (90 mg, 51% yield, 100% pure by LC-MS and ¹H-nmr) was obtained. 2.5 min LC-MS (UV @215 nm: retention time=1.84 min., peak area=100%, TOF-ES⁺ with 25 eV cone voltage: m/z=549.31 (100%) & 551.32 (40%)). ¹H NMR: ([D₆]-DMSO, δ in ppm): 11.25 (1H, s), 10.90 (1H, s), 8.22 (1H, d, J=8.4 Hz), 7.77 (1H, d, J=2.8 Hz), 7.64 (1H, dd, J¹=8.8 Hz, J²=2.4 Hz), 7.44 (1H, d, J=8.0 Hz), 7.30 (2H, m), 7.13 (2H, m), 7.07 (1H, d, J=8.8 Hz), 6.68 (1H, d, J=8.0 Hz), 5.16 (1H, br s), 4.21-4.04 (5H, m), 2.89 (3H, m), 2.78 (1H, m), 2.67 (2H, m), 2.42 (2H, t, J=8.8 Hz), 1.79 (2H, m), 1.66-1.55 (4H, m).

### Example 22

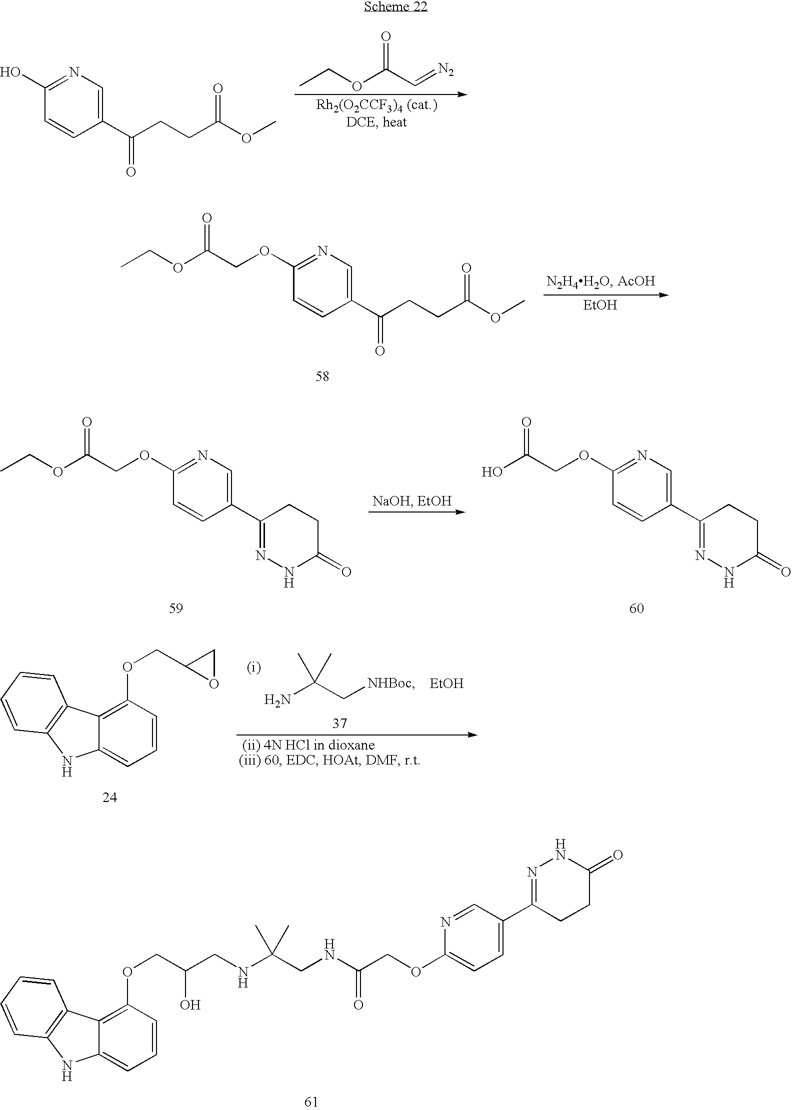

### Synthesis of N-{2-[3-(9H-carbazol-4-yloxy)-2-hydroxy-propylamino]-2-methyl-propyl}-2-[5-(6-oxo-1,4,5,6-tetrahydro-pyridazin-3-yl)-pyridin-2-yloxy]-acetamide (58)

A solution of methyl 4-(6-hydroxy-3-pyridyl)-4-oxo-butyrate (7.73 g, 37 mmol), rhodium (II) trifluoroacetate dimer (0.217 g, 0.33 mmol) in 1,2-dichloroethane (50 ml) was heated to reflux under nitrogen and ethyl diazoacetate (3.77 g, 33 mmol) was added dropwise. The reaction mixture was then stirred at reflux for 4 h before being allowed to cool and dichloromethane (40 ml) was added. The solution was then washed with saturated aqueous sodium bicarbonate solution (50 ml). The aqueous phase was extracted with dichloromethane (3×40 ml) and the combined organic extracts were dried (Na2SO4) and concentrated under reduced pressure to afford the crude product (11.6 g). This product was purified by column chromatography over silica gel (gradient eluent=0-40% ethyl acetate in hexanes) to afford an off-white solid (2.53 g, 23% yield), >90% pure by LCMS.

### Synthesis of [5-(6-Oxo-1,4,5,6-tetrahydro-pyridazin-3-yl)-pyridin-2-yloxy]-acetic acid ethyl ester (59)

To a solution of N-{2-[3-(9H-carbazol-4-yloxy)-2-hydroxy-propylamino]-2-methyl-propyl}-2-[5-(6-oxo-1,4,5,6-tetrahydro-pyridazin-3-yl)-pyridin-2-yloxy]-acetamide (58) (2.53 g, 8.6 mmol) in ethanol (20 ml) was added hydrazine hydrate (0.51 g, 10.3 mmol) and acetic acid (0.62 g, 10.3 mmol). The reaction mixture was stirred at ambient temperature for 18 h before ethyl acetate (20 ml) was added. The resulting precipitate was filtered and the solid was collected and dried with suction to afford an off-white solid (1.72 g, 72% yield), of purity 97% by LCMS.

### Synthesis of [5-(6-Oxo-1,4,5,6-tetrahydro-pyridazin-3-yl)-pyridin-2-yloxy]-acetic acid (60)

To a solution of [5-(6-oxo-1,4,5,6-tetrahydro-pyridazin-3-yl)-pyridin-2-yloxy]-acetic acid ethyl ester (59) (0.70 g, 2.52 mmol) in ethanol-water (1:1) (20 ml) was added a solution of sodium hydroxide (0.505 g, 12.6 mmol) in water (5 ml). The reaction mixture was stirred at ambient temperature for 42 h before 1M HCl (12.6 ml) was added to adjust the solution to pH 6-7, whereupon a precipitate formed. The mixture was then filtered and the collected solid was dried with suction to afford (60) as a white powder (0.612 g, 97% yield), >90% pure by ¹H NMR.

### Synthesis of 6-(4-{3-[3-(9H-Carbazol-4-yloxy)-2-hydroxy-pentylamino]-propoxy}-3-chloro-phenyl)-4,5-dihydro-2H-pyridazin-3-one (61)

Compound 61 was synthesized via the procedure described for 49 but using [5-(6-oxo-1,4,5,6-tetrahydro-pyridazin-3-yl)-pyridin-2-yloxy]-acetic acid (60) instead of [2-chloro-4-(6-oxo-1,4,5,6-tetrahydro-pyridazin-3-yl)-phenoxy]-acetic acid (7) in the last step. A colorless powder (160 mg, 42% yield for the last step, 100% pure by LC-MS and ¹H-nmr) was obtained. 2.5 min LC-MS (UV @215 nm: retention time=1.84 min., peak area=100%, TOF-ES⁺ with 25 eV cone voltage: m/z=549.31 (100%) & 551.32 (40%)). ¹H NMR: ([D₆]-DMSO, δ in ppm): 11.25 (1H, s), 10.90 (1H, s), 8.22 (1H, d, J=8.4 Hz), 7.77 (1H, d, J=2.8 Hz), 7.64 (1H, dd, J¹=8.8 Hz, J²=2.4 Hz), 7.44 (1H, d, J=8.0 Hz), 7.30 (2H, m), 7.13 (2H, m), 7.07 (1H, d, J=8.8 Hz), 6.68 (1H, d, J=8.0 Hz), 5.16 (1H, br s), 4.21-4.04 (5H, m), 2.89 (3H, m), 2.78 (1H, m), 2.67 (2H, m), 2.42 (2H, t, J=8.8 Hz), 1.79 (2H, m), 1.66-1.55 (4H, m).

### Example 23

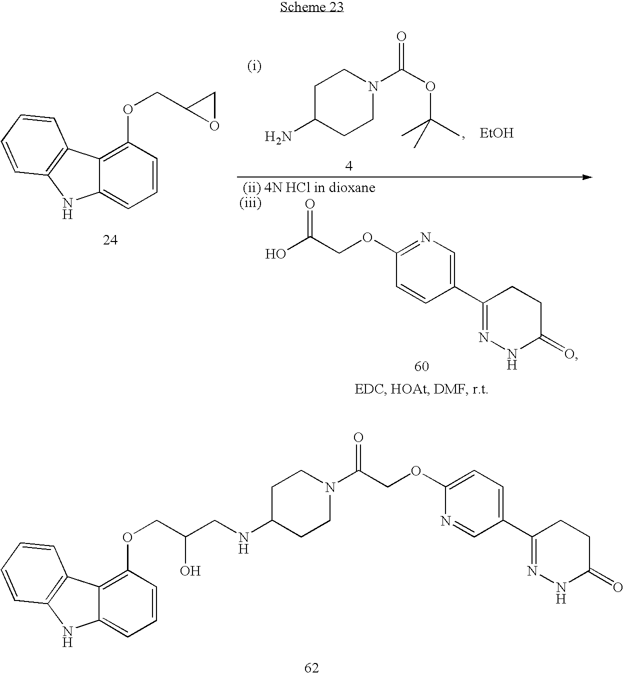

### Synthesis of 6-[6-(2-{4-[3-(9H-Carbazol-4-yloxy)-2-hydroxy-propylamino]-piperidin-1-yl]-4,5-dihydro-2H-pyridazin-3-one (62)

Compound 62 was synthesized via the procedure described for Compound 61 but using 4-amino-piperidine-1-carboxylic acid tert-butyl ester (4) instead of (2-amino-2-methyl-propyl)-carbamic acid tert-butyl ester (37). A colorless powder (100 mg, 24% yield for the last step, 100% pure by LC-MS and ¹H-nmr) was obtained. 2.5 min LC-MS (UV @215 nm: retention time=1.15 min., peak area=100%, TOF-ES⁺ with 25 eV cone voltage: m/z=571.36 (100%). ¹H NMR: ([D₆]-DMSO, δ in ppm): 11.28 (1H, s), 10.95 (1H, s), 8.45 (1H, d, J=2.8 Hz), 8.25 (1H, d, J=9.2 Hz), 8.11 (1H, dd, J¹=8.8 Hz, J²=2.4 Hz), 7.48 (1H, d, J=8.8 Hz), 7.35 (2H, m), 7.17 (1H, t, J=8.0 Hz), 7.10 (1H, d, J=8.4 Hz), 6.95 (1H, d, J=8.8 Hz), 6.72 (1H, d, J=8.4 Hz), 5.22-5.09 (3H, m), 4.26-4.04 (4H, m), 3.77 (1H, m), 3.11 (1H, t, J=12.4 Hz), 2.95 (3H, t, J=8.4 Hz), 2.86-2.69 (3H, m), 2.46 (2H, t, J=8.8 Hz), 1.98-1.77 (2H, br m), 1.33 (1H, br m), 1.16 (1H, br m).

### Example 24

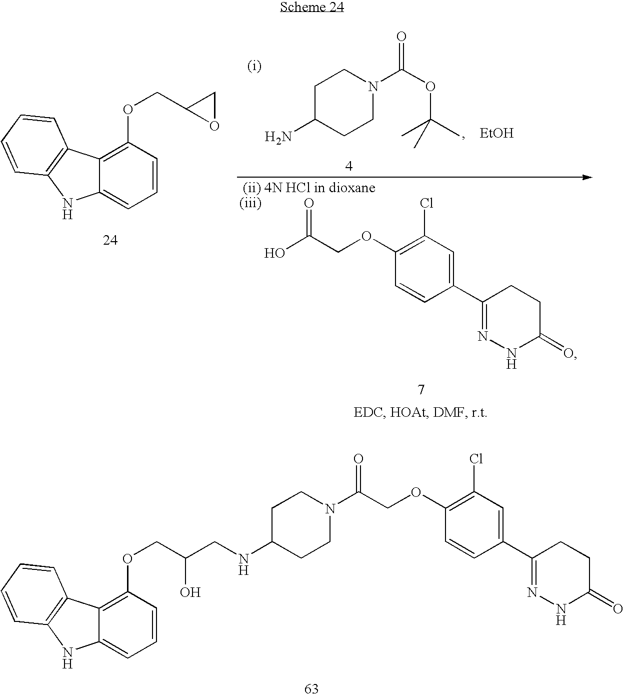

### Synthesis of 6-[4-(2-{4-[3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (63)

Compound 63 was synthesized via the procedure described for 62 but using [2-chloro-4-(6-oxo-1,4,5,6-tetrahydro-pyridazin-3-yl)-phenoxy]-acetic acid (7) instead of [5-(6-oxo-1,4,5,6-tetrahydro-pyridazin-3-yl)-pyridin-2-yloxy]-acetic acid (60) in the last step, as shown in Scheme 24. A colorless powder (260 mg, 49% yield for the last step, 100% pure by LC-MS and ¹H-nmr) was obtained. 2.5 min LC-MS (UV @215 nm: retention time=1.15 min., peak area=100%, TOF-ES⁺ with 25 eV cone voltage: m/z=604.02 (100%) & 606.01 (40%). ¹H NMR: ([D₆]-DMSO, δ in ppm): 11.31 (1H, s), 10.96 (1H, s), 8.27 (1H, d, J=8.4 Hz), 7.84 (1H, d, J=2.4 Hz), 7.68 (1H, dd, J¹=8.8 Hz, J²=2.4 Hz), 7.51 (1H, d, J=8.4 Hz), 7.38 (2H, m), 7.20 (1H, t, J=8.0 Hz), 7.15-7.08 (2H, m), 6.75 (1H, d, J=8.0 Hz), 5.20 (1H, br s), 5.07 (2H, m), 4.27-4.09 (4H, m), 3.82 (1H, br m), 3.15 (1H, m), 2.97 (3H, t, J=8.4 Hz), 2.91-2.72 (3H, m), 2.48 (2H, t, J=8.8 Hz), 1.90 (2H, br m), 1.37 (1H, br m), 1.19 (1H, br m).

### Example 25

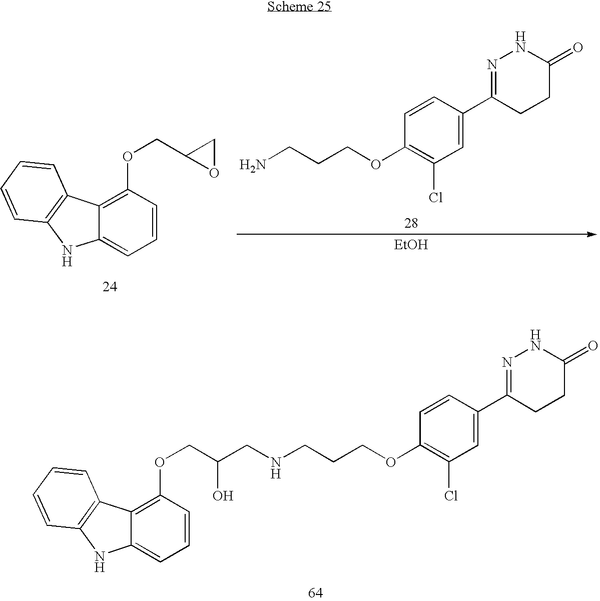

### Synthesis of 6-(4-{3-[3-(9H-Carbazol-4-yloxy)-2-hydroxy-propylamino]-propoxy}-3-chloro-phenyl)-4,5-dihydro-2H-pyridazin-3-one (64)

Compound 64 was synthesized via the procedure described for 29 using 4-oxiranylmethoxy-9H-carbazole (24) instead of 4-((S)-4-oxiranylmethoxy-9H-carbazole (15). A colorless powder (200 mg, 46% yield for the last step, 100% pure by LC-MS and ¹H-nmr) was obtained. 2.5 min LC-MS (UV @215 nm: retention time=1.75 min., peak area=100%, TOF-ES⁺ with 25 eV cone voltage: m/z=521.23 (100%) & 523.23 (40%). ¹H NMR: ([D₆]-DMSO, δ in ppm): 11.17 (1H, s), 10.82 (1H, s), 8.14 (1H, d, J=8.8 Hz), 7.69 (1H, d, J=2.4 Hz), 7.54 (1H, dd, J¹=8.8 Hz, J²=2.4 Hz), 7.37 (1H, d, J=8.4 Hz), 7.25 (1H, m), 7.20 (1H, t, J=8.4 Hz), 7.08-6.97 (3H, m), 6.59 (1H, d, J=8.4 Hz), 5.04 (1H, br s), 4.14-4.00 (5H, m), 2.81 (3H, m), 2.70 (3H, m), 2.34 (2H, t, J=8.8 Hz).

### Example 26

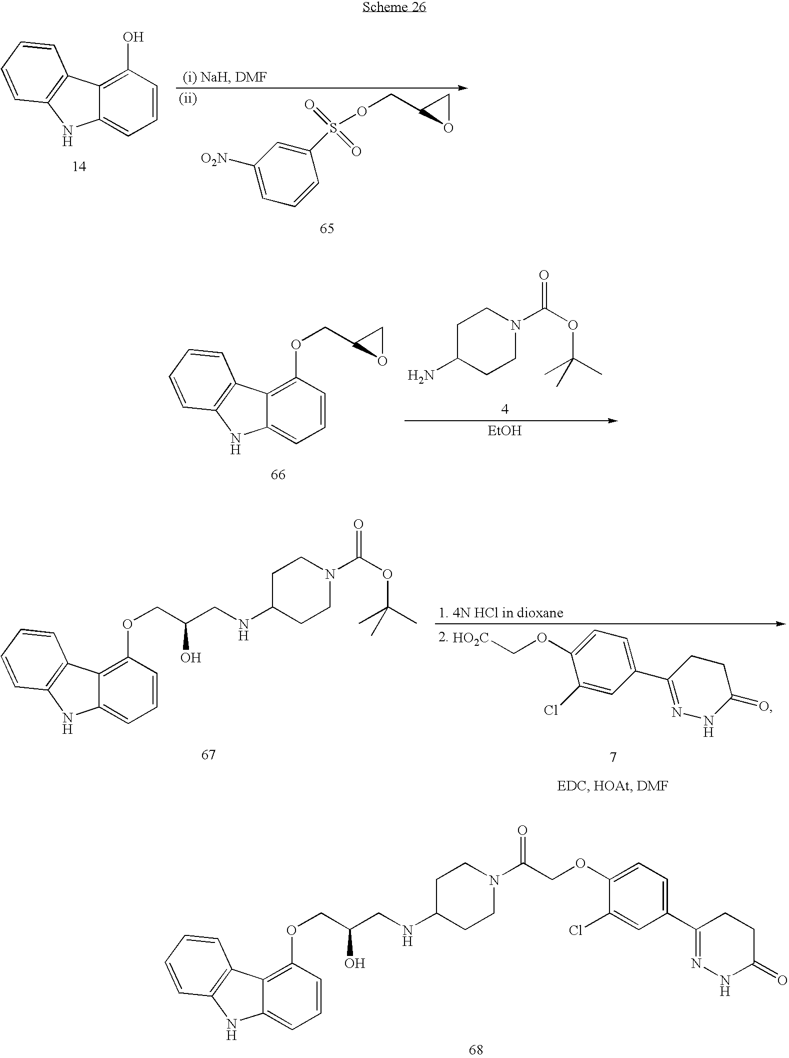

### Synthesis of 6-[4-(2-{4-[3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (68)

Compound 68 was synthesized via the procedure described for 17 but using (2R)-glycidyl m-nitrobenzenesulfonate (65) instead of (2S)-glycidyl m-nitrobenzenesulfonate (2) in the first step. A colorless powder (330 mg, 53% yield for the last step, 97% pure by LC-MS and ¹H-nmr) was obtained. 2.5 min LC-MS (UV @215 nm: retention time=1.75 min., peak area=97%, TOF-ES⁺ with 25 eV cone voltage: m/z=521.22 (100%) & 523.18 (40%). ¹H NMR: ([D₆]-DMSO, δ in ppm): 11.00 (1H, s), 10.65 (1H, s), 7.97 (1H, d, J=8.4 Hz), 7.52 (1H, d, J=2.0 Hz), 7.37 (1H, dd, J¹=8.8 Hz, J²=2.0 Hz), 7.20 (1H, d, J=8.4 Hz), 7.08 (1H, t, J=8.0 Hz), 7.04 (1H, t, J=8.0 Hz), 6.91-6.80 (3H, m), 6.42 (1H, d, J=8.0 Hz), 4.87 (1H, br s), 3.97-3.81 (5H, m), 2.65 (3H, m), 2.53 (3H, m), 2.18 (2H, t, J=8.8 Hz), 1.66 (2H, m).

### Example 27

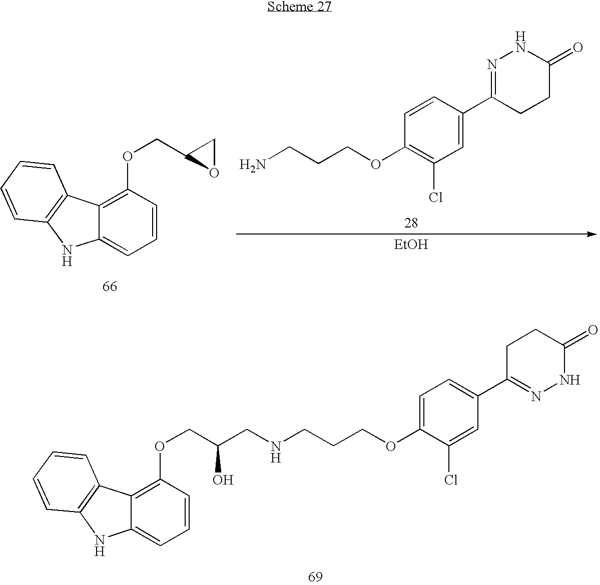

### Synthesis of 6-(4-{3-[3-(9H-Carbazol-4-yloxy)-2-hydroxy-propylamino]-propoxy}-3-chloro-phenyl)-4,5-dihydro-2H-pyridazin-3-one (69)

Compound 69 was synthesized via the procedure described for 29 using 4-((R)-4-oxiranylmethoxy-9H-carbazole (66) instead of 4-((S)-4-oxiranylmethoxy-9H-carbazole (15). A colorless powder (270 mg, 62% yield for the last step, 100% pure by LC-MS and ¹H-nmr) was obtained. 2.5 min LC-MS (UV @215 nm: retention time=1.75 min., peak area=100%, TOF-ES⁺ with 25 eV cone voltage: m/z=521.23 (100%) & 523.23 (40%). ¹H NMR: ([D₆]-DMSO, δ in ppm): 11.17 (1H, s), 10.82 (1H, s), 8.14 (1H, d, J=8.8 Hz), 7.69 (1H, d, J=2.4 Hz), 7.54 (1H, dd, J¹=8.8 Hz, J²=2.4 Hz), 7.37 (1H, d, J=8.4 Hz), 7.25 (1H, m), 7.20 (1H, t, J=8.4 Hz), 7.08-6.97 (3H, m), 6.59 (1H, d, J=8.4 Hz), 5.04 (1H, br s), 4.14-4.00 (5H, m), 2.81 (3H, m), 2.70 (3H, m), 2.34 (2H, t, J=8.8 Hz).

### Example 28

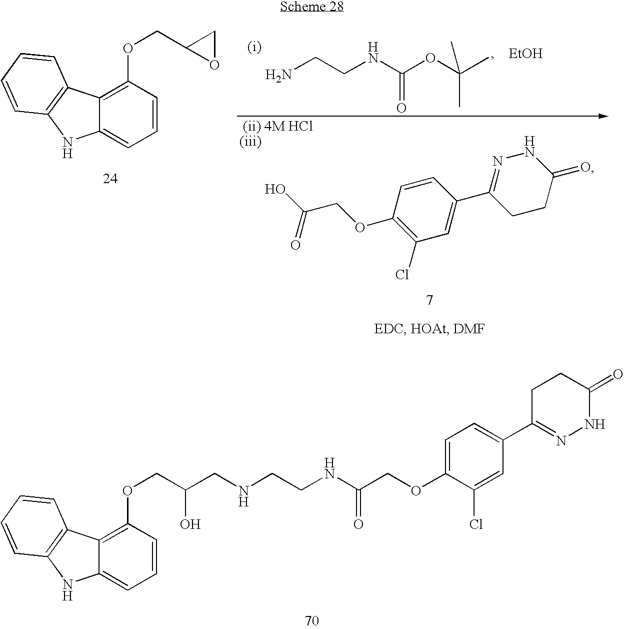

### Synthesis of 6-(4-{3-[3-(9H-Carbazol-4-yloxy)-2-hydroxy-propylamino]-propoxy}-3-chloro-phenyl)-4,5-dihydro-2H-pyridazin-3-one (70)

Compound 70 was synthesized via the procedure described for 49 using tert-butyl N-(2-aminoethyl)carbamate instead of (2-amino-2-methyl-propyl)-carbamic acid tert-butyl ester (37). A colorless powder (80 mg, 39% yield for the last step, 98% pure by LC-MS and ¹H-nmr) was obtained. 2.5 min LC-MS (UV @215 nm: retention time=1.23 min., peak area=98%, TOF-ES⁺ with 25 eV cone voltage: m/z=564.18 (100%) & 566.17 (40%). ¹H NMR: ([D₆]-DMSO, δ in ppm): 11.30 (1H, s), 10.97 (1H, s), 8.27 (1H, d, J=8.4 Hz), 8.04 (1H, t, J=6.4 Hz), 7.85 (1H, d, J=2.4 Hz), 7.69 (1H, dd, J¹=8.4 Hz, J²=2.4 Hz), 7.50 (1H, d, J=8.4 Hz), 7.36 (2H, m), 7.19 (1H, t, J=8.0 Hz), 7.33 (1H, t, J=8.0 Hz), 7.22-7.09 (3H, m), 6.73 (1H, d, J=8.6 Hz), 5.18 (1H, br s), 4.70 (1H, s), 4.27-4.10 (3H, m), 3.32 (2H, m), 2.92 (3H, m), 2.82 (1H, J¹=12.0 Hz, J²=6.9 Hz), 2.75 (2H, t, J=6.3 Hz).

### Example 29

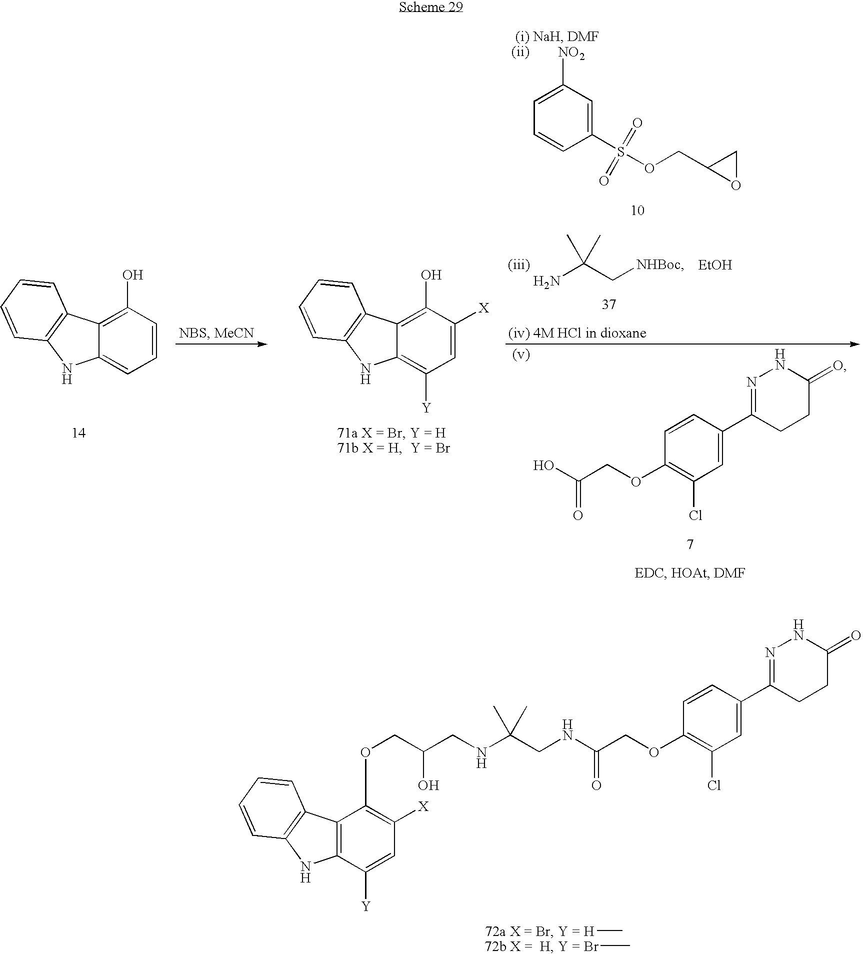

### Synthesis of 3-bromo-9H-carbazol-4-ol (71a)

To a solution of 9H-carbazol-4-ol (14) (0.80 g, 4.37 mmol) in acetonitrile (10 ml) was added N-bromosuccinimide (0.727 g, 4.37 mmol) and the reaction mixture was stirred at ambient temperature for 30 minutes before being concentrated under reduced pressure. The residue was then purified by flash column chromatography over silica gel (gradient eluent=85% heptanes in dichloromethane to 75% heptanes in dichloromethane). 71a (0.40 g, 34% yield) was isolated as a grey solid, 96% pure by LCMS.

### Synthesis of 1-bromo-9H-carbazol-4-ol (71b)

This was synthesized using the same procedure for 71a. 71b (0.214 g, 19% yield) was isolated as a grey solid, 100% pure by LCMS.

### Synthesis of N-{2-[3-(3-Bromo-9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (72a)

Compound 72a was synthesized in an analogous fashion to that described for 8a except 3-bromo-9H-carbazol-4-ol (71a) was used in the instead of phenol (1a). A colorless powder (160 mg, 91% yield over last step), 95% pure by LC-MS and ¹H-nmr was obtained. 2.5 min LC-MS (UV @215 nm: retention time=1.78 min., peak area=95%, TOF-ES⁺ with 25 eV cone voltage: m/z=670.25 (70%) & 672.25 (100%)). ¹H NMR: ([D₆]-DMSO, δ in ppm): 11.51 (1H, s), 10.90 (1H, s), 8.40 (1H, d, J=8.4 Hz), 7.74 (1H, d, J=2.0 Hz,), 7.66 (1H, t, J=6.0 Hz), 7.61 (1H, dd, J¹=8.6 Hz, J²=2.4 Hz), 7.51 (2H, m), 7.42 (1H, m), 7.20 (2H, m), 7.08 (1H, d, J=9.2 Hz), 5.22 (1H, d, J=4.8 Hz), 4.67 (1H, s), 4.15-4.02 (3H, m), 3.12 (2H, d, J=6.0 Hz), 2.86 (1H, t, J=8.4 Hz), 2.76 (1H, m), 2.68 (1H, m), 2.39 (2H, t, J=8.8 Hz), 1.63 (1H, br s), 1.01 (6H, d, J=4.4 Hz).

### Synthesis of N-{2-[3-(1-Bromo-9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (72b)

Compound 72b was synthesized in an analogous fashion to that described for 8a except 1-bromo-9H-carbazol-4-ol (71b) was used in the instead of phenol (1a). A colorless powder (131 mg, 82% yield over last step), 95% pure by LC-MS and ¹H-nmr was obtained. 2.5 min LC-MS (UV @215 nm: retention time=1.83 min., peak area=95%, TOF-ES⁺ with 25 eV cone voltage: m/z=670.28 (70%) & 672.25 (100%)). ¹H NMR: ([D₆]-DMSO, δ in ppm): 11.36 (1H, s), 10.90 (1H, s), 8.21 (1H, d, J=8.0 Hz), 7.73 (1H, d, J=2.0 Hz,), 7.62 (2H, m), 7.54 (1H, d, J=8.4 Hz), 7.38 (2H, m), 7.16 (1H, t, J=8.0 Hz), 7.04 (1H, d, J=8.8 Hz), 6.62 (1H, d, J=8.8 Hz), 5.12 (1H, d, J=5.2 Hz), 4.57 (2H, s), 4.25-4.09 (2H, m), 4.00 (1H, m), 3.11 (2H, d, J=5.6 Hz), 2.86 (1H, t, J=8.4 Hz), 2.82-2.66 (2H, m), 2.40 (2H, t, J=8.4 Hz), 1.69 (1H, br s), 1.01 (6H, s).

### Example 30

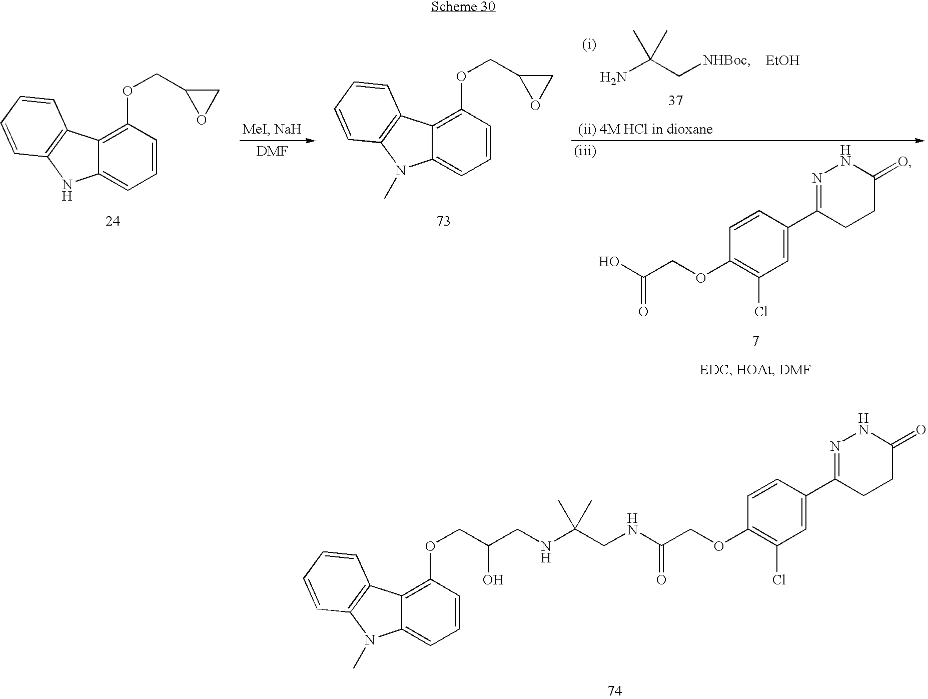

### Synthesis of 9-Methyl-4-oxiranylmethoxy-9H-carbazole (73)

To a stirred suspension of sodium hydride (60% dispersion in mineral oil, 67 mg, 1.67 mmol) in N,N-dimethylformamide (4 mL) under N₂ at 0° C. was added portionwise a solution of 4-oxiranylmethoxy-9H-carbazole (24) (200 mg, 0.836 mmol) in N,N-dimethylformamide (4 mL) followed by methyl iodide (55 μl, 0.92 mmol) and the reaction mixture was stirred at ambient temperature for 4 h. Following this, ethyl acetate (50 ml) and water (50 ml) were added and the two-phase system was shaken and separated. The aqueous phase was then extracted with ethyl acetate (2×25 ml) and the combined organic extracts were washed with water (25 ml) and saturated brine (2×25 ml) then dried (Na₂SO₄) and concentrated under reduced pressure to afford a pale brown solid (205 mg, 97% yield) of purity 98% by LCMS.

### Synthesis of 2-(2-Chloro-4-(6-oxo-1,4,5,6-tetrahydro-pyridazin-3-yl)-phenoxy]-N-{2-[2-hydroxy-3-(9-methyl-9H-carbazol-yloxy)-propylamino]-2-methyl-propyl}-2-methyl-propyl}-acetamide (74)

Compound 74 was synthesized in an analogous fashion to that described for 8a except 9-methyl-4-oxiranylmethoxy-9H-carbazole (73) was used in the instead of phenol (1a). A colorless powder (120 mg, 54% yield over last step), 100% pure by LC-MS and ¹H-nmr was obtained. 2.5 min LC-MS (UV @215 nm: retention time=1.83 min., peak area=100%, TOF-ES⁺ with 25 eV cone voltage: m/z=606.32 (100%) & 608.32 (40%)). ¹H NMR: ([D₆]-DMSO, δ in ppm): 10.90 (1H, s), 8.24 (1H, d, J=8.4 Hz), 7.74 (1H, d, J=2.2 Hz,), 7.62 (2H, m), 7.55 (1H, d, J=8.4 Hz), 7.41 (1H, t, J=8.2 Hz), 7.30 (1H, t, J=8.4 Hz), 7.14 (1H, m), 7.05 (1H, d, J=9.2 Hz), 5.10 (1H, d, J=5.2 Hz), 4.60 (2H, s), 4.18 (2H, m), 4.02 (1H, m), 3.83 (3H, s), 3.11 (1H, t, J=5.6 Hz), 2.90-2.77 (3H, m), 2.71 (1H, m), 2.39 (2H, t, J=8.8 Hz), 1.69 (1H, br s), 1.01 (6H, s).

### Example 31

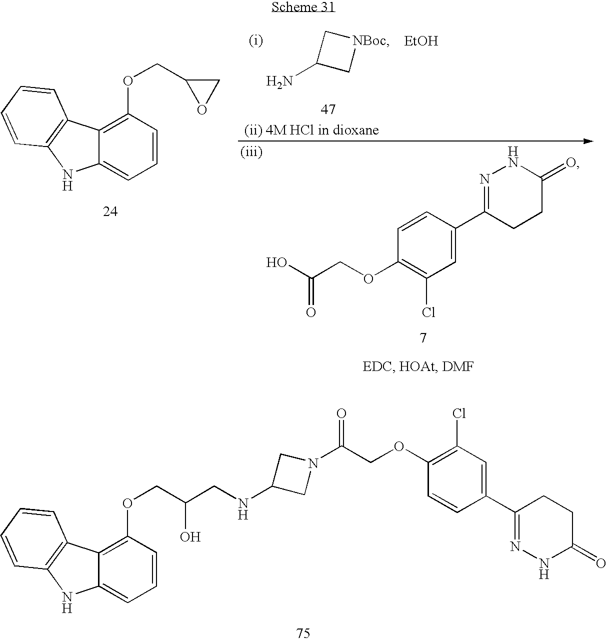

### Synthesis of 6-[4-(2-{3-[3-(9H-Carbazol-4-yloxy)-2-hydroxy-propylamino]-azetidin-1-yl}-2-oxo-ethoxy)-3-chloro-phenyl]-4,5-dihydro-2H-pyridazin-3-one (75)

Compound 75 was synthesized via the procedure described for 48 but using 4-oxiranylmethoxy-9H-carbazole (24) instead of 3r. A colorless powder (90 mg, 26% yield over last step), 100% pure by LC-MS and ¹H-nmr was obtained. 2.5 min LC-MS (UV @215 nm: retention time=1.70 min., peak area=100%, TOF-ES⁺ with 25 eV cone voltage: m/z=576.40 (100%) & 578.40 (40%)). ¹H NMR: ([D₆]-DMSO, δ in ppm): 11.22 (1H, s), 10.88 (1H, s), 8.18 (1H, d, J=8.4 Hz), 7.76 (1H, d, J=2.2 Hz,), 7.61 (1H, dd, J¹=8.8 Hz, J²=2.4 Hz), 7.42 (1H, d, J=8.2 Hz), 7.28 (2H, m), 7.11 (1H, t, J=8.4 Hz), 7.03 (2H, m), 6.65 (1H, d, J=8.0 Hz), 5.15 (1H, br s), 4.72 (2H, s), 4.35 (1H, t, J=8.0 Hz), 4.17-3.92 (5H, m), 3.64 (2H, m), 2.88 (1H, t, J=8.4 Hz), 2.88 (2H, t, J=8.4 Hz), 2.80 (1H, m), 2.70 (2H, m), 2.40 (2H, t, J=8.8 Hz).

### Example 32

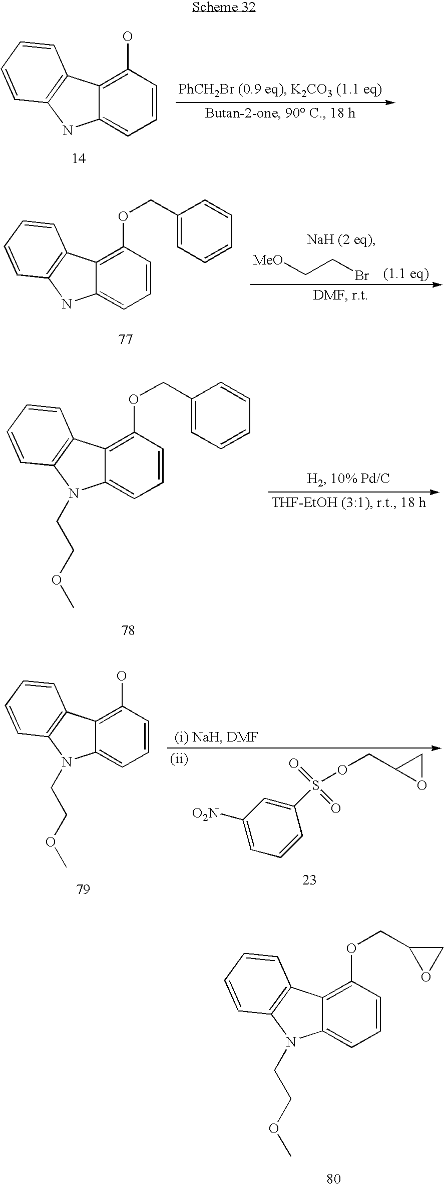

### Synthesis of 4-benzyloxy-9H-carbazole (77)

To a stirred solution of 9H-carbazol-4-ol (14, 3.0 g, 16.4 mmol) and benzyl bromide (2.52 g, 14.7 mmol) in butan-2-one (150 mL) was added potassium carbonate (2.49 g, 18.0 mmol). The mixture was stirred under reflux for 18 h and the solvent was then removed under reduced pressure. The residue was re-dissolved in dichloromethane (150 mL) and the solution was washed with aqueous sodium hydroxide (1N, 2×50 mL), water (50 mL) and saturated brine (50 mL). The organic layer was dried over sodium sulphate and evaporated to dryness. 4-Benzyloxy-9H-carbazole (77) was isolated as a brown solid (4.2 g, 88% yield, 91% pure by LC-MS and ¹H-nmr).

### Synthesis of 4-benzyloxy-9-(2-methoxy-ethyl)-9H-carbazole (78)

To a stirred solution of 4-benzyloxy-9H-carbazole (77, 1.0 g, 3.66 mmol) in N,N-dimethylformamide (20 mL) was added sodium hydride (293 mg, 7.32 mmol) and the reaction mixture was stirred at ambient temperature for 30 min. Following this, 2-bromoethyl methyl ether (564 mg, 4.03 mmol) diluted in N,N-dimethylformamide (12 mL) was added and the mixture was stirred at ambient temperature for 18 h. The reaction mixture was quenched with water (80 mL) and extracted with Ethyl Acetate (3×80 mL) and the combined organic layers were washed with saturated brine (4×100 mL). The organic layers were dried over sodium sulphate and evaporated to dryness. The crude mixture was purified by flash column chromatography over Silica gel (30 g) eluting with a gradient of hexane/dichloromethane (9:1) to neat dichloromethane. Fractions with R_{f}=0.50 (dichloromethane) were combined and concentrated under reduced pressure to give 4-Benzyloxy-9-(2-methoxy-ethyl)-9H-carbazole (78) as a colorless gum (1.00 g, 78% yield, 95% pure by LC-MS and ¹H-nmr).

### Synthesis of 9-(2-methoxy-ethyl)-9H-carbazol-4-ol (79)

To a stirred solution of 4-benzyloxy-9-(2-methoxy-ethyl)-9H-carbazole (78, 500 mg, 1.51 mmol) in tetrahydrofuran-ethanol (3 to 1 mixture by volume) (20 mL) was added 10% Pd on carbon (161 mg, 0.15 mmol) and the reaction mixture was stirred at ambient temperature for 18 h. Following this, the suspension was filtered through a pad of celite and washed with ethyl acetate (100 mL). The filtrate was then taken and concentrated under reduced pressure to afford 9-(2-methoxy-ethyl)-9H-carbazol-4-ol (79) as a brown gum (394 mg, 75% yield, 83% pure by LC-MS and ¹H-nmr).

### Synthesis of 9-(2-methoxy-ethyl)-4-oxiranylmethoxy-9H-carbazole (80)

9-(2-Methoxy-ethyl)-4-oxiranylmethoxy-9H-carbazole (80) was synthesized using the procedure described for the synthesis of (24). A brown powder (425 mg, 85% yield, 91% pure by LC-MS and ¹H-nmr) was obtained.

### Example 33

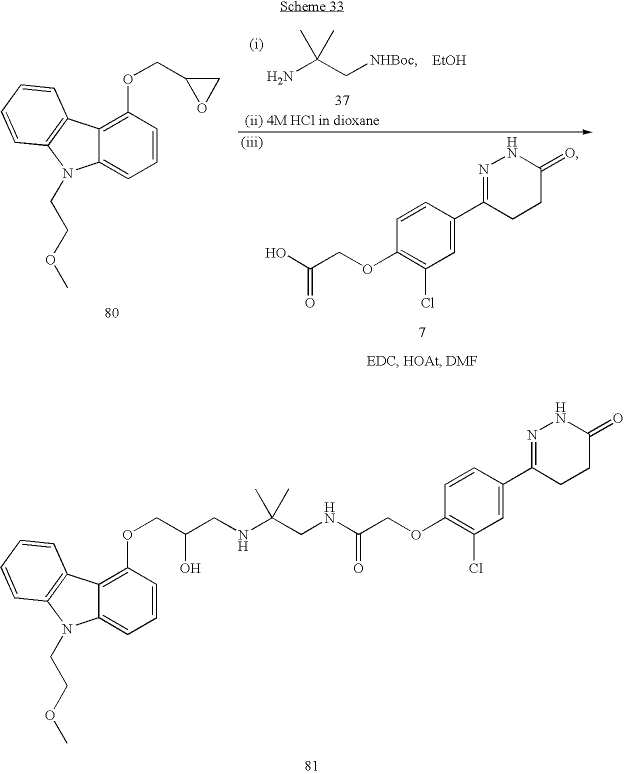

### Synthesis of 2-[2-chloro-4-(6-oxo-1,4,5,6-tetrahydro-pyridazin-3-yl)-phenoxy]-N-(2-{2-hydroxy-3-[9-(2-methoxy-ethyl)-9H-carbazol-4-yloxy]-propylamino}-2-methyl-propyl)-acetamide (81)

Compound 81 was synthesized via the procedure described for 49 but using 9-(2-methoxy-ethyl)-4-oxiranylmethoxy-9H-carbazole (80) instead of (24). A white solid (222 mg, 24% yield over 3 steps), 100% pure by LC-MS and ¹H-nmr was obtained. 2.5 min LC-MS (UV @215 nm: retention time=1.64 min., peak area=100%, TOF-ES⁺ with 25 eV cone voltage: m/z=650.58 (100%) & 652.55 (40%)). ¹H NMR: ([D₆]-DMSO, δ in ppm): 10.90 (1H, s), 8.23 (1H, d, J=8.0 Hz), 7.76 (1H, d, J=2.4 Hz), 7.68-7.59 (2H, m), 7.38 (1H, m), 7.28 (1H, t, J=8.4 Hz), 7.18-7.10 (2H, m), 7.07 (1H, d, J=9.2 Hz), 6.69 (1H, d, J=8.4 Hz), 5.09 (1H, d, J=5.2 Hz), 4.62 (2H, m), 4.51 (2H, t, J=5.6 Hz), 4.17 (2H, m), 4.02 (1H, m), 3.69 (2H, t, J=5.6 Hz), 3.17 (3H, s), 3.11 (2H, m), 2.85 (2H, t, J=8.8 Hz), 2.71 (2H, m), 2.38 (2H, t, J=9.2 Hz), 1.02 (6H, s).

### Example 34

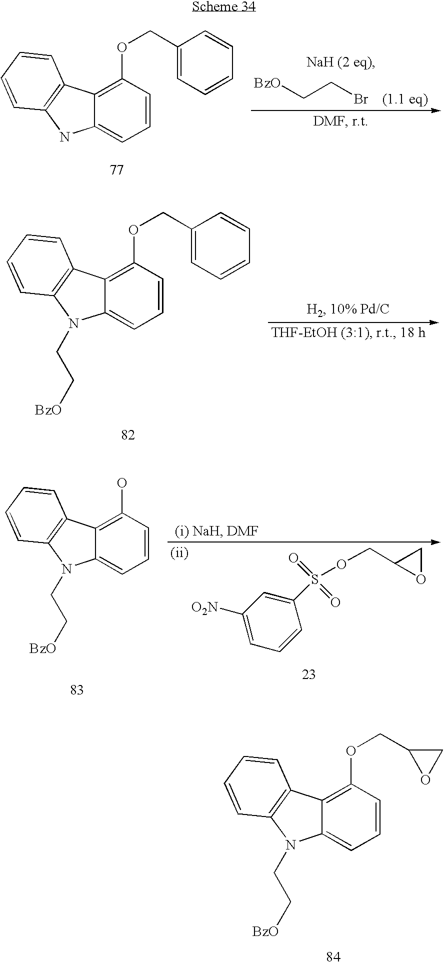

### Synthesis of Benzoic acid 2-(4-oxiranylmethoxy-carbazol-9-yl)-ethyl ester (84)

Benzoic acid 2-(4-oxiranylmethoxy-carbazol-9-yl)-ethyl ester (84) was synthesized using the procedure described for (80) except 2-bromoethyl benzoate was used in the second step instead of 2-bromoethyl methyl ether. A brown solid (261 mg, 85% yield, 88% pure by LC-MS and ¹H-nmr) was obtained.

### Example 35

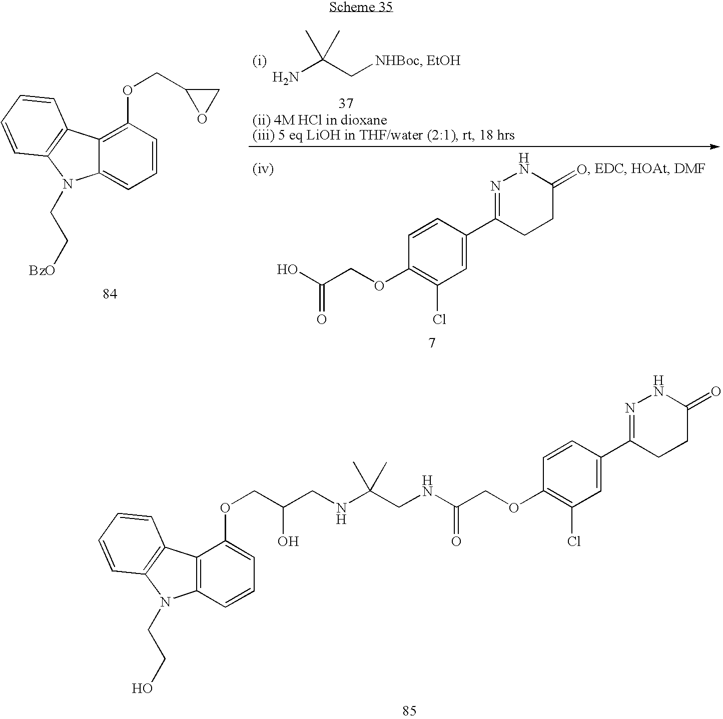

### Synthesis of 2-[2-Chloro-4-(6-oxo-1,4,5,6-tetrahydro-pyridazin-3-yl)-phenoxy]-N-(2-{2-hydroxy-3-[9-(2-hydroxy-ethyl)-9H-carbazol-4-yloxy]-propylamino}-2-methyl-propyl)-acetamide (85)

Compound 85 was synthesized via the procedure described for 49 but using 2-(4-oxiranylmethoxy-carbazol-9-yl)-ethyl benzoate (84) instead of (24), as shown in Scheme 35. A white solid (80 mg, 20% yield over 4 steps), 91% pure by LC-MS and ¹H-nmr was obtained. 2.5 min LC-MS (UV @215 nm: retention time=1.54 min., peak area=91%, TOF-ES⁺ with 25 eV cone voltage: m/z=636.49 (100%) & 638.44 (40%)). ¹H NMR: ([D₆]-DMSO, δ in ppm): 11.04 (1H, s), 8.37 (1H, d, J=7.6 Hz), 7.90 (1H, d, J=2.4 Hz), 7.82 (1H, m), 7.75 (1H, dd, J¹=8.4 Hz, J²=2.4 Hz), 7.70 (1H, d, J=8.4 Hz), 7.51 (1H, m), 7.41 (1H, t, J=8.4 Hz), 7.31-7.23 (2H, m), 7.21 (1H, d, J=8.8 Hz), 6.82 (1H, d, J=8.4 Hz), 5.26 (1H, br s), 5.00 (1H, t, J=6.0 Hz), 4.76 (2H, s), 4.52 (2H, t, J=6.4 Hz), 4.31 (2H, m), 4.17 (1H, br s), 3.88 (2H, q, J=6.0 Hz), 3.26 (2H, d, J=5.6 Hz), 3.03-2.93 (3H, m), 2.86 (1H, m), 2.52 (2H, t, J=9.2 Hz), 1.15 (6H, s).

### Example 36

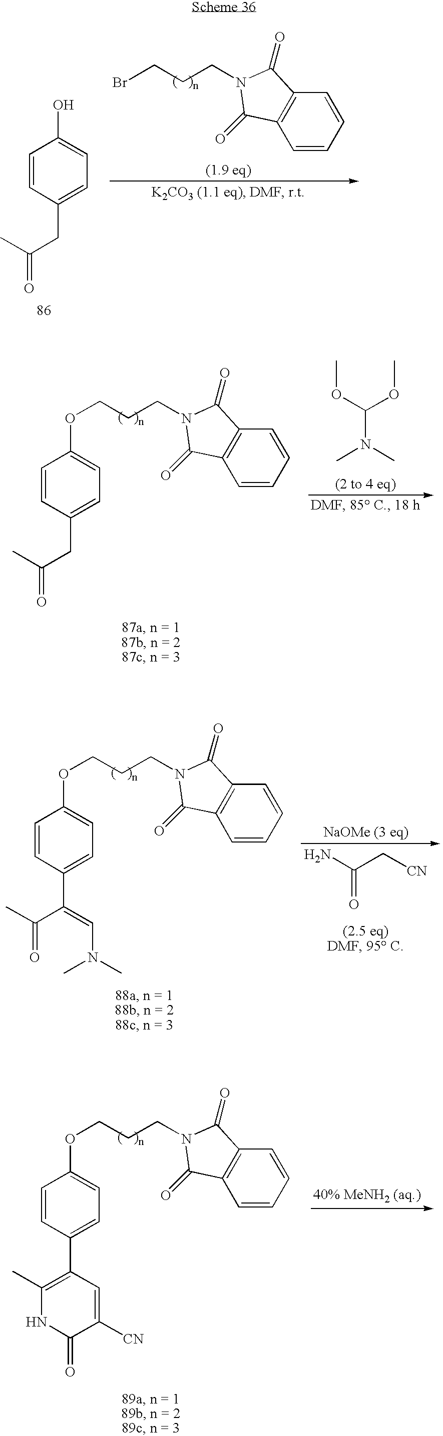

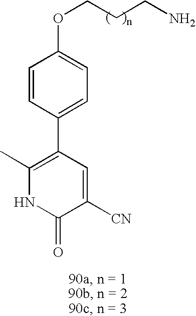

### Synthesis of 2-{3-[4-(2-oxo-propyl)-phenoxy]-propyl}-isoindole-1,3-dione (87a)

To a stirred solution of 4-hydroxyphenyl acetone (86, 1.00 g, 6.67 mmol) in N,N-dimethylformamide (20 mL) were added N-(3-bromopropyl)phthalimide (1.70 g, 6.34 mmol) and potassium carbonate (1.02 g, 7.34 mmol). The reaction mixture was stirred at ambient temperature for 24 hours. The reaction mixture was quenched with water (100 mL) and extracted with ethyl acetate (3×100 mL). The combined organic layers were washed with aqueous sodium hydroxide (2N, 200 mL) and saturated brine (3×100 mL). The organic layers were dried over sodium sulphate and evaporated to dryness. 2-{3-[4-(2-oxo-propyl)-phenoxy]-propyl}-isoindole-1,3-dione (87a) was isolated as a brown solid (1.92 g, 86% yield, 90% pure by LC-MS and ¹H-nmr).

### Synthesis of 2-{3-[4-(1-dimethylaminomethylene-2-oxo-propyl)-phenoxy]-propyl}-isoindole-1,3-dione (88a)

To a stirred solution of 2-{3-[4-(2-oxo-propyl)-phenoxy]-propyl}-isoindole-1,3-dione (87a, 1.00 g, 2.96 mmol) in N,N-dimethylformamide (30 mL) was added N,N-dimethylformamide dimethylacetal (1.41 g, 11.86 mmol). The reaction mixture was then heated to 85° C. and stirred at this temperature for 18 h. The reaction mixture was allowed to cool to ambient temperature and excess solvent and reagents were removed under reduced pressure to give crude 2-{3-[4-(1-dimethylaminomethylene-2-oxo-propyl)-phenoxy]-propyl}-isoindole-1,3-dione (88a) as a brown oil which was used in the following step without further purification.

### Synthesis of 5-{4-[3-(1,3-dioxo-1,3-dihydro-isoindol-2-yl)-propoxy]-phenyl}-6-methyl-2-oxo-1,2-dihydro-pyridine-3-carbonitrile (89a)

To a stirred solution of sodium hydride (60% dispersion in mineral oil, 356 mg, 8.89 mmol) in N,N-dimethylformamide (15 mL) was added dropwise at 0° C. a solution of crude 2-{3-[4-(1-dimethylaminomethylene-2-oxo-propyl)-phenoxy]-propyl}-isoindole-1,3-dione (88a) from the previous step, 2-cyano-acetamide (628 mg, 7.41 mmol) and methanol (360 μL, 8.89 mmol) in N,N-dimethylformamide (10 mL). The reaction mixture was then heated to 95° C. and stirred at this temperature for 4 days. Following this, the reaction mixture was allowed to cool to ambient temperature and the solvent was removed under reduced pressure. The residue was hydrolysed with aqueous saturated ammonium chloride solution (50 mL). The precipitated solid was collected by filtration with suction, rinsed with water and diethyl ether. The residue was then dry-loaded and purified by flash column chromatography over 165 g Silica gel eluting with a gradient of dichloromethane/ethyl acetate to neat ethyl acetate. Fractions with R_{f}=0.40 (ethyl acetate) were combined and concentrated under reduced pressure to afford 5-{4-[3-(1,3-dioxo-1,3-dihydro-isoindol-2-yl)-propoxy]-phenyl}-6-methyl-2-oxo-1,2-dihydro-pyridine-3-carbonitrile (89a) as a brown solid (368 mg, 30% yield, >95% pure by LC-MS and ¹H-nmr).

### Synthesis of 5-[4-(3-amino-propoxy)-phenyl]-6-methyl-2-oxo-1,2-dihydro-pyridine-3-carbonitrile (90a)

5-{4-[3-(1,3-Dioxo-1,3-dihydro-isoindol-2-yl)-propoxy]-phenyl}-6-methyl-2-oxo-1,2-dihydro-pyridine-3-carbonitrile (89a, 295 mg, 0.71 mmol) was dissolved with stirring in 40% w/v methylamine aqueous solution (70 mL, 903 mmol methylamine). The reaction mixture was then stirred at ambient temperature for 18 h. Following this, the solvent was removed under reduced pressure until a slurry was obtained (10 mL of solvent remaining). The white precipitate was then filtered off, rinsed with water (20 mL) and diethyl ether and dried under vacuum to give 5-[4-(3-amino-propoxy)-phenyl]-6-methyl-2-oxo-1,2-dihydro-pyridine-3-carbonitrile (90a) as a white solid (131 mg, 65% yield, >95% pure by LC-MS and ¹H-nmr).

### Example 37

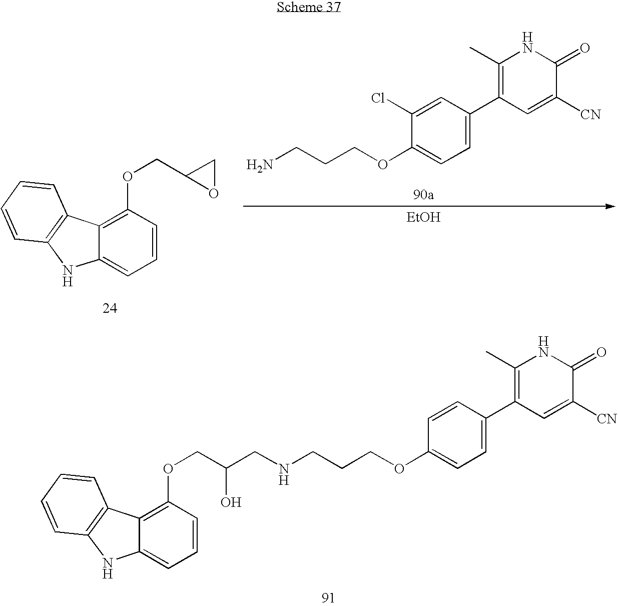

### Synthesis of 5-(4-{3-[3-(9H-carbazol-4-yloxy)-2-hydroxy-propylamino]-propoxy}-phenyl)-6-methyl-2-oxo-1,2-dihydro-pyridine-3-carbonitrile (91)

Compound 91 was synthesized via the procedure described for 64 using 5-[4-(3-amino-propoxy)-phenyl]-6-methyl-2-oxo-1,2-dihydro-pyridine-3-carbonitrile (90a) instead of 6-[4-(3-amino-propoxy)-3-chloro-phenyl]-4,5-dihydro-2H-pyridazin-3-one (28). A colorless powder (49 mg, 19% yield, 100% pure by LC-MS and ¹H-nmr) was obtained. 2.5 min LC-MS (UV @215 nm: retention time=1.59 min., peak area=100%, TOF-ES⁺ with 25 eV cone voltage: m/z=523.46 (100%) & 524.44 (40%). ¹H NMR: ([D₆]-DMSO, δ in ppm): 11.11 (1H, s), 8.08 (1H, d, J=8.0 Hz), 7.85 (1H, s), 7.31 (1H, m), 7.22-7.12 (2H, m), 7.08 (1H, m), 6.99 (1H, t, J=7.6 Hz), 6.93 (1H, d, J=8.4 Hz), 6.79 (1H, m), 6.54 (1H, d, J=8.4 Hz), 4.08-3.93 (3H, m), 3.90 (2H, t, J=6.8 Hz), 2.75 (1H, dd, J¹=12.4 Hz, J²=4.4 Hz), 2.74-2.58 (3H, m), 2.09 (3H, s), 1.75 (2H, m).

### Example 38

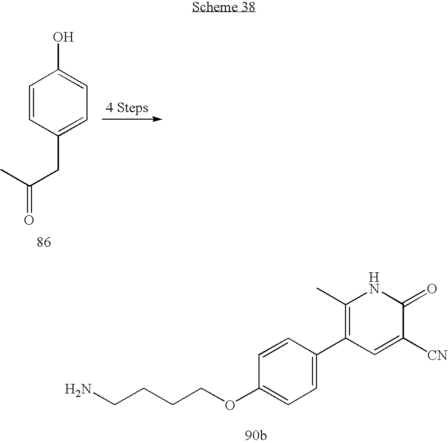

### Synthesis of 5-[4-(4-amino-butoxy)-3-chloro-phenyl]-6-methyl-2-oxo-1,2-dihydro-pyridine-3-carbonitrile (90b)

5-[4-(4-Amino-butoxy)-3-chloro-phenyl]-6-methyl-2-oxo-1,2-dihydro-pyridine-3-carbonitrile (90b) was synthesized according to the procedure used for (90a) except N-(4-bromobutyl)phthalimide was used in the first step instead of N-(3-bromopropyl)phthalimide. 5-[4-(4-Amino-butoxy)-3-chloro-phenyl]-6-methyl-2-oxo-1,2-dihydro-pyridine-3-carbonitrile (90b) was isolated as a colorless powder (252 mg, 11% yield, >95% pure by LC-MS and ¹H-nmr).

### Example 39

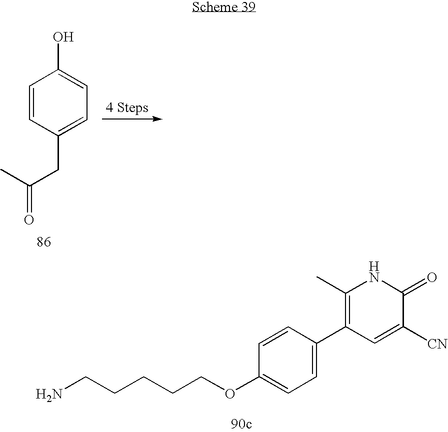

### Synthesis of 5-[4-(5-amino-pentyloxy)-3-chloro-phenyl]-6-methyl-2-oxo-1,2-dihydro-pyridine-3-carbonitrile (90c)

5-[4-(5-Amino-pentyloxy)-3-chloro-phenyl]-6-methyl-2-oxo-1,2-dihydro-pyridine-3-carbonitrile (90c) was synthesized according to the procedure used for (90a) except N-(5-bromopentyl)phthalimide was used in the first step instead of N-(3-bromopropyl)phthalimide. 5-[4-(5-Amino-pentyloxy)-3-chloro-phenyl]-6-methyl-2-oxo-1,2-dihydro-pyridine-3-carbonitrile (90c) was isolated as a colorless powder (320 mg, 17% yield, >95% pure by LC-MS and ¹H-nmr).

### Example 40

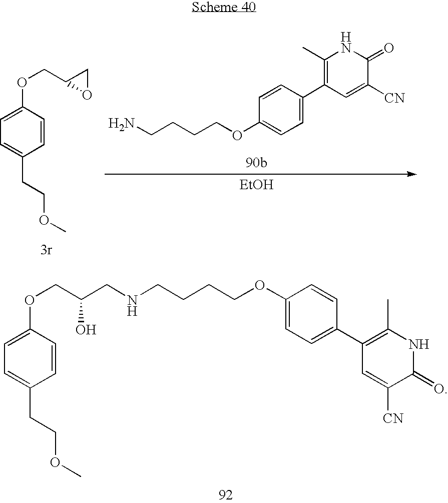

### Synthesis of 6-[4-(3-{(S)-2-hydroxy-propylamino]-butoxy}-phenyl)-4,5-dihydro-2H-pyridazin-3-one (92)

Compound 92 was synthesized via the procedure described for 44 except 5-[4-(4-amino-butoxy)-3-chloro-phenyl]-6-methyl-2-oxo-1,2-dihydro-pyridine-3-carbonitrile (90b) was used instead of 6-[4-(2-amino-butoxy)-3-chloro-phenyl]-4,5-dihydro-2H-pyridazin-3-one (43). A colorless solid (17 mg, 22% yield), 98% pure by LC-MS and ¹H-nmr was obtained. 2.5 min LC-MS (UV @215 nm: retention time=1.57 min., peak area=98%, TOF-ES⁺ with 25 eV cone voltage: m/z=506.51 (100%) & 507.50 (40%)). ¹H NMR: (CDCl₃, TMS internal standard, δ in ppm): 7.67 (1H, s), 7.04 (4H, m), 6.85 (1H, d, J=9.2 Hz), 6.77 (2H, d, J=8.8 Hz), 4.10 (1H, m), 3.97-3.87 (4H, m), 3.49 (2H, t, J=7.2 Hz), 3.28 (3H, s), 2.93 (1H, m), 2.87-2.71 (5H, m), 2.32 (3H, s), 1.86-1.77 (2H, m), 1.77-1.68 (2H, m).

### Example 41

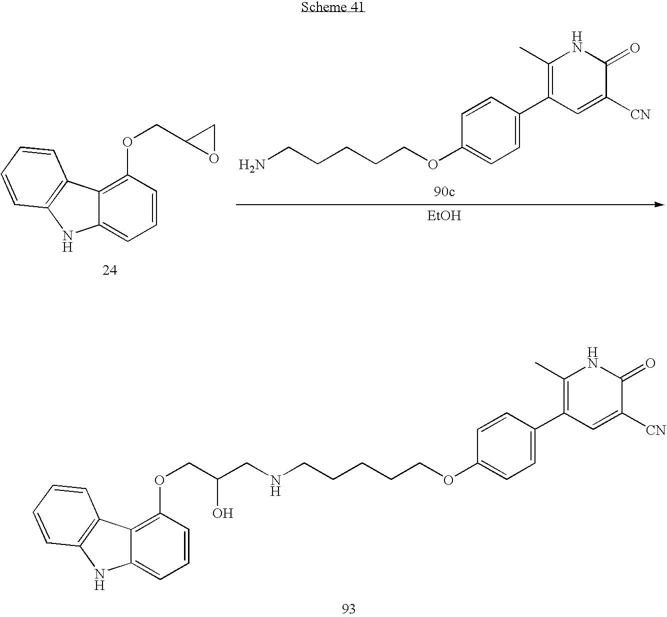

### Synthesis of 5-(4-{5-[3-(9H-carbazol-4-yloxy)-2-hydroxy-propylamino]-pentyloxy}-3-chloro-phenyl)-6-methyl-2-oxo-1,2-dihydro-pyridine-3-carbonitrile (93)

Compound 93 was synthesized via the procedure described for 91 using 5-[4-(5-amino-pentyloxy)-3-chloro-phenyl]-6-methyl-2-oxo-1,2-dihydro-pyridine-3-carbonitrile (90c) instead of 5-[4-(3-amino-propoxy)-phenyl]-6-methyl-2-oxo-1,2-dihydro-pyridine-3-carbonitrile (90a). A colorless solid (16 mg, 6% yield, 100% pure by LC-MS and ¹H-nmr) was obtained. 2.5 min LC-MS (UV @215 nm: retention time=1.65 min., peak area=100%, TOF-ES⁺ with 25 eV cone voltage: m/z=551.52 (100%) & 552.50 (40%). ¹H NMR: ([D₆]-DMSO, δ in ppm): 11.34 (1H, s), 8.29 (1H, d, J=8.0 Hz), 8.08 (1H, s), 7.52 (1H, d, J=8.4 Hz), 7.44-7.28 (4H, m), 7.21 (1H, t, J=8.0 Hz), 7.15 (1H, d, J=8.4 Hz), 7.02 (2H, m), 6.76 (1H, d, J=8.4 Hz), 4.24 (3H, s), 4.02 (2H, t, J=6.8 Hz), 3.04 (1H, m), 2.92 (1H, m), 2.77 (2H, m), 2.31 (3H, s), 1.78 (2H, m), 1.67-1.58 (2H, m), 1.57-1.49 (2H, m).

### Example 42

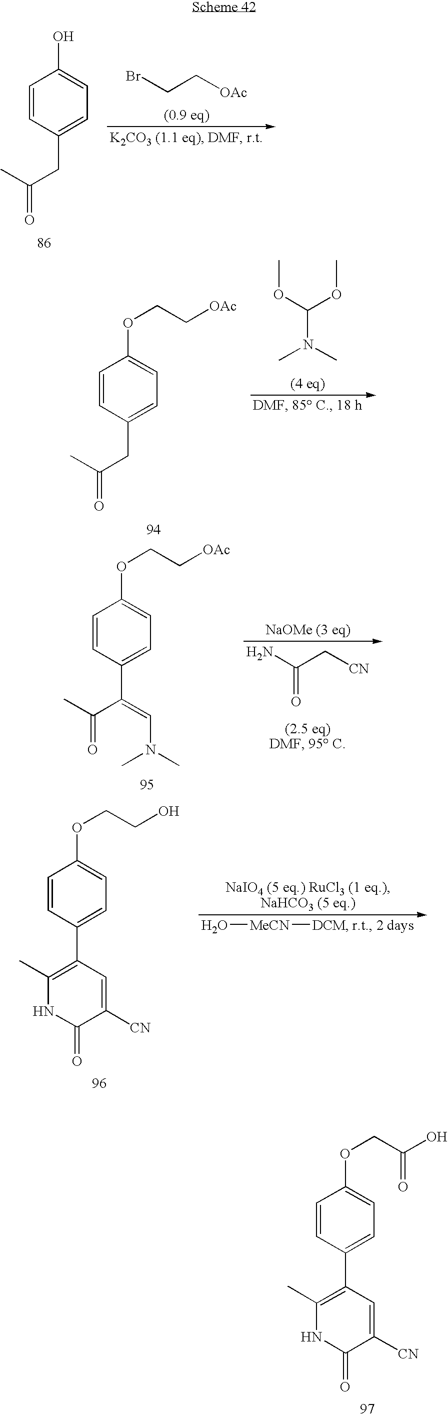

### Synthesis of acetic acid 2-[4-(2-oxo-propyl)-phenoxy]-ethyl ester (94)

Acetic acid 2-[4-(2-oxo-propyl)-phenoxy]-ethyl ester (94) was synthesized according to the procedure used for (87a) except 2-bromoethyl acetate was used in the first step instead of N-(3-bromopropyl)phthalimide. Acetic acid 2-[4-(2-oxo-propyl)-phenoxy]-ethyl ester (94) was isolated as a brown oil (6.82 g, 71% yield, 82% pure by LC-MS and ¹H-nmr).

### Synthesis of acetic acid 2-[4-(1-dimethylaminomethylene-2-oxo-propyl)-phenoxy]-ethyl ester (95)

Acetic acid 2-[4-(1-dimethylaminomethylene-2-oxo-propyl)-phenoxy]-ethyl ester (95) was synthesized according to the procedure used for (88a) except 2-bromoethyl acetate was used in the first step instead of N-(3-bromopropyl)phthalimide. Acetic acid 2-[4-(1-dimethylaminomethylene-2-oxo-propyl)-phenoxy]-ethyl ester (95) was isolated as a brown oil. It was used in the following step without any further purification.

### Synthesis of 5-[4-(2-hydroxy-ethoxy)-phenyl]-6-methyl-2-oxo-1,2-dihydro-pyridine-3-carbonitrile (96)

Acetic acid 2-[4-(1-dimethylaminomethylene-2-oxo-propyl)-phenoxy]-ethyl ester (95) was synthesized according to the procedure used for (89a) except 2-bromoethyl acetate was used in the first step instead of N-(3-bromopropyl)phthalimide. 5-[4-(2-Hydroxy-ethoxy)-phenyl]-6-methyl-2-oxo-1,2-dihydro-pyridine-3-carbonitrile (96) was isolated as a brown solid (725 mg, 63% yield, 97% pure by LC-MS and ¹H-nmr).

### Synthesis of [4-(5-cyano-2-methyl-6-oxo-1,6-dihydro-pyridin-3-yl)-phenoxy]-acetic acid (97)

To a stirred solution of 5-[4-(2-hydroxy-ethoxy)-phenyl]-6-methyl-2-oxo-1,2-dihydro-pyridine-3-carbonitrile (96, 500 mg, 1.85 mmol) in a mixture of distilled water-acetonitrile-dichloromethane (2:1:1 by volume) (20 mL) under nitrogen at 0° C. were added successively sodium hydrogenocarbonate (777 mg, 9.25 mmol), sodium periodate (1.58 g, 7.40 mmol) and ruthenium trichloride (192 mg, 0.92 mmol). The reaction mixture was stirred at ambient temperature for 3 hours, more sodium periodate (398 mg, 1.85 mmol) and ruthenium trichloride (192 mg, 0.92 mmol) were added and the reaction mixture was stirred at ambient temperature for 2 more days. The organic solvents were then removed under reduced pressure and the aqueous residue was treated with an aqueous sodium hydroxide solution (2N, 25 mL) and extracted with tert-butyl methyl ether (2×30 mL). The aqueous layer was acidified to pH 2 with hydrochloric acid solution (1N) and extracted with a mixture of ethyl acetate/methanol (9:1) (4×50 mL). The combined organic layers were dried over magnesium sulphate and removed under reduced pressure. The residue was dry loaded and purified by flash column chromatography over silica gel (25 g) eluting with a gradient of ethyl acetate/methanol. Fractions with R^{f}=0.60 (ethyl acetate/methanol (1:1)) were combined and concentrated under reduced pressure to give [4-(5-cyano-2-methyl-6-oxo-1,6-dihydro-pyridin-3-yl)-phenoxy]-acetic acid (97) as a colorless solid (145 mg, 24% yield, 90% pure by LC-MS and ¹H-nmr).

### Example 43

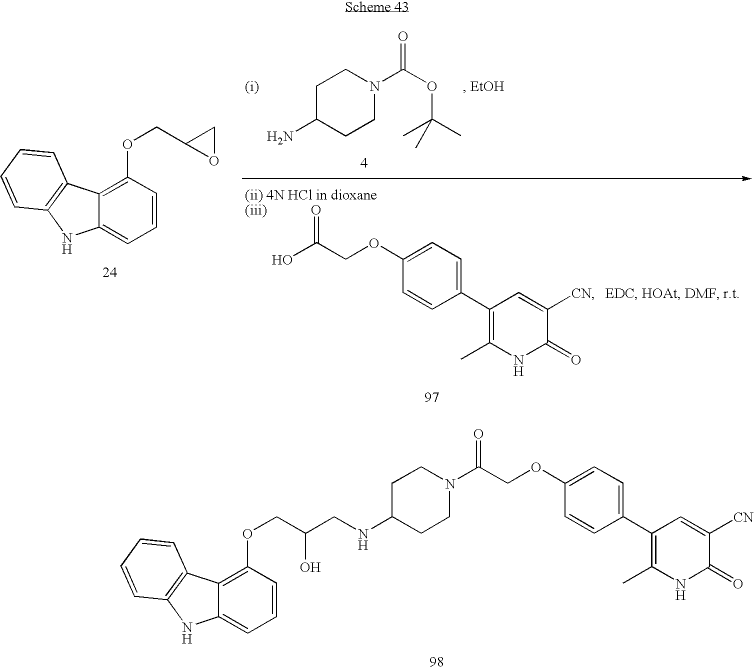

### Synthesis of 5-[4-(2-{4-[3-(9H-carbazol-4-yloxy)-2-hydroxy-propylamino]-piperidin-1-yl}-2-oxo-ethoxy)-phenyl]-6-methyl-2-oxo-1,2-dihydro-pyridine-3-carbonitrile (98)

Compound 98 was synthesized via the procedure described for 63 using [4-(5-cyano-2-methyl-6-oxo-1,6-dihydro-pyridin-3-yl)-phenoxy]-acetic acid (97) instead of [2-chloro-4-(6-oxo-1,4,5,6-tetrahydro-pyridazin-3-yl)-phenoxy]-acetic acid (7). A colorless solid (50 mg, 30% yield, 100% pure by LC-MS and ¹H-nmr) was obtained. 2.5 min LC-MS (UV @215 nm: retention time=1.54 min., peak area=100%, TOF-ES⁺ with 25 eV cone voltage: m/z=606.51 (100%) & 607.51 (40%). ¹H NMR: ([D₆]-DMSO, δ in ppm): 11.25 (1H, s), 8.21 (1H, d, J=8.4 Hz), 8.02 (1H, s), 7.45 (1H, m), 7.37-7.22 (4H, m), 7.14 (1H, t, J=8.4 Hz), 7.07 (1H, d, J=8.0 Hz), 6.94 (2H, m), 6.69 (1H, d, J=8.0 Hz), 5.15 (1H, br s), 4.83 (2H, m), 4.21-4.05 (4H, m), 3.76 (1H, m), 3.08 (1H, m), 2.93 (1H, dd, J¹=12.4 Hz, J²=4.8 Hz), 2.85-2.66 (3H, m), 2.24 (3H, s), 1.85 2(2H, m), 1.35-1.23 (1H, m), 1.21-1.09 (1H, m).

### Example 44

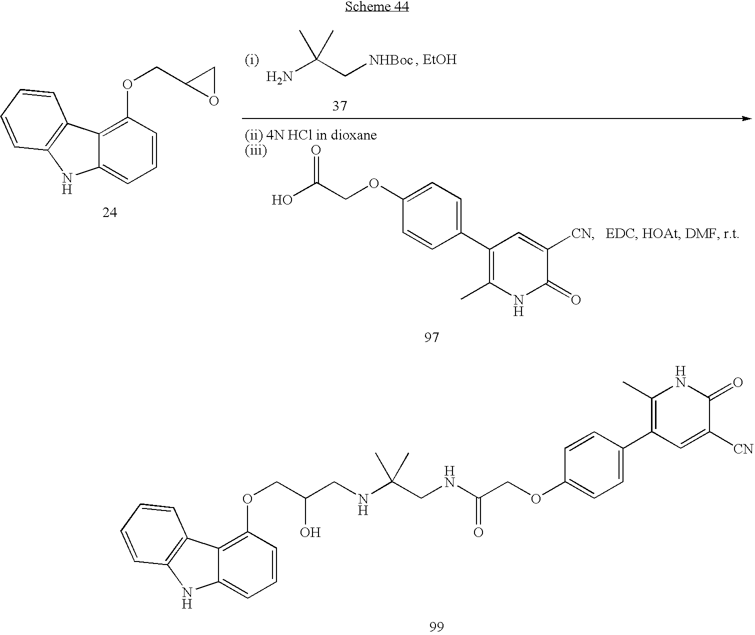

### Synthesis of N-{2-[3-(9H-carbazol-4-yloxy)-2-hydroxy-propylamino]-2-methyl-propyl}-2-[2-chloro-4-(5-cyano-2-methyl-6-oxo-1,6-dihydro-pyridin-3-yl)-phenoxy]-acetamide (99)

Compound 99 was synthesized via the procedure described for 49 using [4-(5-cyano-2-methyl-6-oxo-1,6-dihydro-pyridin-3-yl)-phenoxy]-acetic acid (97) instead of [2-chloro-4-(6-oxo-1,4,5,6-tetrahydro-pyridazin-3-yl)-phenoxy]-acetic acid (7). A colorless solid (41 mg, 24% yield, 100% pure by LC-MS and ¹H-nmr) was obtained. 2.5 min LC-MS (UV @215 nm: retention time=1.61 min., peak area=100%, TOF-ES⁺ with 25 eV cone voltage: m/z=594.52 (100%) & 595.49 (40%). ¹H NMR: ([D₆]-DMSO, δ in ppm): 11.18 (1H, s), 8.16 (1H, d, J=8.4 Hz), 7.88 (1H, s), 7.64 (1H, m), 7.38 (1H, d, J=8.8 Hz), 7.27 (1H, m), 7.20 (1H, t, J=8.4 Hz), 7.13 (2H, m), 7.04 (1H, m), 7.00 (1H, d, J=8.0 Hz), 6.90 (2H, m), 6.61 (1H, d, J=8.0 Hz), 5.07 (1H, br s), 4.48 (2H, s), 4.11 (2H, m),3.95 (1H, m), 3.06 (2H, d, J=5.6 Hz), 2.75 (1H, m), 2.70-2.61 (1H, m), 2.12 (3H, s), 0.91 (6H, s).

### Example 45

### 6-[4-(2-{4-[3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (126) was Synthesized According to the Method of Scheme 48.

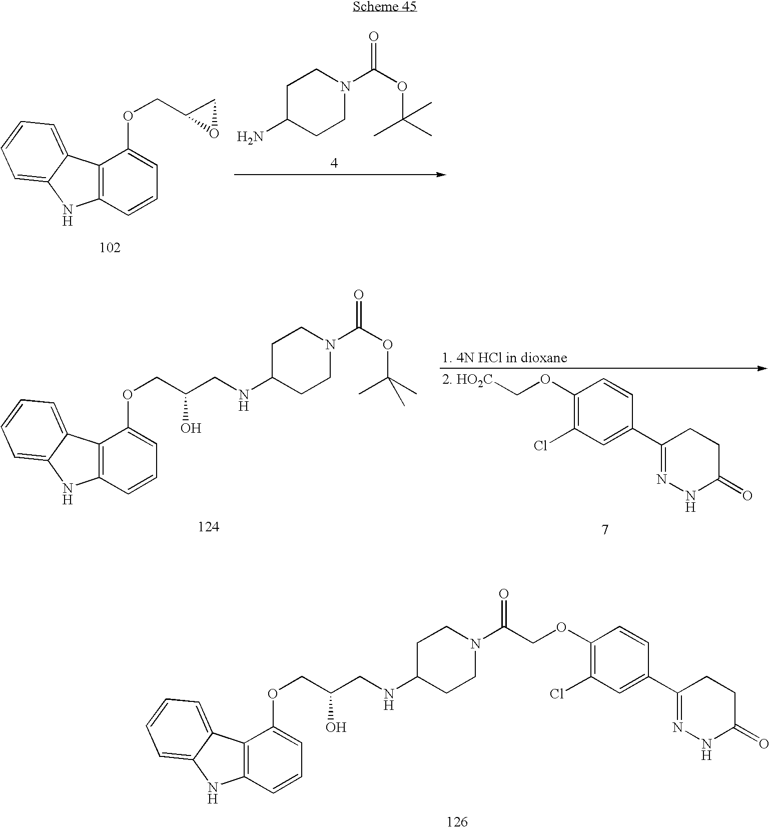

### 4-[3-(9H-Carbazol-4-yloxy)-2-hydroxy-propylamino]-piperidine-1-carboxylic acid tert-butyl ester (124)

To a stirred suspension of 4-oxiranylmethoxy-9H-carbazole (102, 300 mg, 1.25 mmol) in ethanol (15 mL) was added 4-amino-piperidine-1-carboxylic acid tert-butyl ester (4), 502 mg, 2.81 mmol). The mixture was stirred for 4 h under reflux and the solvent was then removed under reduced pressure. The residue was purified by flash column chromatography on a Biotage® system (40-M column) eluting with a gradient of 1-5% methanol in dichloromethane. 4-[3-(9H-Carbazol-4-yloxy)-2-hydroxy-propylamino]-piperidine-1-carboxylic acid tert-butyl ester (124) was isolated as a brown gum (312 mg, 57% yield, 92% pure by LC-MS and ¹H-nmr).

### 6-[4-(2-{4-[3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (126)

To a stirred solution of 4-[3-(9H-carbazol-4-yloxy)-2-hydroxy-propylamino]-piperidine-1-carboxylic acid tert-butyl ester (124, 312 mg, 0.709 mmol) in methanol (5 mL) was added hydrogen chloride in 1,4-dioxane (4N, 2.5 mL, 10.0 mmol). The mixture was stirred at ambient temperature for 16 h and the solvent was then removed under reduced pressure. The residue was dissolved in methanol (30 mL) and Ambersep® 900 (carbonate form, 2.5 g, 3.75 mmol) was added. The mixture was shaken at ambient temperature for 3 h and then filtered. The filter residue was rinsed with methanol (3×15 mL) and the combined filtrates were evaporated to dryness. The residue was re-dissolved in N,N-dimethylformamide (4 mL). Separately, a suspension of (3-dimethylamino-propyl)-ethyl-carbodiimide hydrochloride (EDC.HCl, 270 mg, 1.41 mmol), [1,2,3]triazolo[4,5-b]pyridin-3-ol (HOAt, 96 mg, 0.70 mmol) and [2-chloro-4-(6-oxo-1,4,5,6-tetrahydro-pyridazin-3-yl)-phenoxy]-acetic acid (7), 199 mg, 0.70 mmol) in N,N-dimethylformamide (4 mL) was stirred at ambient temperature for 30 minutes until a clear solution. Both solutions were combined and the mixture was stirred for 16 h at ambient temperature then diluted with ethyl acetate (30 mL) and water (60 mL). The mixture was adjusted to pH=12 with 2N aqueous sodium hydroxide solution. The aqueous phase was extracted with ethyl acetate (3×30 mL). The combined organic layers were washed with saturated brine (4×100 mL), dried over sodium sulphate and evaporated to dryness. The residue was purified by flash column chromatography on silica gel (10 g) eluting with dichloromethane/methanol 10:1 to give 6-[4-(2-{4-[3-(9H-carbazol-4-yloxy)-2-hydroxy-propylamino]-piperidin-1-yl}-2-oxo-ethoxy)-3-chloro-phenyl]-4,5-dihydro-2H-pyridazin-3-one (126) as colorless powder (220 mg, 51% yield, 99% pure by LC-MS and ¹H-nmr). 2.5 min LC-MS (UV @215 nm: retention time=1.12 min., peak area=100%, TOF-ES⁺ with 25 eV cone voltage: m/z=604 (100%) & 606 (50%)). ¹H NMR: ([D₆]-DMSO, δ in ppm): 11.23 (1H, s), 10.88 (1H, s), 8.20 (1H, d, J=7.78 Hz), 7.76 (1H, d, J=2.29 Hz), 7.60 (1H, dd, J¹=8.74 Hz, J²=2.24 Hz), 7.43 (1H, d, J=8.05 Hz), 7.34-7.26 (2H, m), 7.12 (1H, m), 7.05 (1H, d, J=7.96 Hz), 7.02 (1H, d, J=8.87 Hz), 6.67 (1H, d, J=7.87 Hz), 5.11 (1H, m), 4.99 (2H, m), 4.15 (2H, m), 4.05 (2H, m), 3.74 (1H, m), 3.07 (1H, m), 2.89 (3H, m), 2.83-2.65 (3H, br m), 2.41 (2H, t, J=8.28 Hz), 1.82 (2H, m), 1.30 (1H, m), 1.13 (1H, m).

### Example 46

### 6-{3-Chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (8r) was synthesized according to the method of Scheme 46.

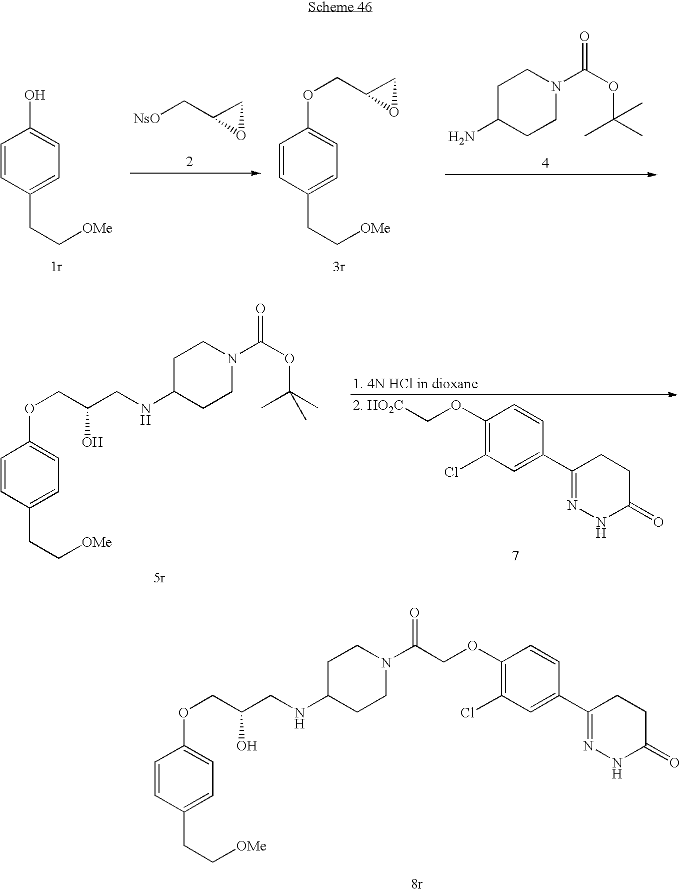

### 2-[4-(2-Methoxy-ethyl)-phenoxymethyl]-oxirane (3r)

To a stirred solution of 4-(2-methoxy-ethyl)-phenol ((1r), 200 mg, 1.31 mmol) and 3-nitro-benzenesulfonic acid oxiranylmethyl ester ((2), 342 mg, 1.31 mmol) in butan-2-one was added potassium carbonate (200 mg, 1.45 mmol). The mixture was stirred under reflux for 18 h and the solvent then removed under reduced pressure. The residue was separated between dichloromethane (25 mL) and water (25 mL). The aqueous phase was extracted with dichloromethane (2×25 mL). The combined organic layers were washed with aqueous sodium hydroxide solution (2N, 25 mL) and saturated brine (25 mL), dried over sodium sulphate and evaporated to dryness to give 2-[4-(2-methoxy-ethyl)-phenoxymethyl]-oxirane (3r) as a yellow oil (250 mg, 92% yield, 95% pure by LC-MS and ¹H-nmr).

### 4-{2-Hydroxy-3-[4-(2-methoxy-ethyl)-phenoxy]-propylamino}-piperidine-1-carboxylic acid tert-butyl ester (5r)

To a stirred solution of 2-[4-(2-methoxy-ethyl)-phenoxymethyl]-oxirane ((3r), 250 mg, 1.20 mmol) in ethanol (30 mL) was added 4-amino-piperidine-1-carboxylic acid tert-butyl ester ((4), 520 mg, 2.60 mmol). The mixture was stirred under reflux for 4 h and the solvent then removed under reduced pressure. The residue was purified by flash column chromatography on silica gel (15 g), eluting with dichloromethane/methanol 100:5. 4-{2-Hydroxy-3-[4-(2-methoxy-ethyl)-phenoxy]-propylamino}-piperidine-1-carboxylic acid tert-butyl ester (5r) was isolated as a colorless oil which solidified on standing (306 mg, 62% yield, >95% pure by LC-MS and ¹H-nmr).

### 6-{3-Chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (8r)

To a stirred solution of 4-{2-hydroxy-3-[4-(2-methoxy-ethyl)-phenoxy]-propylamino}-piperidine-1-carboxylic acid tert-butyl ester ((5r), 300 mg, 0.735 mmol) in 1,4-dioxane (2 mL) was added hydrogen chloride in 1,4-dioxane (4N, 3 mL, 12.0 mmol). The mixture was stirred at ambient temperature for 2 h and the solvent was then removed under reduced pressure. The residue was dissolved in methanol (30 mL) and Ambersep® 900 (carbonate form, 4.9 g, 7.35 mmol) was added. The mixture was shaken at ambient temperature for 2 h and then filtered. The filter residue was rinsed with methanol (2×15 mL) and the combined filtrates were evaporated to dryness. The residue was re-dissolved in N,N-dimethylformamide (7 mL). Separately, a suspension of (3-Dimethylamino-propyl)-ethyl-carbodiimide hydrochloride (EDC.HCl, 137 mg, 0.713 mmol), [1,2,3]Triazolo[4,5-b]pyridin-3-ol (HOAt, 39 mg, 0.285 mmol) and [2-chloro-4-(6-oxo-1,4,5,6-tetrahydro-pyridazin-3-yl)-phenoxy]-acetic acid ((7), 202 mg, 0.713 mmol) in N,N-dimethylformamide (7 mL) was stirred at ambient temperature for 5 minutes until a clear solution. Both solutions were combined and the mixture was stirred for 16 h at ambient temperature. After diluting with ethyl acetate (200 mL) the organic phase was first washed with a mixture of saturated potassium carbonate (45 mL), water (55 mL and methanol (15 mL) then with water (100 mL) and saturated brine (100 mL). The organic layer was dried over sodium sulphate and the solvent was removed under reduced pressure. The residue was purified by flash column chromatography on silica gel (20 g) eluting with dichloromethane/methanol 100:5 to give 6-{3-chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (8r) as a colorless powder (320 mg, 78% yield, 99% pure by LC-MS and ¹H NMR). 2.5 min LC-MS (UV @215 nm: retention time=1.18 min., peak area=100%, TOF-ES⁺ with 25 eV cone voltage: m/z=573 (100%) & 575 (50%)). ¹H NMR: (CDCl₃, TMS internal standard, δ in ppm): 8.66 (1 H, s), 7.80 (1H, d, J=2.20 Hz), 7.53 (1H, dd, J¹=8.80 Hz, J²=2.20 Hz), 7.13 (2H, d, J=8.56 Hz), 7.03 (1H, d, J=8.56 Hz), 6.83 (2H, d, J=8.56 Hz), 4.82 (2H, m), 4.38 (1H, d, J=13.45 Hz), 4.11-3.98 (2H, m), 3.95 (2H, m), 3.56 (2H, t, J=6.97 Hz), 3.35 (3H, s), 3.17 (1H, m), 2.96-2.85 (3H, m), 2.85-2.70 (4H, m), 2.60 (2H, m), 1.95 (2H, m), 1.38-1.18 (2H, m).

### Example 47

2′-(2-{4-[3-(2-Cyano-phenoxy)-2-hydroxy-propylamino]-piperidin-1-yl}-ethoxy)-2-methyl-6-oxo-1,6-dihydro-[3,4′]bipyridinyl-5-carbonitrile (137) is Synthesized According to the Method of Scheme 47.

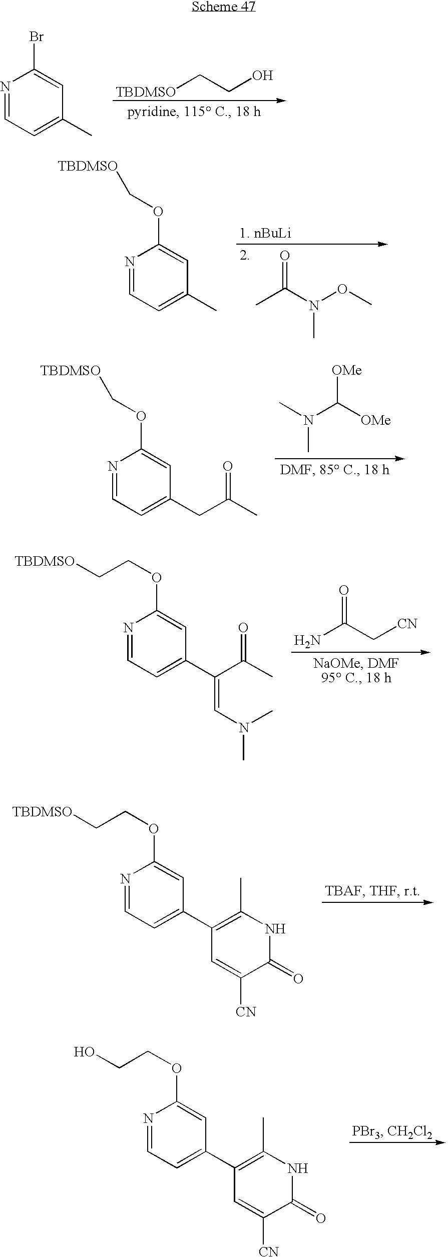

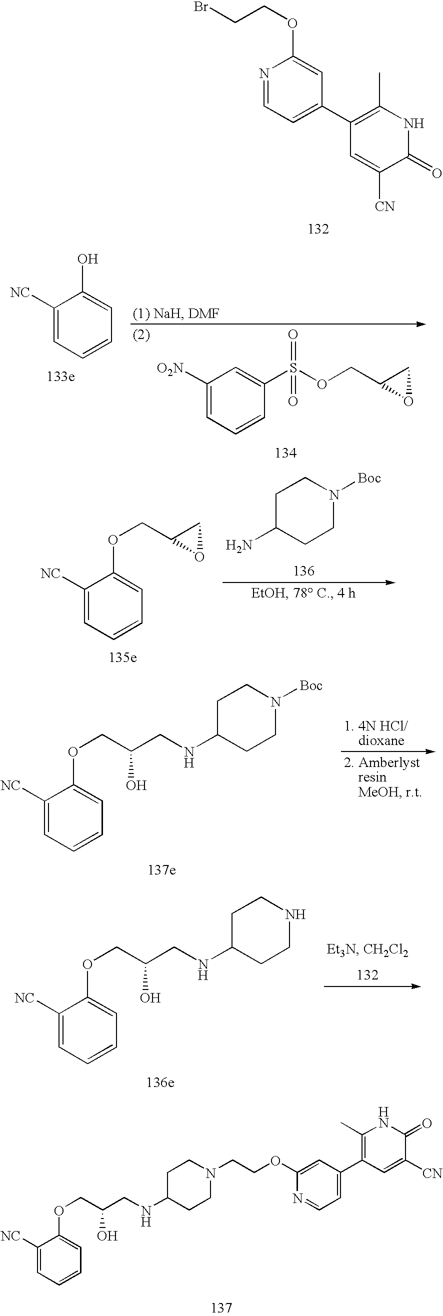

### Example 48

### SYNTHESIS OF 6-[4-(2-{3-[3-(9H-CARBAZOL-4-YLOXY)-2-HYDROXY-PROPYLAMINO]-PYRROLIDIN-1-YL}-2-OXO-ETHOXY)-3-CHLORO-PHENYL]-4,5-DIHYDRO-2H-PYRIDAZIN-3-ONE (139)

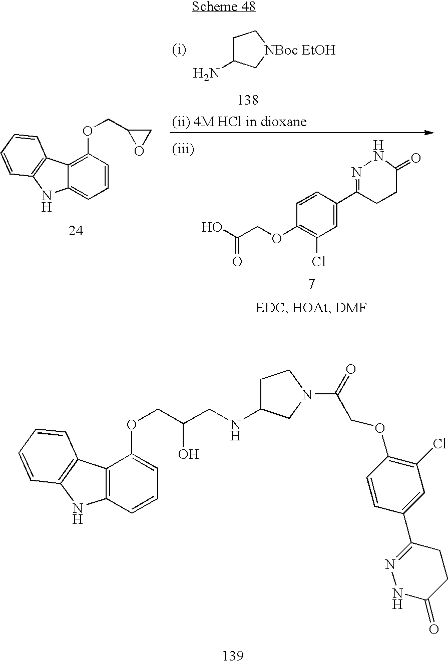

Compound 139 was synthesized via the procedure described for 49 but using 3-amino-pyrrolidine-1-carboxylic acid tert-butyl ester (138) instead of (2-amino-2-methyl-propyl)-carbamic acid tert-butyl ester (37). A colorless solid (230 mg, 59% yield for the last step), 100% pure by LC-MS and ¹H-nmr was obtained. 2.5 min LC-MS (UV @215 nm: retention time=1.71 min., peak area=100%, TOF-ES⁺ with 25 eV cone voltage: m/z=590.28 (100%) & 592.28 (40%)). ¹H NMR: ([D₆]-DMSO, δ in ppm): 11.25 (1H, s), 10.90 (1H, s), 8.22 (1H, m), 7.78 (1H, t, J=2.8 Hz,), 7.59 (1H, m), 7.44 (1H, dd, J¹=8.4 Hz, J²=2.4 Hz), 7.36-7.26 (2H, m), 7.15 (1H, t, J=8.4 Hz), 7.10-6.98 (2H, m), 6.69 (1H, d, J=8.4 Hz), 5.17 (1H, br m), 4.97-4.79 (2H, m), 4.23-4.05 (3H, m), 3.71-3.55 (1H, m), 3.54-3.19 (4H, m), 2.97-2.71 (4H, m), 2.41 (2H, m), 2.12-1.62 (3H, m).

### Example 49

### PDE-3 Inhibitory Activity

In vitro Assay for Measuring cAMP PDE-3 Inhibitory Activity

Human platelet cyclic AMP phosphodiesterase was prepared according to the method of Alvarez et al., Mol. Pharmacol. 29: 554 (1986). The PDE incubation medium contained 10 mM Tris-HCl buffer, pH 7.7, 10 mM MgSO₄, and 1 μM [³H]AMP (0.2 μCi) in a total volume of 1.0 mL. Test compounds were dissolved in DMSO immediately prior to addition to the incubation medium, and the resulting mixture was allowed to stand for 10 minutes prior to the addition of enzyme. Following the addition of PDE, the contents were mixed and incubated for 10 minutes at 30° C. Three assays each were performed for each of five test compound concentrations, the mean of the determinations (n=3) at each concentration was plotted, and IC₅₀ values were determined graphically. A number of compounds tested, including but not limited to, compounds 8d, 8l, 8e, 8m, 8g, 8c, 8j, 8f, 8h, 8a, 8k, 8o, 8b, 8i, 8p, 8r, 33, 22, 34, 17, 29, 35, 25, 13, 36, 40, 41, 49, 52, 42, 44, 46, 56, 57, 48, 63, 64, 68, 69, 70, 72a, and 75 had PDE3 inhibition IC₅₀ values less than 1 μM. Of these, compounds 8d, 8g, 8j, 8i, 22, 29, 25, 36, 41, 49, 52, 56, 64, 69, and 70 had PDE IC₅₀ inhibition values less than 100 nM.

### Example 50

### B-Adrenergic Receptor Binding Activity

β-Adrenergic receptor binding and blocking activity was evaluated by one or more of the methods below.

### Radioligand for Measuring Non-Specific β-Receptor Activity

Non-specific receptor binding was measured for each test compound for beta-receptors from rat cortical membranes, using [³H]DHA as the radioligand, as described in Riva and Creese, Mol. Pharmacol. 36:211 (1989) and Arango et al., Brain Res., 516:113 (1990). A number of compounds tested, including but not limited to 8d, 8e, 8m, 8g, 8c, 8j, 8f, 8h, 8a, 8k, 8b, 8i, 8p, 33, 22, 17, 29, 25,13, 36, 40, 41, 49, 52, 44, 46, 56, 57, 61, 62, 63, 64, 68, 69, 70, 72a, 72b, 139, 75, and 74 had IC₅₀ values less than 1 μM. Of these, compounds 8d, 8g, 8c, 8j, 8f, 8h, 8k, 8b, 8i, 8p, 22, 17, 29, 25, 40, 49, 52, 44, 56, 57, 61, 62, 63, 64, 68, 69, 70, 72a, 72b, 139, 75, and 74 had IC₅₀ values less than 100 nM.

### Radioligand for Measuring β₁-Receptor Affinity

β₂-Adrenergic receptor binding was measured in human recombinant beta-1 receptors expressed in CHO-REX16 cells, using [¹²⁵I] (-) iodocyanopindolol (2000 Ci/mmol) as the radioligand, as described in Kalaria et al., J. Neurochem. 53: 1772-81 (1998), and Minneman et al., Mol. Pharmacol. 16: 34-46 (1979). Compounds 49, 8d, 8g, 8c, 25, 41, and 44 had greater than 25% inhibition at 100 nM.

### Radioligand for Measuring β₂-Receptor Affinity

β₂-Adrenergic receptor binding was measured in human recombinant beta-2 receptors expressed in CHO-WT21 cells, using [¹²⁵I] (-) iodocyanopindolol (2000 Ci/mmol) as the radioligand, as described in Kalaria et al. (1998) and Minneman et al. (1979), supra. Compounds 8d, 8g, 8c, 8p, 25, and 49 had greater than 25% inhibition at 100 nM.

### Effect on β₂-Adrenergic Blocking Activity

Tracheal chains are prepared as described by Castillo and DeBeer, J. Pharm. Exp. Ther. 90: 104 (1947), suspended in tissue baths maintained at 37° C. containing Tyrodes solution gassed with 95% O₂-5% CO₂, and attached to an isometric force-displacement transducer. After an equilibration period of 2 hours, the preparations are induced to contract with carbachol (3×10⁻⁷ M), and relaxation is induced with cumulative dose response curves for isoproterenol first in the absence of and then in the presence of the test compound. A contact time of 10 minutes is allowed for all test compounds. Affinity constants are determined by comparing the shift in the dose-response curve for each test compound with that of isoproterenol (EC₅₀=2.3×0.2×10⁻⁸ M).

### Example 51

### Restoration of Calcium Homeostasis in Heart Tissue

### Effect on Contraction-Relaxation in Guinea Pig Papillary Muscle

Male guinea pigs (400-500 g) are killed by cervical dislocation and the hearts are quickly removed, immersed in ice-cold solution, and oxygenated in Kreb's solution containing 113.1 mM NaCl, 4.6 mM KCl, 2.45 mM CaCl₂, 1.2 mM MgCl₂, 22.0 mM NaH₂PO₄, and 10.0 mM glucose; pH 7.4 with 95% O₂−5% CO₂. The ventricles are opened and papillary muscles are removed with the chordae tendineae and a base of surrounding tissue intact. The tendinous ends of the muscles are ligated with silk thread, and the muscles are mounted in vertical, double-jacketed organ baths containing 10 mL of oxygenated Kreb's solution kept at 37° C. The tendinous end is attached to a Grass isometric force transducer, while a metal hook is inserted into the base of the muscle.

Following a 45 minute equilibration period under a 1 gram tension, control contractions are elicited by stimulating the muscle using stainless steel field electrodes at a frequency of 1.0 Hz, 2.0 ms duration. The amplitude of the stimulus is adjusted to be approximately 1.5 times the threshold amplitude sufficient to elicit a contraction of the tissues. Control contraction-relaxation cycles are recorded for 30 seconds continuously. Cumulative test drug concentrations are then injected directly into the bath while the tissue is being stimulated. Contraction-relaxation recordings are made continuously, for 30 seconds per test compound concentration. A series of washout contractions is recorded following a change of solution. Provided that the amplitude of contraction returns to that measured in control conditions, a single concentration of positive control is then tested on the tissue in the same manner as the test compound.

Contraction amplitude as well as the time courses of contraction and relaxation are quantified. All recordings are normalized against control values; statistical analysis of the results is made using t-tests or ANOVAs.

In vitro Effect on Contractility

The effect of the compounds of the present invention when administered alone and in combination of 100 nM isoproterenol on isolated cardiomyocytes was tested in isolated ventricular myocytes from rabbit hearts. Isoproterenol, a potent β-Adrenergic agonist, can produce large increases in cardiac contraction, calcium transient amplitude, and the rates of relaxation (acceleration of relaxation or lusitropic effect). The effects of isoproterenol are then antagonized with different concentrations of a compound of the present invention.

Cardiac myocytes were digested from healthy white New Zealand male rabbits (3-5 lbs), with enzymatic digestion. Briefly, each animal is anesthetized with ketamine (50 mg/kg) and xylazine (6 mg/kg)-IM injection in hind limb. Once the animal was sedated (˜10-15 min), 0.1-0.3 ml of pentobarbital was injected into the ear vein. The heart was exposed by a cut just below the rib-cage and bilateral thoracotomy and removed rapidly ensuring that aorta remains intact. The heart was immediately placed in oxygenated NT with Ca²⁺ placed on ice for rinsing the blood out, cleared from vessels and pericardium, cannulated and maintained at 37° C. The heart was retrogradedly perfused and tissue digested with collagenase and protease. Digested myocytes were subsequently stored in 0.1 mM Ca²⁺ normal tyrodes for further analyses. Sarcomere length changes upon treatment with test compounds were recorded at 37° C. in the presence of 2 mM calcium and analyzed with an IonOptix system. Sarcomere length data was acquired for each myocyte over an average of 10 beats duration, at pacing rates of 1, 2, and 3 Hz. Basal percent sarcomere shortening and length-frequency relation of each myocyte was evaluated, and serve as a measure of cellular viability.

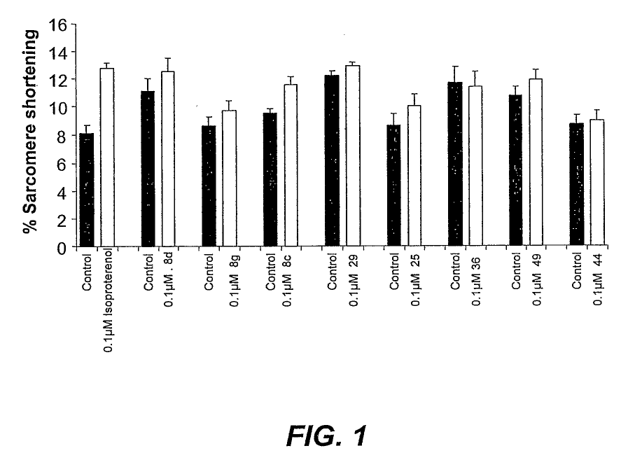

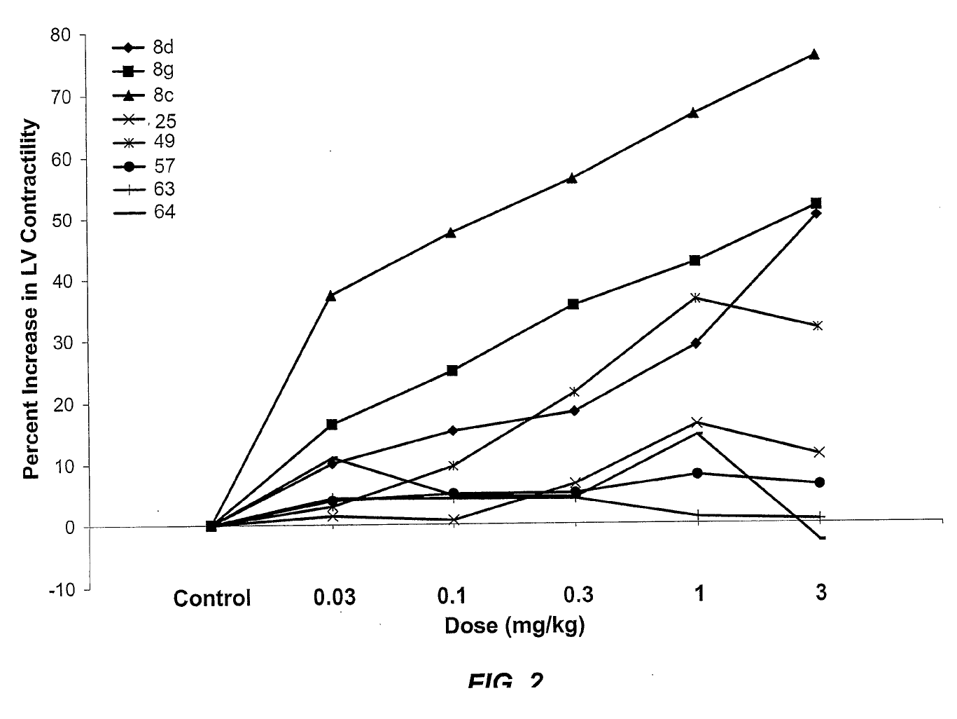

As shown in FIG. 1, all compounds tested increased sarcomere shortening significantly at 0.1 μM concentration at a pacing rate of 2 Hz. The addition of 0.1 μM isoproterenol did not increase contractility further, indicating complete beta-receptor antagonism by the test compounds.

In vivo Effect on Contractility and D-Adrenergic Antagonist Activity

Studies were performed on White New Zealand male rabbits (2-3 kg weight). Animals were initially anesthetized with ketamine (50 mg/kg; IM) and xylazine (6 mg/kg, IM). Subsequently, animals were intubated (via tracheotomy; 3 mm tube) and ventilated with 2% isoflurane (mixed in 95% O₂+5% CO₂). Each rabbit was instrumented for LV pressure (3F Millar catheter) through right carotid artery, arterial pressure (3F catheter, Cook Instruments) through left femoral artery, which was connected to a fluid-filled pressure transducer (BD Instruments), and ECG. Both pressure transducers were zeroed against atmospheric pressure, and calibrated before each study using an analog manometer. Upon completion of instrumentation, isoflurane was reduced to 1.25%, and the animal was covered for maintenance of body core temperature. Arterial and LV blood pressures (LVP) and the ECG signals were simultaneously digitally recorded on a PC. The recording system (Gould Instruments) and the corresponding software (Ponema, Gould Instruments) facilitated detailed calculation of various parameters directly from all signals and record on a separate file with a 1 sec resolution. Upon completion of the study protocol, rabbits were euthenized by isoflurane overdose (5% for at least 1 minute) and cardiac arrest through IV infusion of 5 ml 3 molar potassium chloride.

Once the physiological variables were stable, effects of each drug on hemodynamics and ECG were determined based on two different protocols: 1) infusion of a compound of the present invention to determine the effects of the compound itself mainly on contractility, and other hemodynamic indices; and 2) infusion of a compound of the present invention while the system was challenged with 0.5 ug/kg isoproterenol to determine the beta-adrenergic antagonism properties of each tested compound.

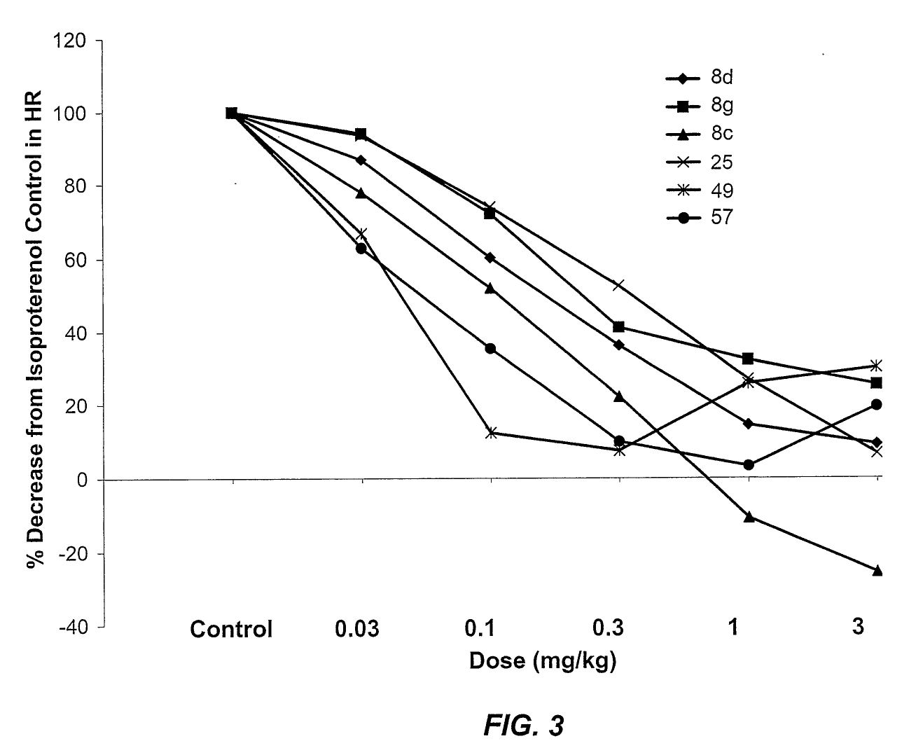

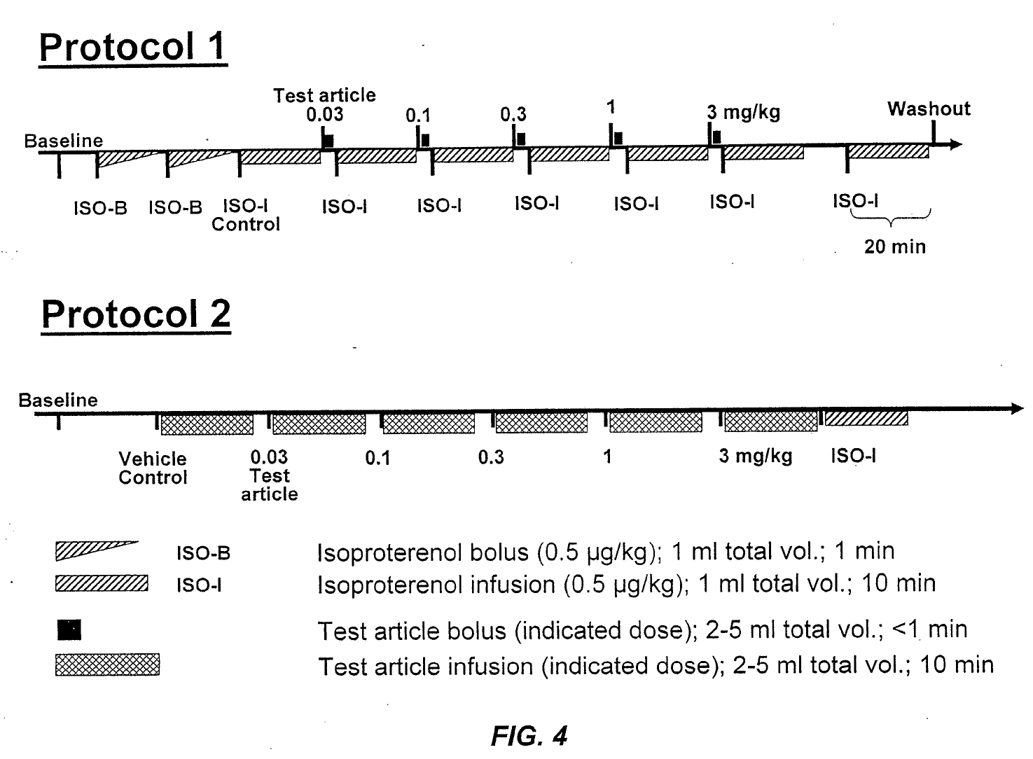

As shown in FIG. 2, the majority of compounds tested were associated with a dose-dependent increase in left ventricular contractibility. In addition, all of the compounds tested displayed strong beta-adrenergic antagonism under isoproterenol challenge (FIG. 3).

### Example 52

### In vitro PDE3 and β-Andrenergic Receptor Binding and Inhibition Studies on PDE3 Inhibition and B-Adrenergic Blockade

The ability of Compounds 25 and 8c to inhibit PDE3 and β-adrenergic receptor activity was examined both in vitro and in vivo. The results were compared to those obtained using carvedilol and/or atenolol. Non-selective β-adrenergic and β₁-adrenergic radioligand binding assays were performed using rat cortical membranes; β₂-adrenergic receptor assays were performed using membranes isolated from CHO cells expressing human, recombinant b₂-adrenergic receptor; non-selective α1-adrenergic receptor assays were performed using rat forebrain membranes; and PDE3 binding assays were performed on human platelets. The resulting affinity data for compounds 25 and 8c are set forth in Table 4. Values in percent illustrate percent inhibition at 1000 nM concentration.

TABLE 1
In Vitro β-adrenergic and PDE3 Inhibition Studies

| | | Non- | | | α1- |
|---|---|---|---|---|---|
| | PDE3 | selective β- | β1- | β2- | adrenergic |
| | IC50, | adrenergic | adrenergic | adrenergic | IC50, |
| | nM | IC50, nM | IC50, nM | IC50, nM | nM |
| Compound | 19 | 3 | 2 | 1 | 349 |
| 25 | | | | | |
| Compound | 150 | 14 | 5 | 0.8 | 226 |
| 8c | | | | | |
| Carvedilol | — | 2 | 1 | <1 | 4 |
| Atenolol | — | 1820 | 23% | 16% | inactive |

### Example 53

### In vitro Studies on PDE3 Inhibition and B-Adrendergic Blockade

In vitro studies were performed using rabbit ventricular tissue, to examine the effects of Compounds 25 and 8c on myocyte contractility. New Zealand male rabbits (1.5-2.5 kg) were anesthetized with ketamine (50 mg/kg) and xylazine (6 mg/kg)-IM. The heart was exposed and removed rapidly to ensure that the aorta remained intact. The ventricular tissue was digested enzymatically. Sarcomere shortening was measured in the presence of 2 mM calcium at 37° C. using an IonOptix system; reported values were at 2 Hz pacing frequency.

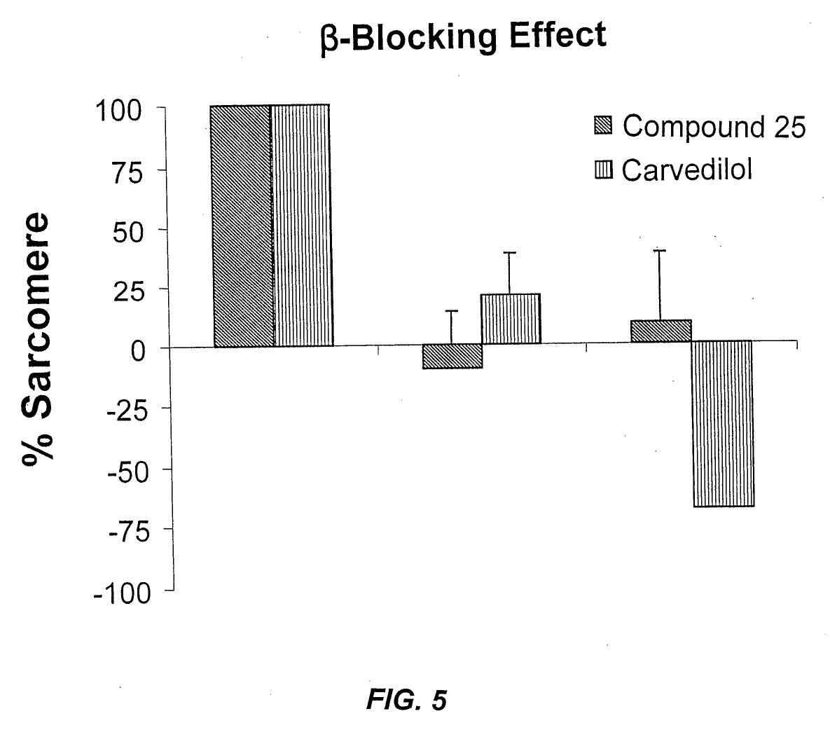

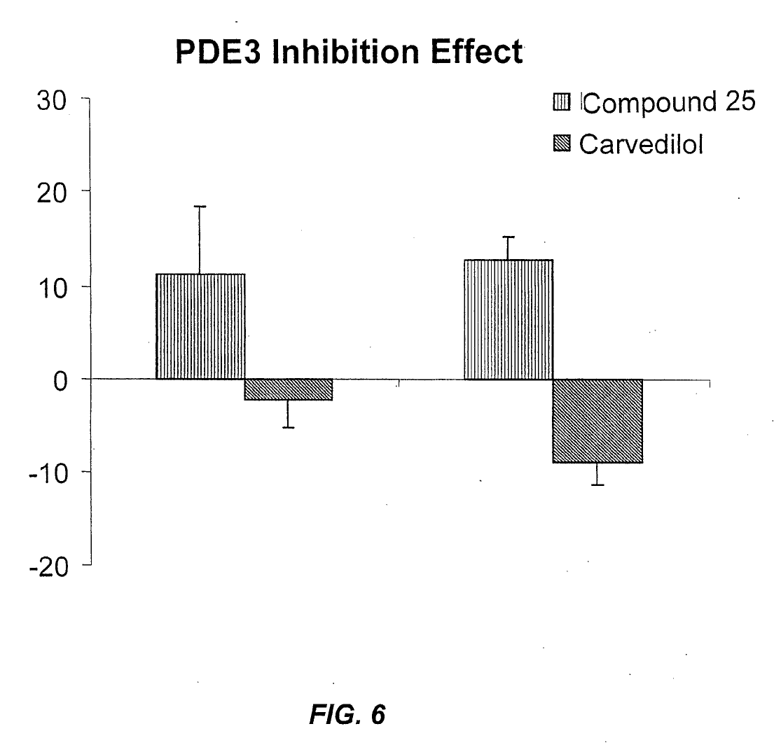

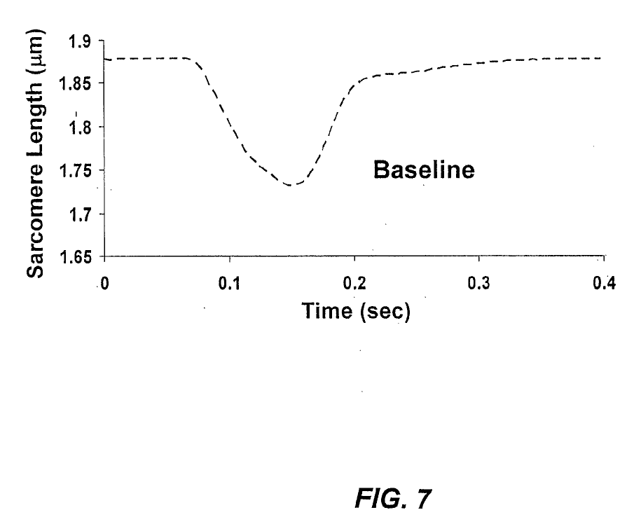

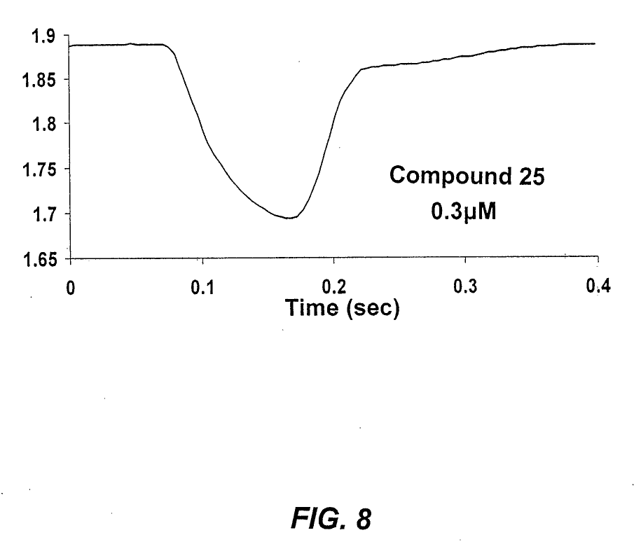

Unlike carvedilol, Compound 25 increased contractility (sarcomere shortening) in isolated ventricular myocytes, while simultaneously blocking the effects of 0.1 μM isoproterenol. The effects of Compound 25 and carvedilol on β-blocking and PDE3 inhibition were compared. FIG. 5 demonstrates the comparative effect of Compound 25 and carvedilol versus the isoproterenol effect. FIG. 6 demonstrates the comparative effect of Compound 25 and carvedilol versus baseline conditions. Representative sarcomere length raw data (carvedilol at 0.1 μM) appear in FIGS. 7 and 8. All values throughout are mean ±SEM.

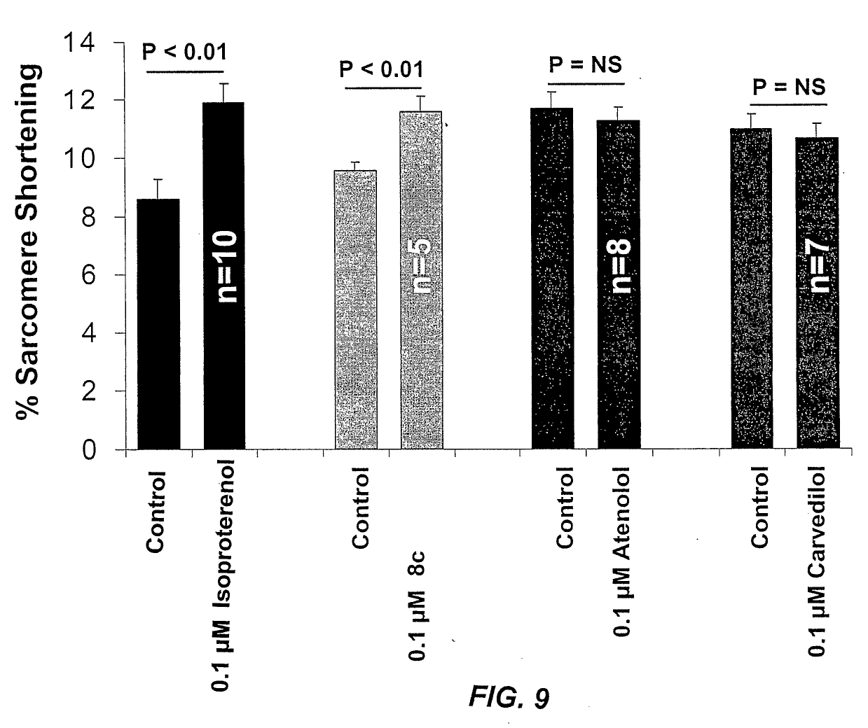

Similarly, unlike carvedilol and atenolol, Compound 8c increased contractility in isolated ventricular myocytes, while simultaneously blocking the effects of 0.1 mM isoproterenol (not shown). Sarcomere shortening induced by Compound 8c, as compared to atenolol or carvedilol is shown in FIG. 9.

### Example 54

### In vivo Studies on PDE3 Inhibition and B-Adrenergic Blockade

White New Zealand male rabbits (2-3 kg) were anesthetized and ventilated (95% O₂+5% CO₂) with 1.25% isoflurane. Each animal was instrumented for LV pressure (Millar, through left carotid artery), arterial pressure (left femoral artery), infusion (jugular vein), and electrocardiogram. The animals were administered intravenously based on one of these protocols:

Protocol 1: β-adrenergic blockade (isoproterenol challenge). See FIG. 1.

Protocol 2: PDE3 inhibition (effects of compounds when administered alone). See FIG. 2.

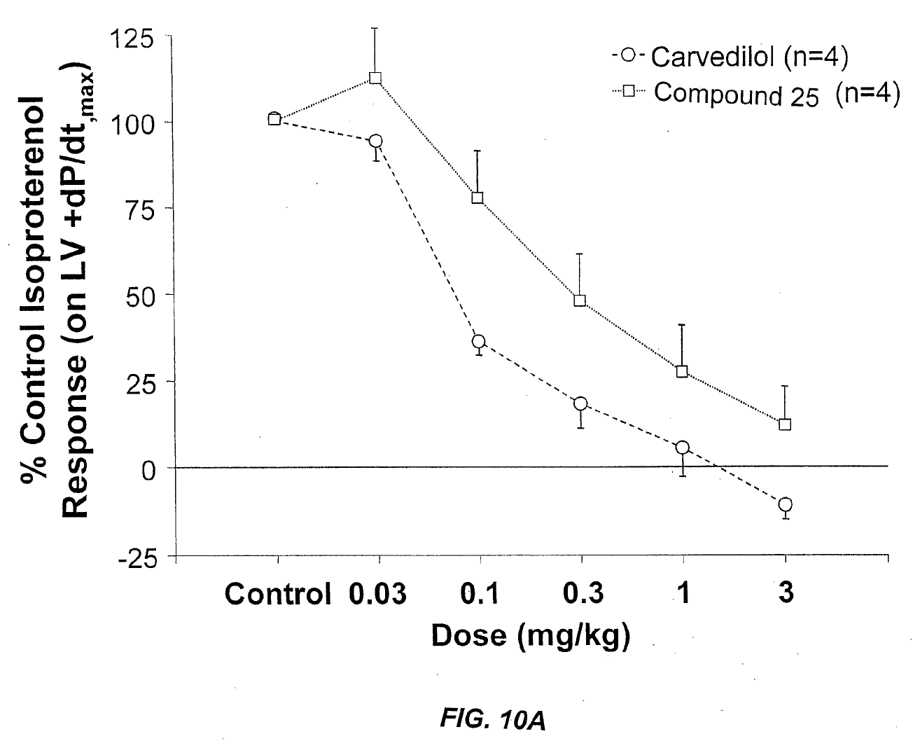

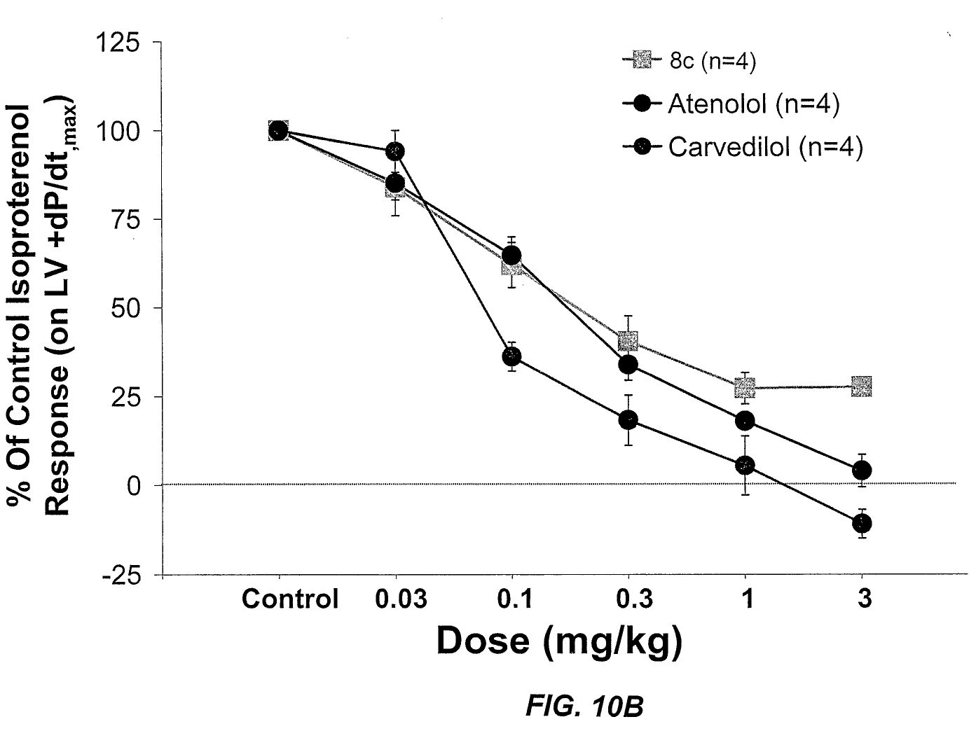

Similar to carvedilol, Compound 25 exhibited a potent dose-dependent β-adrenergic antagonism on left ventricular contractility during isoproterenol challenge (0.5 mg/kg). See FIG. 10a. Similar to both carvedilol and atenolol, Compound 8c also exhibited a potent dose-dependent β-adrenergic antagonism on left ventricular contractility during isoproterenol challenge (0.5 mg/kg). See FIG. 10b.

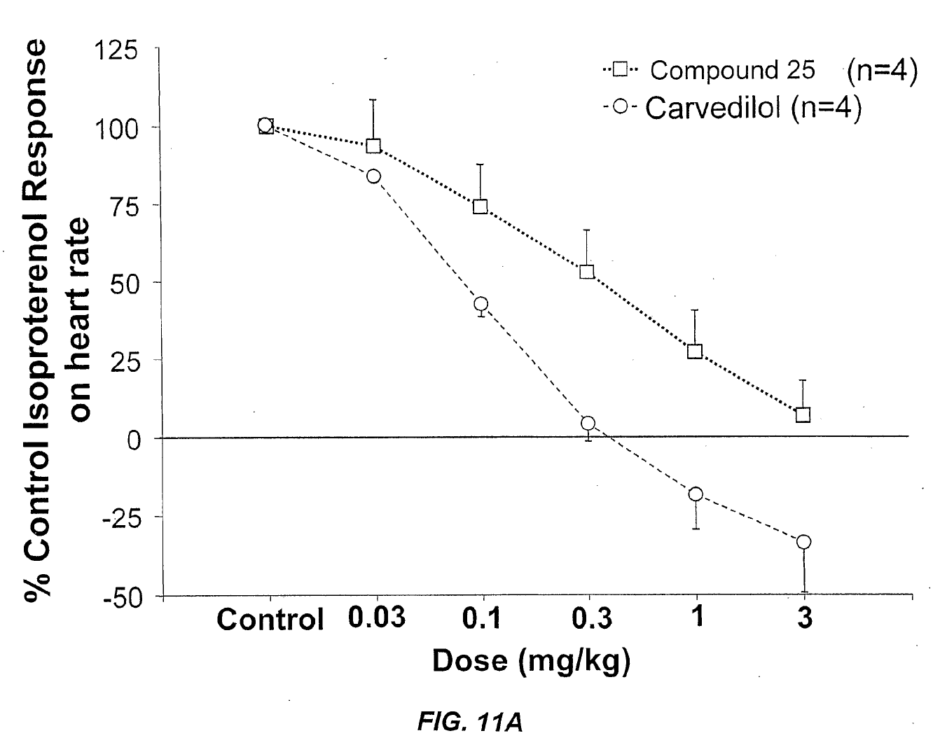

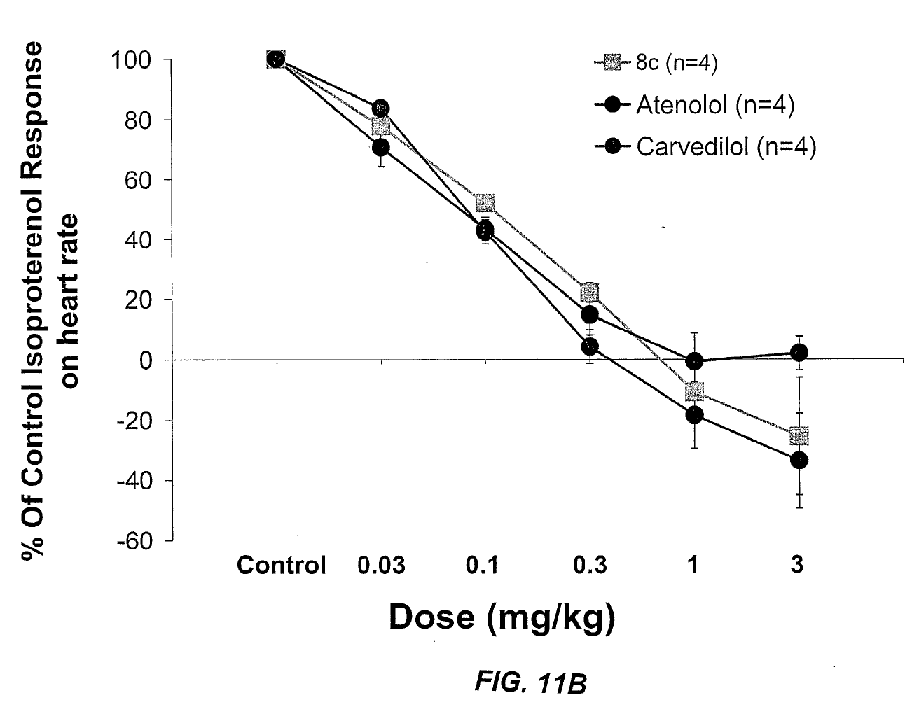

Like carvedilol and atenolol, both Compound 25 and Compound 8c exhibited a dose-dependent β-adrenergic antagonism on heart rate (HR) during isoproterenol challenge (0.5 μg/kg). Carvedilol ED₅₀=0.1 mg/kg; Compound 25 ED₅₀=0.3 mg/kg. See FIGS. 11a and 11b.

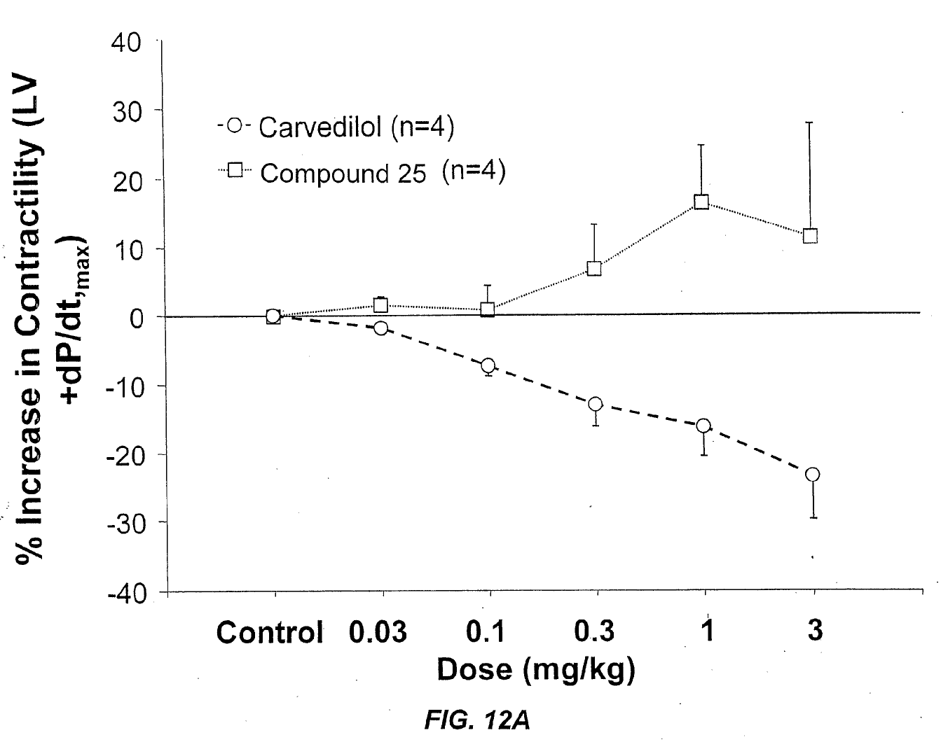

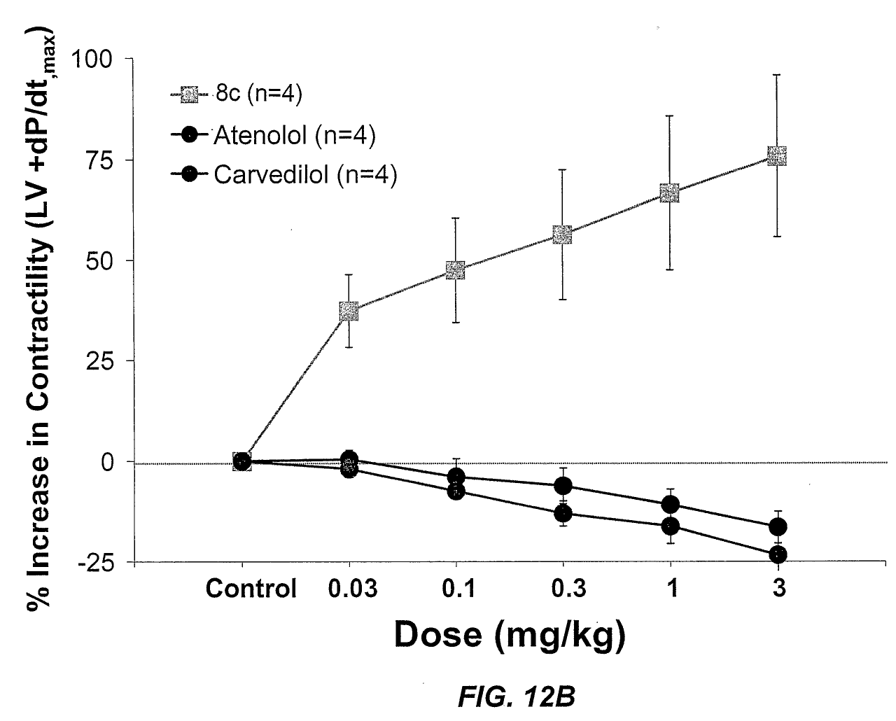

While carvedilol and atenolol decreased contractility when administered alone (protocol 2), Compound 25 and Compound 8c increased or maintained contractility in a dose-dependent manner relative to control. See FIGS. 12a and 12b.

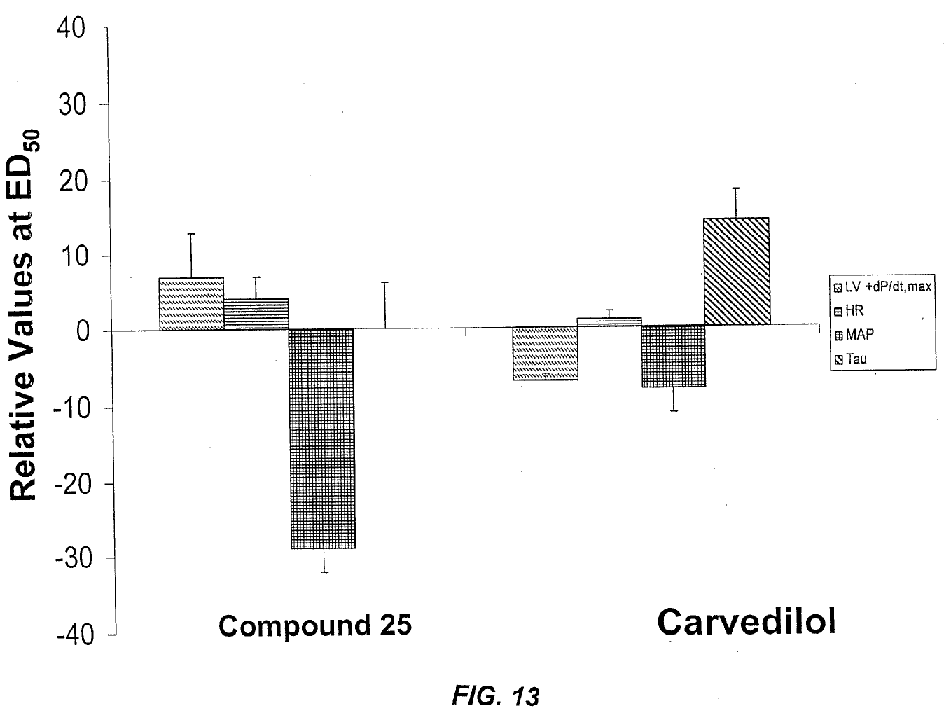

At doses that diminished isoproterenol-dependent increase in HR by 50% (ED₅₀), Compound 25 (0.3 mg/kg) increased contractility (LV+dP/dtmax), while carvedilol (0.1 mg/kg) decreased this parameter. Both drugs had the similar effect on HR. Compound 25 induced a more potent decrease in mean arterial pressure (MAP) than carvedilol. While carvedilol had a negative lusitropic effect, Compound 25 did not change the relaxation properties (Tau) compared to control (Protocol 1). See FIG. 13.

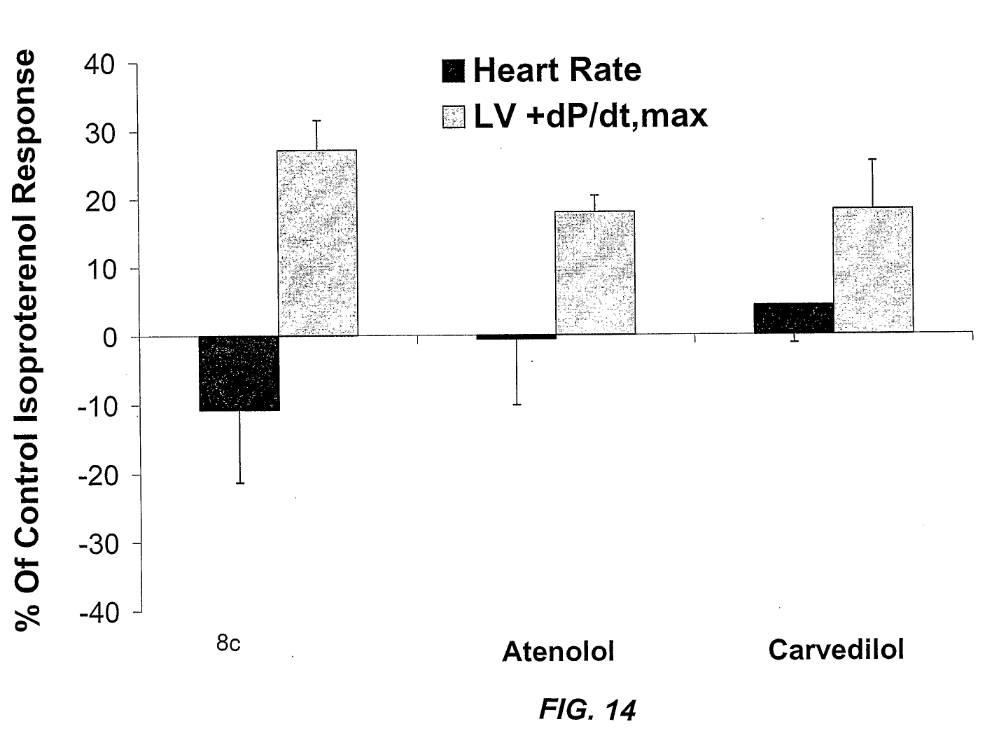

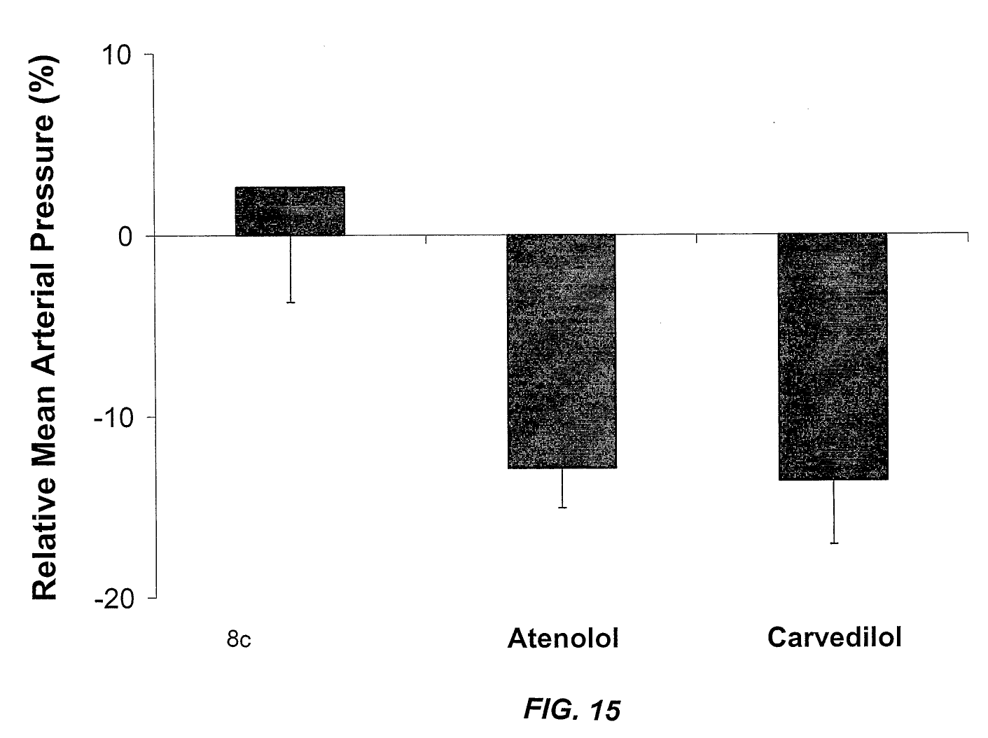

Similarly, at doses that completely diminished isoproterenol-dependent increase in heart rate, left ventricular contractility was decreased by 82±2% (% of isoproterenol effect) with atenolol (1 mg/kg), 82±7% with carvedilol (0.3 mg/kg), while only by 73±4% with Compound 8c (1 mg/kg) due to its PDE3 inhibition activity (Protocol 1) (mean±SEM). See FIG. 14. In addition, at doses that completely diminished isoproterenol-dependent increase in heart rate, carvedilol and atenolol decreased mean arterial pressure, while Compound 8c did not have a significant effect (Protocol 2)(mean±SEM), as shown in FIG. 15.

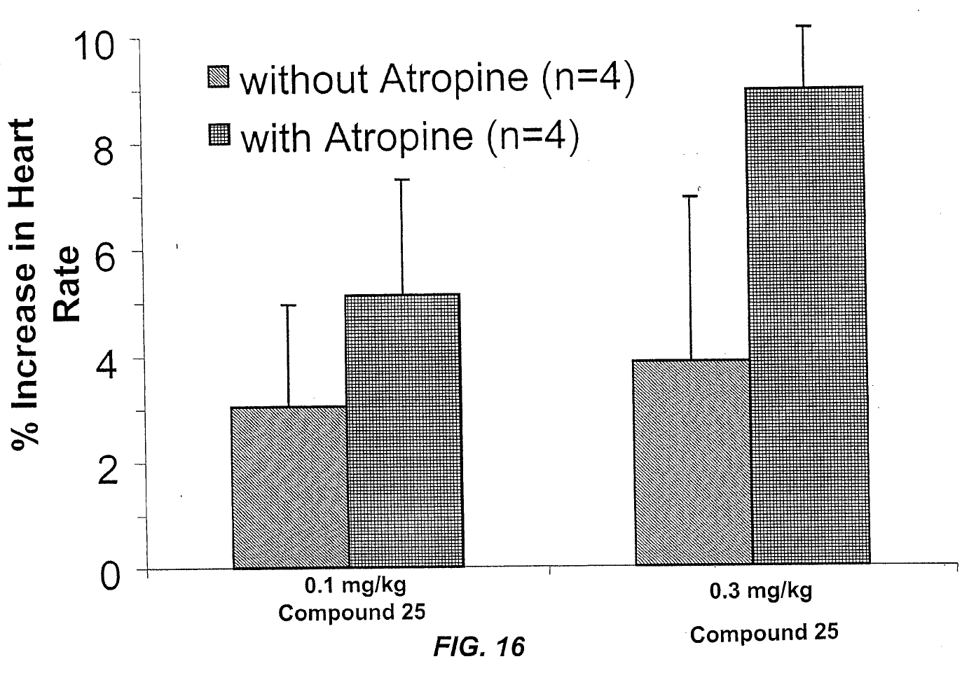

Compound 25 had minimal effect on heart rate and these were similar with and without atropine administration and bilateral vagotomy (Protocol 2). See FIG. 16.

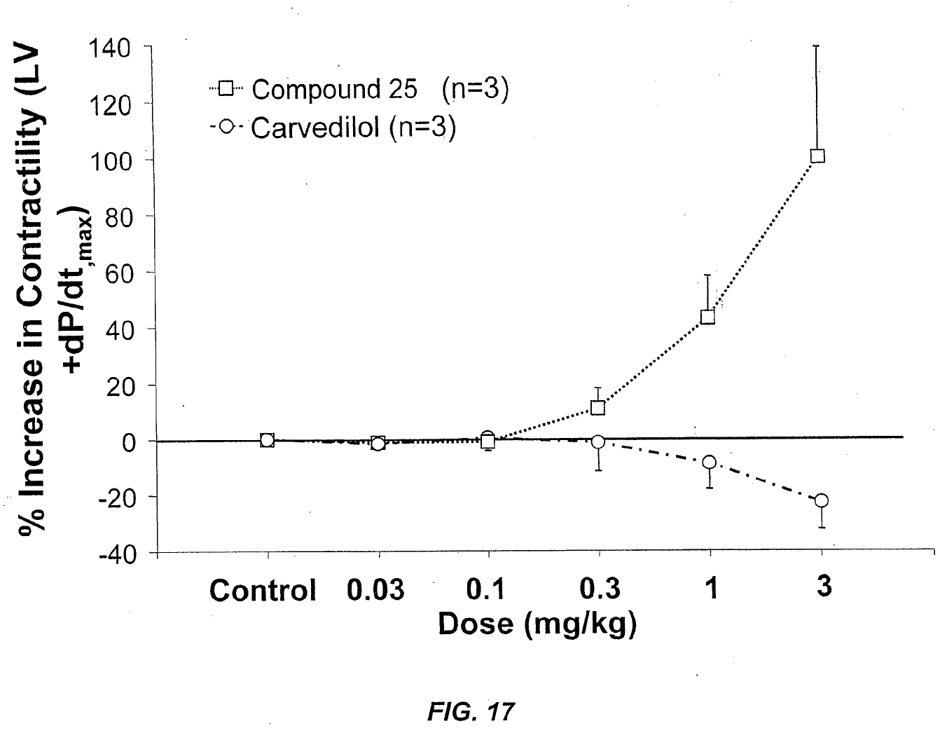

Using experimental protocols and methods similar to that of Example 57, the effects of Compound 25 and carvedilol on contractility was determined in naive anesthetized canines. Compound 25 increased contractility in a dose-dependent manner, while carvedilol decreased this parameter at the higher doses when administered alone (protocol 2). See FIG. 17.

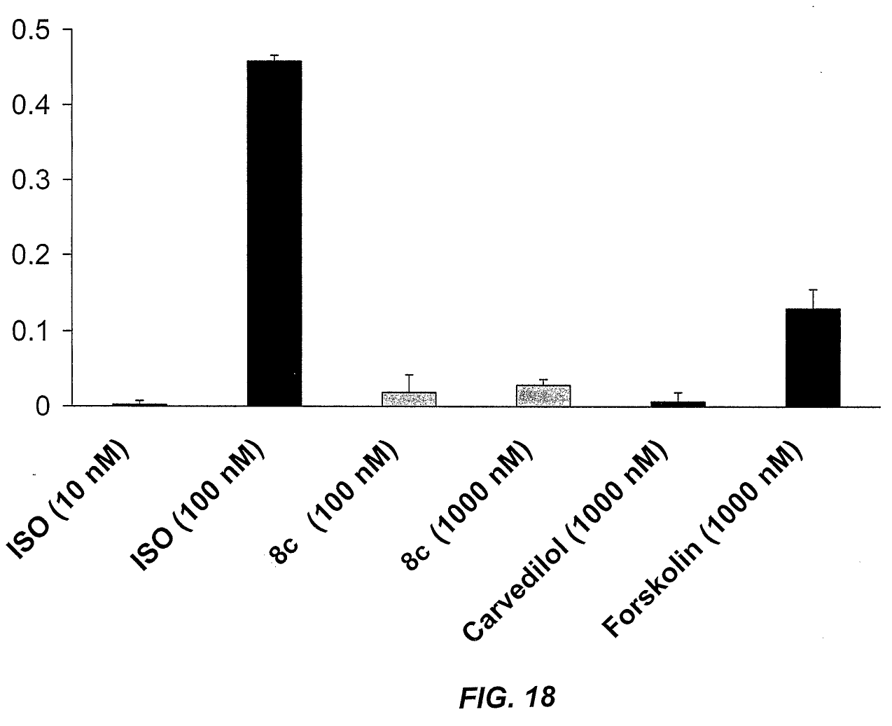

The effect of Compound 8c on cyclic-AMP production was determined in human fetal cardiac fibroblasts by fluorescence, presented in FIG. 18 as [F−Fₒ)/Fₒ], mean±SEM, N=4. IBMX (750 μM) was present in all assays. Compound 8c did not possess intrinsic sympathomimetic activity, as compared to isoproterenol or forskolin, at any concentration tested.

These data demonstrate that unlike carvedilol and atenolol, both Compound 8c and Compound 25 increased contractility in isolated ventricular myocytes, while blocking the effects of isoproterenol. Similar to carvedilol and atenolol, Compound 8c and Compound 25 exhibited a potent dose-dependent β-adrenergic antagonism on left ventricular contractility during isoproterenol challenge. While carvedilol and atenolol decreased left ventricular contractility when administered alone, both Compound 25 and Compound 8c increased left ventricular contractility in a dose-dependent manner, but did not increase heart rate. Similar to carvedilol and atenolol, Compound 8c and Compound 25 exhibited a potent dose-dependent β-adrenergic antagonism on heart rate during isoproterenol challenge. Furthermore, at doses that completely diminished isoproterenol-dependent increase in heart rate, Compound 8c decreased left ventricular contractility to a lesser extent than carvedilol and atenolol and did not decrease mean arterial pressure as did carvedilol and atenolol. Similarly, at doses that diminished isoproterenol-dependent increase in heart rate by 50% (ED50), Compound 25 increased left ventricular contractility, while carvedilol decreased this parameter. Both drugs had the same effect on heart rate. Compound 25 induced a more potent decrease in mean arterial pressure than carvedilol. While carvedilol had a negative lusitropoic effect, Compound 25 did not change the relaxation properties compared to control. In addition, neither Compound 8c nor Compound 25 possessed intrinsic sympathomimetic activity.

These data establish that both Compound 8c and Compound 25 exhibit a common unique pharmacological profile demonstrating a potent beta-adrenergic blocking activity, while simultaneously providing contractile support through their type-3 phosphodiesterase inhibition. This novel pattern of hemodynamic action, coupled with a lusitropic and non-arrhythmogenic profile, establish that Compound 8c and Compound 25 are useful in the treatment of chronic heart failure.

All publications, patents and patent applications identified above are herein incorporated by reference.

The invention being thus described, it will be apparent to those skilled in the art that the same may be varied in many ways without departing from the spirit and scope of the invention. Such variations are included within the scope of the invention to be claimed.

1. A compound of formula I 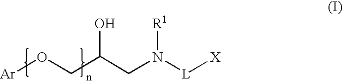
or a pharmaceutically acceptable equivalent thereof, an isomer thereof, a mixture of isomers thereof, a pharmaceutically acceptable salt thereof, a hydrate thereof, a solvate thereof, a metabolite thereof, a prodrug thereof, or an isostere thereof, wherein:
(1) n is 0 or 1;
Ar is an aryl or heteroaryl radical, which aryl or heteroaryl radical is optionally substituted with 1 to 3 substituent(s) selected from R², R³ and R⁴;
(2) R¹ is hydrogen, C₁-C₈ alkyl, C₂-C₈ alkenyl, C₂-C₈ alkynyl, C₃-C₈ cycloalkyl or C₃-C₈ cycloalkenyl;
(3) R², R³ and R⁴ are independently cyano, nitro, halo, trifluoromethyl, trifluoromethoxy, acylaminoalkyl, NHR⁵, —NHSO₂R¹, —NHCONHR¹, C₁-C₄ alkoxy, C₁-C₄ alkylthio, C₁-C₈ alkyl, C₂-C₈ alkenyl or C₂-C₈ alkynyl, wherein one or more —CH₂— group(s) of the alkyl, alkenyl or alkynyl is/are optionally replaced with —O—, —S—, —SO₂— and/or —NR⁵—, and the alkyl, alkenyl or alkynyl is optionally substituted with one or more oxo(s), carbonyl oxygen(s) and/or hydroxyl(s);
(4) L is C₁-C₁₂ alkylene, C₂-C₁₂ alkenylene or C₂-C₁₂ alkynylene, wherein one or more —CH₂— group(s) of the alkylene, alkenylene or alkynylene is/are optionally replaced with —O—, —S—, —SO₂—, —NR⁵—, C₃-C₈ cycloalkylene and/or C₃-C₈ heterocycloalkylene, and the alkylene, alkenylene and alkynylene are unsubstituted or substituted with one or more oxo(s), carbonyl oxygen(s) and/or hydroxyl(s);
(5) R⁵ is hydrogen, a lone pair of electrons, C₁-C₈ alkyl, C₂-C₈ alkenyl or C₃-C₈ alkynyl, which alkyl, alkenyl or alkynyl is optionally substituted with phenyl or substituted phenyl;
(6) X is a moiety of formula A, B, C, D, E, F, G, H, I, J, K, L, M, N, O, P, Q, R, S, T, U, V, W, or Y, 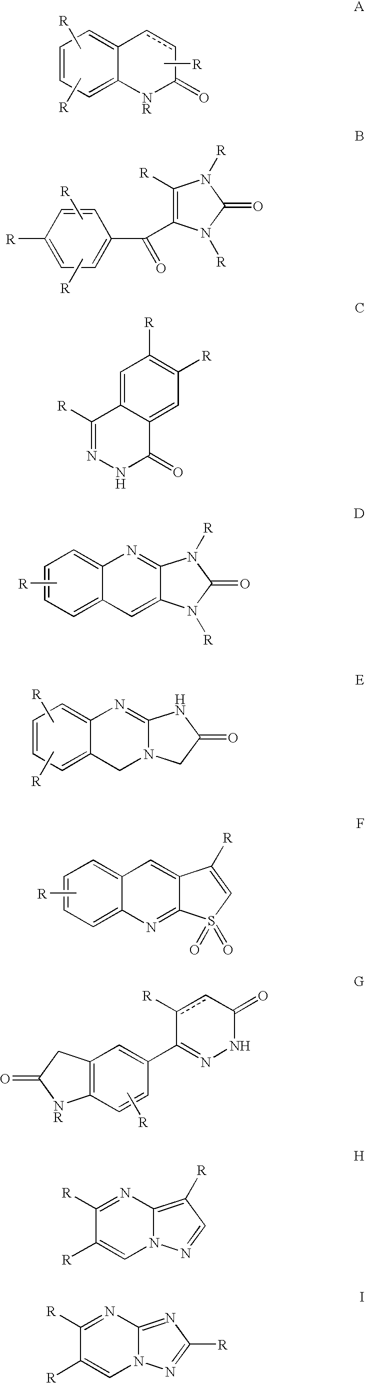 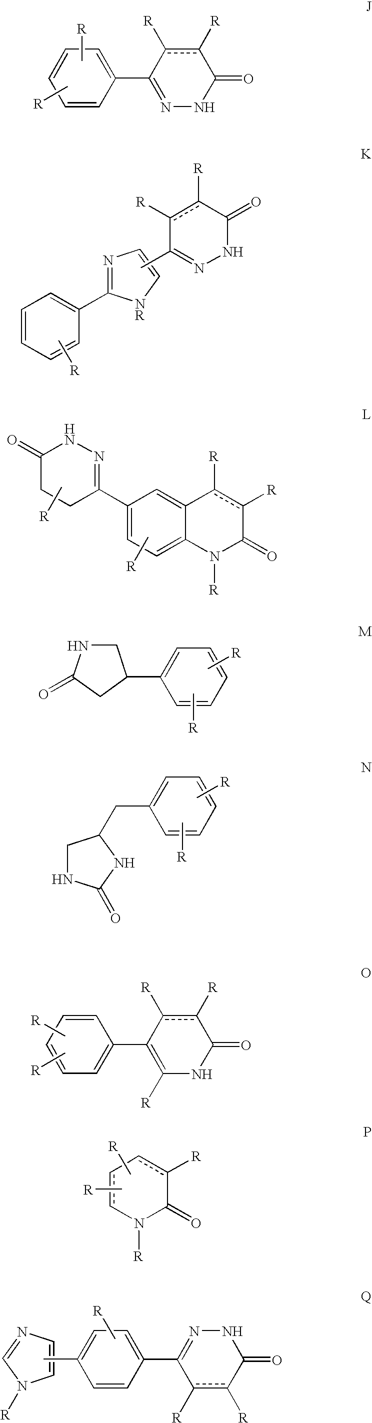 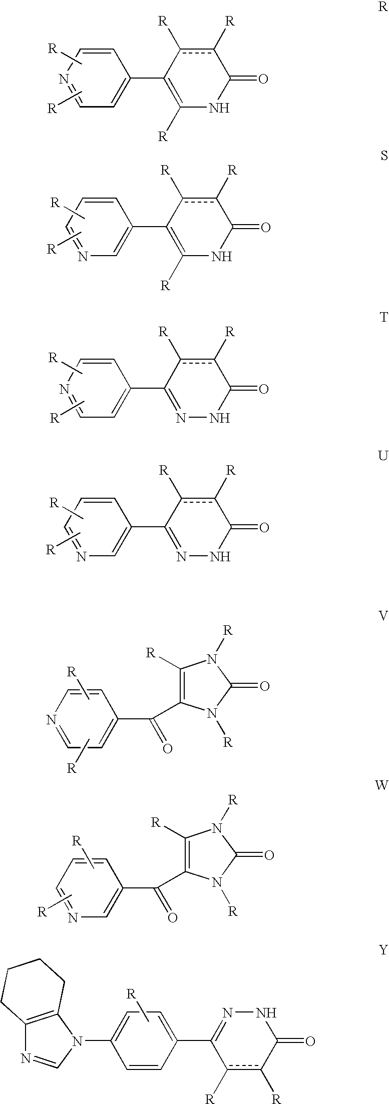
with X bonded to L through any one R; and
each R is independently a direct bond, hydrogen, halo, nitro, cyano, trifluoromethyl, trifluoromethoxy, amino, NR⁵R⁶, C₁-C₄ alkoxy, C₁-C₄ alkylthio, COOR⁷, C₁-C₁₂ alkyl, C₂-C₁₂ alkenyl or C₂-C₁₂ alkynyl, wherein one or more —CH₂— group(s) of the alkyl, alkenyl or alkynyl is/are optionally replaced with —O—, —S—, —SO₂— and/or —NR¹, and the alkyl, alkenyl or alkynyl is optionally substituted with one or more oxo(s), carbonyl oxygen(s) and/or hydroxyl(s).

2. The compound of claim 1, wherein formula I's Ar is phenyl, benzyl, naphthyl or biphenyl.

3. The compound of claim 2, wherein Ar is phenyl which is unsubstituted or substituted with 1 to 3 substituent(s) selected from R², R³ and R⁴, wherein R², R³ and R⁴ are independently cyano, halo, trifluoromethyl, trifluoromethoxy, C₁-C₄ alkoxy, C₁-C₈ alkyl or C₂-C₈ alkenyl, wherein one or more —CH₂— group(s) of the alkyl or alkenyl is/are optionally replaced with —O—, and the alkyl or alkenyl is optionally substituted with oxo.

4. The compound of claim 1, wherein formula (I)'s Ar is chosen from groups Ar₁, Ar₂, Ar₃, Ar₄, Ar₅, Ar₆ and Ar₇: 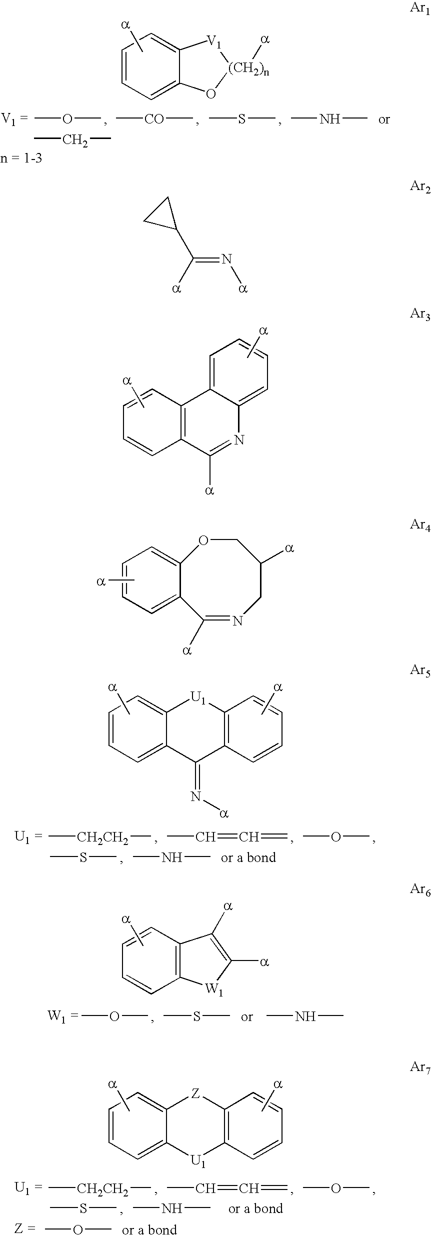
wherein α indicates the position position where Ar may bond.

5. The compound of claim 4, wherein Ar is phenyl or Ar₇, wherein Z is a bond.

6. (canceled)

7. The compound of claim 1, wherein formula I's X is a moiety of formula A, B, C, D, E, F, G, H, I, J, K, L, M, N, O, P, Q, R, S, T, U, V, W, or Y, wherein either (1) Ar is group Ar₇ and Z is a bond, or (2) L is C₁-C₁₂ alkylene, C₂-C₁₂ alkenylene or C₂-C₁₂ alkynylene, and one or more —CH₂— group(s) of the alkylene, alkenylene and alkynylene is/are replaced with C₃-C₈ cycloalkylene and/or C₃-C₈ heterocycloalkylene.

8. The compound of claim 1, wherein formula I's X is a moiety of formula J.

9.-10. (canceled)

11. The compound of claim 1, wherein formula I's L is C₁-C₁₂ alkylene, C₂-C₁₂ alkenylene or C₂-C₁₂ alkynylene, wherein one or more —CH₂— group(s) of the alkylene, alkenylene or alkynylene is/are replaced with C₃-C₈ cycloalkylene and/or C₃-C₈ heterocycloalkylene.

12.-15. (canceled)

16. The compound of claim 1, wherein the R¹ of formula I is hydrogen, C₁-C₈ alkyl, or C₁-C₄ alkyl.

17. (canceled)

18. The compound of claim 1, wherein the n of formula I is 1.

19.-28. (canceled)

29. The compound of claim 1, wherein L is C₁-C₁₂ alkylene, C₂-C₁₂ alkenylene or C₂-C₁₂ alkynylene, wherein one or more —CH₂— group(s) of the alkylene, alkenylene or alkynylene is/are optionally replaced with —O—, —S—, —SO₂— and/or —NR⁵—, and the alkylene, alkenylene and alkynylene are unsubstituted or substituted with one or more oxo(s), carbonyl oxygen(s) and/or hydroxyl(s).

30.-31. (canceled)

32. The compound of claim 1, wherein the compound is a racemic mixture.

33. The compound of claim 1, wherein:
n is 1;
R¹ is hydrogen; and
L is C₁-C₁₂ alkylene, C₂-C₁₂ alkenylene or C₂-C₁₂ alkynylene, wherein one or more —CH₂— group(s) of the alkylene, alkenylene or alkynylene is/are replaced with C₃-C₈ cycloalkylene and/or C₃-C₈ heterocycloalkylene.

34.-41. (canceled)

42. The compound of claim 1, wherein Ar is phenyl which is unsubstituted or substituted with 1 to 3 substituent(s) selected from R², R³ and R⁴.

43.-44. (canceled)

45. The compound of claim 1, wherein the compound is a non-racemic mixture.

46. The compound of claim 1, wherein said compound is selected from the group consisting of:
6-(3-chloro-4-{2-[4-(2-hydroxy-3-phenoxy-propylamino)-piperidin-1-yl]-2-oxo-ethoxy}-phenyl)-4,5-dihydro-2H-pyridazin-3-one (8a),
6-[3-Chloro-4-(2-{4-[3-(2-fluoro-phenoxy)-2-hydroxy-propylamino]-piperidin-1-yl}-2-oxo-ethoxy)-phenyl]-4,5-dihydro-2H-pyridazin-3-one (8b),
6-[3-Chloro-4-(2-{4-[3-(2-chloro-phenoxy)-2-hydroxy-propylamino]-piperidin-1-yl}-2-oxo-ethoxy)-phenyl]-4,5-dihydro-2H-pyridazin-3-one (8c),
6-[4-(2-{4-[3-(2-bromo-phenoxy)-2-hydroxy-propylamino]-piperidin-1-yl}-2-oxo-ethoxy)-3-chloro-phenyl]-4,5-dihydro-2H-pyridazin-3-one (8d),
2-[3-(1-{2-[2-chloro-4-(6-oxo-1,4,5,6-tetrahydro-pyridazin-3-yl)-phenoxy]-acetyl}-piperidin-4-ylamino)-2-hydroxy-propoxy]-benzonitrile (8e),
6-(3-chloro-4-{2-[4-(2-hydroxy-3-o-tolyloxy-propylamino)-piperidin-1-yl]-2-oxo-ethoxy}-phenyl)-4,5-dihydro-2H-pyridazin-3-one (8f),
6-[3-chloro-4-(2-{4-[2-hydroxy-3-(2-trifluoromethyl-phenoxy)-propylamino]-piperidin-1-yl}-2-oxo-ethoxy)-phenyl]-4,5-dihydro-2H-pyridazin-3-one (8g)
6-[3-chloro-4-(2-{4-[2-hydroxy-3-(2-methoxy-phenoxy)-propylamino]-piperidin-1-yl}-2-oxo-ethoxy)-phenyl]-4,5-dihydro-2H-pyridazin-3-one (8h),
6-[3-chloro-4-(2-{4-[2-hydroxy-3-(2-trifluoromethoxy-phenoxy)-propylamino]-piperidin-1-yl}-2-oxo-ethoxy)-phenyl]-4,5-dihydro-2H-pyridazin-3-one (8i),
6-[4-(2-{4-[3-(2-allyl-phenoxy)-2-hydroxy-propylamino]-piperidin-1-yl}-2-oxo-ethoxy)-3-chloro-phenyl]-4,5-dihydro-2H-pyridazin-3-one (8j),
6-[4-(2-{4-[3-(2-acetyl-phenoxy)-2-hydroxy-propylamino]-piperidin-1-yl}-2-oxo-ethoxy)-3-chloro-phenyl]-4,5-dihydro-2H-pyridazin-3-one (8k),
6-[4-(2-{4-[3-(3-bromo-phenoxy)-2-hydroxy-propylamino]-piperidin-1-yl}-2-oxo-ethoxy)-3-chloro-phenyl]-4,5-dihydro-2H-pyridazin-3-one (8l),
3-[3-(1-{2-[2-chloro-4-(6-oxo-1,4,5,6-tetrahydro-pyridazin-3-yl)-phenoxy]-acetyl}-piperidin-4-ylamino)-2-hydroxy-propoxy]-benzonitrile (8m),
6-[4-(2-{4-[3-(4-bromo-phenoxy)-2-hydroxy-propylamino]-piperidin-1-yl}-2-oxo-ethoxy)-3-chloro-phenyl]-4,5-dihydro-2H-pyridazin-3-one (8n),
4-[3-(1-{2-[2-chloro-4-(6-oxo-1,4,5,6-tetrahydro-pyridazin-3-yl)-phenoxy]-acetyl}-piperidin-4-ylamino)-2-hydroxy-propoxy]-benzonitrile (8o),
6-(3-chloro-4-{2-[4-(2-hydroxy-3-p-tolyloxy-propylamino)-piperidin-1-yl]-2-oxo-ethoxy}-phenyl)-4,5-dihydro-2H-pyridazin-3-one (8p),
6-[3-chloro-4-(2-{4-[2-hydroxy-3-(4-methoxy-phenoxy)-propylamino]-piperidin-1-yl}-2-oxo-ethoxy)-phenyl}-4,5-dihydro-2H-pyridazin-3-one (8q),
6-{3-chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (8r),
6-(4-{3-[3-(9H-carbazol-4-yloxy)-2-hydroxy-propylamino]-propoxy}-3-chloro-phenyl)-4,5-dihydro-2H-pyridazin-3-one (29),
6-(4-{2-[3-(9H-carbazol-4-yloxy)-2-hydroxy-propylamino]-ethoxy}-3-chloro-phenyl)-4,5-dihydro-2H-pyridazin-3-one (22),
6-(4-{2-[3-(9H-carbazol-4-yloxy)-2-hydroxy-propylamino]-ethoxy}-3-chloro-phenyl)-4,5-dihydro-2H-pyridazin-3-one (25),
6-[4-(2-{4-[3-(9H-carbazol-4-yloxy)-2-hydroxy-propylamino]-piperidin-1-yl}-2-oxo-ethoxy)-3-chloro-phenyl]-4,5-dihydro-2H-pyridazin-3-one (17, 68),
6-{3-chloro-4-[2-(4-{2-hydroxy-3-[4-(2-methoxy-ethyl)-phenoxy]-propylamino}-piperidin-1-yl)-2-oxo-ethoxy]-phenyl}-4,5-dihydro-2H-pyridazin-3-one (13),
2′-(2-{4-[3-(2-cyano-phenoxy)-2-hydroxy-propylamino]-piperidin-1-yl}-ethoxy)-2-methyl-6-oxo-1,6-dihydro-[3,4′]bipyridinyl-5-carbonitrile (137),
6-[4-(2-{4-[(S)-3-(9H-Carbazol-2-yloxy)-2-hydroxy-propylamino]-piperidin-1-yl}-2-oxo-ethoxy)-3-chloro-phenyl]-4,5-dihydro-2H-pyridazin-3-one (33),
6-(4-{2-[3-(9H-carbazol-2-yloxy)-2-hydroxy-propylamino]-ethoxy}-3-chloro-phenyl)-4,5-dihydro-2H-pyridazin-3-one (34),
6-(4-{3-[(S)-3-(9H-Carbazol-2-yloxy)-2-hydroxy-propylamino]-propoxy}-3-chloro-phenyl)-4,5-dihydro-2H-pyridazin-3-one (35),
6-[3-Chloro-4-(3-{(S)-2-hydroxy-propylamino]-propoxy}-phenyl)-4,5-dihydro-2H-pyridazin-3-one (36),
2-[2-Chloro-4-(6-oxo-1,4,5,6-tetrahydro-pyridazin-3-yl)-phenoxy]-N-(2-{(S)-2-hydroxy-3-[4-(2-methoxy-ethyl)-phenoxy]-propylamino}-2-methyl-propyl)-acetamide (40),
6-[3-Chloro-4-(3-{(S)-2-hydroxy-propylamino]-ethoxy}-phenyl)-4,5-dihydro-2H-pyridazin-3-one (41),
6-{3-Chloro-4-[2-(3-{(S)-2-hydroxy-3-[4-(2-methoxy-ethyl)-phenoxy]-propylamino}-pyrrolidin-1-yl)-2-oxo-ethoxy]-phenyl}-4,5-dihydro-2H-pyridazin-3-one (42),
6-[3-Chloro-4-(3-{(S)-2-hydroxy-propylamino]-butoxy}-phenyl)-4,5-dihydro-2H-pyridazin-3-one (44),
6-[3-Chloro-4-(3-{(S)-2-hydroxy-propylamino]-pentoxy}-phenyl)-4,5-dihydro-2H-pyridazin-3-one (46),
N-{2-[3-(9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (49),
6-{3-Chloro-4-[2-(3-{(S)-2-hydroxy-3-[4-(2-methoxy-ethyl)-phenoxy]-propylamino}-azetidin-1-yl)-2-oxo-ethoxyl]phenyl}-4,5-dihydro-2H-pyridazin-3-one (48),
6-(3-Chloro-4-{2-hydroxy-3-(1H-indol-4-yloxy)-propylamino]-ethoxy}-phenyl)-4,5-dihydro-2H-pyridazin-3-one (52),
6-(3-Chloro-4-{2-hydroxy-3-(3-propylamino-phenoxy)-propylamino]-ethoxy}-phenyl)-4,5-dihydro-2H-pyridazin-3-one (55),
6-(4-{3-[3-(9H-Carbazol-4-yloxy)-2-hydroxy-butylamino]-propoxy}-3-chloro-phenyl)-4,5-dihydro-2H-pyridazin-3-one (56),
6-(4-{3-[3-(9H-Carbazol-4-yloxy)-2-hydroxy-pentylamino]-propoxy}-3-chloro-phenyl)-4,5-dihydro-2H-pyridazin-3-one (57),
6-(4-{3-[3-(9H-Carbazol-4-yloxy)-2-hydroxy-pentylamino]-propoxy}-3-chloro-phenyl)-4,5-dihydro-2H-pyridazin-3-one (61),
6-[6-(2-{4-[3-(9H-Carbazol-4-yloxy)-2-hydroxy-propylamino]-piperidin-1-yl]-4,5-dihydro-2H-pyridazin-3-one (62),
6-[4-(2-{4-[3-(9H-Carbazol-4-yloxy)-2-hydroxy-propylamino]-piperidin-1-yl}-2-oxo-ethoxy)-3-chloro-phenyl]-4,5-dihydro-2H-pyridazin-3-one (63),
6-(4-{3-[3-(9H-Carbazol-4-yloxy)-2-hydroxy-propylamino]-propoxy}-3-chloro-phenyl)-4,5-dihydro-2H-pyridazin-3-one (64),
6-(4-{3-[3-(9H-Carbazol-4-yloxy)-2-hydroxy-propylamino]-propoxy}-3-chloro-phenyl)-4,5-dihydro-2H-pyridazin-3-one (69),
6-(4-{3-[3-(9H-Carbazol-4-yloxy)-2-hydroxy-propylamino]-propoxy}-3-chloro-phenyl)-4,5-dihydro-2H-pyridazin-3-one (70),
N-{2-[3-(3-Bromo-9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (72a),
N-{2-[3-(1-Bromo-9H-carbazol-4-yloxy)-2-hydroxy-propylamino]-2-methyl-propyl}-2-[2-chloro-4-(6-oxo-1,4,5,6-tetrahydro-pyridazin-3-yl)-phenoxy]-acetamide (72b),
2-(2-Chloro-4-(6-oxo-1,4,5,6-tetrahydro-pyridazin-3-yl)-phenoxy]-N-{2-[2-hydroxy-3-(9-methyl-9H-carbazol-yloxy)-propylamino]-2-methyl-propyl}-2-methyl-propyl}-acetamide (74),
6-[4-(2-{3-[3-(9H-Carbazol-4-yloxy)-2-hydroxy-propylamino]-azetidin-1-yl}-2-oxo-ethoxy)-3-chloro-phenyl]-4,5-dihydro-2H-pyridazin-3-one (75),
9-(2-methoxy-ethyl)-4-oxiranylmethoxy-9H-carbazole (80), 2-[2-chloro-4-(6-oxo-1,4,5,6-tetrahydro-pyridazin-3-yl)-phenoxy]-N-(2-{2-hydroxy-3-[9-(2-methoxy-ethyl)-9H-carbazol-4-yloxy]-propylamino}-2-methyl-propyl)-acetamide (81),
Benzoic acid 2-(4-oxiranylmethoxy-carbazol-9-yl)-ethyl ester (84), 2-[2-Chloro-4-(6-oxo-1,4,5,6-tetrahydro-pyridazin-3-yl)-phenoxy]-N-(2-{2-hydroxy-3-[9-(2-hydroxy-ethyl)-9H-carbazol-4-yloxy]-propylamino}-2-methyl-propyl)-acetamide (85),
5-[4-(3-amino-propoxy)-phenyl]-6-methyl-2-oxo-1,2-dihydro-pyridine-3-carbonitrile (90a),
5-(4-{3-[3-(9H-carbazol-4-yloxy)-2-hydroxy-propylamino]-propoxy}-phenyl)-6-methyl-2-oxo-1,2-dihydro-pyridine-3-carbonitrile (91),
5-[4-(4-amino-butoxy)-3-chloro-phenyl]-6-methyl-2-oxo-1,2-dihydro-pyridine-3-carbonitrile (90b),
5-[4-(5-amino-pentyloxy)-3-chloro-phenyl]-6-methyl-2-oxo-1,2-dihydro-pyridine-3-carbonitrile (90c),
6-[4-(3-{(S)-2-hydroxy-propylamino]-butoxy}-phenyl)-4,5-dihydro-2H-pyridazin-3-one (92),
5-(4-{5-[3-(9H-carbazol-4-yloxy)-2-hydroxy-propylamino]-pentyloxy}-3-chloro-phenyl)-6-methyl-2-oxo-1,2-dihydro-pyridine-3-carbonitrile (93),
[4-(5-cyano-2-methyl-6-oxo-1,6-dihydro-pyridin-3-yl)-phenoxy]-acetic acid (97),
5-[4-(2-{4-[3-(9H-carbazol-4-yloxy)-2-hydroxy-propylamino]-piperidin-1-yl}-2-oxo-ethoxy)-phenyl]-6-methyl-2-oxo-1,2-dihydro-pyridine-3-carbonitrile (98),
N-{2-[3-(9H-carbazol-4-yloxy)-2-hydroxy-propylamino]-2-methyl-propyl}-2-[2-chloro-4-(5-cyano-2-methyl-6-oxo-1,6-dihydro-pyridin-3-yl)-phenoxy]-acetamide (99), and
6-[4-(2-{3-[3-(9H-Carbazol-4-yloxy)-2-hydroxy-propylamino]-pyrrolidin-1-yl}-2-oxo-ethoxy)-3-chloro-phenyl]-4,5-dihydro-2H-pyridazin-3-one (139).

47.-108. (canceled)

109. A pharmaceutical composition comprising the compound of claim 1 and a pharmaceutically acceptable carrier.

110.-111. (canceled)

112. A method of inhibiting β-adrenergic receptors and/or inhibiting phosphodiesterase, comprising administering an effective amount of the pharmaceutical composition of claim 109 to an animal in need of such treatment.

113. A method for regulating calcium homeostasis, comprising administering an effective amount of the pharmaceutical composition of claim 109 to an animal in need of such regulation.

114. A method for treating a disease, disorder or condition in which disregulation of calcium homeostasis is implicated, comprising administering an effective amount of the pharmaceutical composition of claim 109 to an animal in need of such treatment.

115.-118. (canceled)
